(19) United States
(12) Patent Application Publication
(10) Pub. No.: US 2025/0303068 A1
(43) Pub. Date: Oct. 2, 2025

(54) MODULAR MEDICAMENT INJECTOR AND APPLICATOR AND INTERCHANGEABLE POD THEREFOR

(71) Applicant: DIFINITY SOLUTIONS INC., Vancouver (CA)

(72) Inventors: David Sanghyuck Kim, Vancouver (CA); Damien Tak, Vancouver (CA)

(73) Assignee: DIFINITY SOLUTIONS INC., Vancouver (CA)

(21) Appl. No.: 18/619,465

(22) Filed: Mar. 28, 2024

Related U.S. Application Data
(63) Continuation of application No. PCT/CA2024/050390, filed on Mar. 28, 2024.

(30) Foreign Application Priority Data
Mar. 28, 2024 (WO) PCT/CA2024/050390

(51) Int. Cl. A61M 5/28; A61M 5/20; A61M 5/32

(52) U.S. Cl. CPC A61M 5/28; A61M 5/2033; A61M 5/322

(57) ABSTRACT

There is provided according to one aspect, a medicament injector including an applicator with a drive element and an interchangeable pod or cartridge assembly loadable into the applicator so as to couple thereto. Retraction of the pod or cartridge assembly actuates the drive element to engage the pod or cartridge assembly so as to output a medicament therefrom. The applicator is configured to automatically eject the pod or cartridge assembly from the applicator thereafter. The medicament injector includes a barrier member configured such that removal thereof to enable access to a medicament requires the pod or cartridge assembly to couple to and/or be inserted within the applicator according to another aspect. The barrier member is snap-fit in place until the pod or cartridge assembly is inserted into the applicator according to an additional aspect.

### BACKGROUND OF THE INVENTION

### Field of the Invention

There is provided a modular medicament injector. In particular, there is provided a modular medicament injector, an applicator therefor, an interchangeable pod or cartridge assembly therefor and a pod cap assembly thereof.

### Description of the Related Art

United States Patent Application No. 20230019820 to Kim et al. discloses a medicament injector. The medicament injector includes an applicator with a cartridge-accepting receptacle. The medicament injector includes interchangeable, pre-filled medicament-containing cartridges. Each cartridge includes a barrier member having a closed position in which access to the medicament is inhibited. The barrier member is movable from the closed position to an open position, in which the medicament is accessible via the applicator for administration, when the cartridge is inserted into the receptacle. The barrier member moves back to the closed position when the cartridge is automatically removed from the receptacle once the medication is delivered. This allows for plurality of pre-filled medicament cartridges to be selected on demand via a need-based scenario for quick assembly with the medicament injector for use in various clinical scenarios to reduce the bulk, errors, complexity, and steps in medicament delivery while increasing the versatility, safety, and speed of medicament delivery in time critical situations.

Additional non-limiting examples of injectors and syringes are described in: United States Patent Application Publication No. 2005/0283120 A1 to Wang; U.S. Pat. No. 7,717,877 to Lavi et al.; International Patent Application Publication No. WO 2013/065055 A1 to Lev; U.S. Pat. No. 9,028,453 to Jennings; U.S. Pat. Nos. 9,440,026; 10,610,646 to Schmid et al.; U.S. Pat. No. 10,646,654 to Mosebach et al.; and U.S. Pat. No. 11,160,929 to Dahmani.

The above-described prior art may suffer a number of disadvantages.

### BRIEF SUMMARY OF INVENTION

There is provided, and it is an object to provide, an improved modular medicament injector, and an applicator, interchangeable pod or cartridge assembly and pod cap assembly therefor, disclosed herein.

There is accordingly provided a medicament injector according to one aspect. The medicament injector includes an applicator with a drive element. The medicament injector includes an interchangeable pod or cartridge assembly front-loadable into the applicator so as to couple thereto, with retraction thereof actuating the drive element to engage the pod or cartridge assembly so as to output a medicament therefrom. The applicator is configured to automatically eject the pod or cartridge assembly thereafter.

There is further provided a medicament injector according to yet another aspect. The medicament injector includes an applicator. The medicament injector includes an interchangeable pod or cartridge assembly. The pod or cartridge assembly includes a cartridge (and/or syringe and needle assembly) shaped to receive or contain a medicament therewithin. Alternatively, the pod or cartridge assembly may not include but may be shaped to receive the cartridge (and/or syringe and needle assembly). The pod or cartridge assembly is resiliently biased to extend about a needle in a locked protected needle pre-injection mode, with retraction thereof into the applicator functioning to deploy the needle. The medicament is biased outwards from the needle when the pod or cartridge assembly is inserted into the applicator and the pod or cartridge assembly is moved from the locked protected needle pre-injection mode to an unlocked protected needle pre-injection mode and then to a partially retracted mode or injection mode. The pod or cartridge assembly is configured to be automatically ejected from the applicator thereafter following administration of the medicament.

There is also provided a medicament injector according to a further aspect. The medicament injector includes an applicator. The medicament injector includes an interchangeable pod or cartridge assembly with a cartridge (and/or syringe and needle assembly) shaped to receive or contain a medicament therewithin. Alternatively, the pod or cartridge assembly may not include but may be shaped to receive the cartridge (and/or syringe and needle assembly). The medicament injector includes a barrier member coupled to the pod or cartridge assembly. The barrier member is configured such that removal thereof to enable access to the medicament requires the pod or cartridge assembly to be coupled to the applicator.

There is also provided a medicament injector according to another aspect. The medicament injector includes an applicator. The medicament injector includes an interchangeable pod or cartridge assembly including a cartridge (and/or syringe and needle assembly) shaped to receive or contain a medicament therewithin. Alternatively, the pod or cartridge assembly may not include but may be shaped to receive the cartridge (and/or syringe and needle assembly). The pod or cartridge assembly includes a barrier member coupled to the pod or cartridge assembly. The pod or cartridge assembly includes a locking mechanism which inhibits removal of the barrier member until the pod or cartridge assembly is inserted within the applicator.

There is further provided a medicament injector according to an additional aspect. The medicament injector includes an applicator. The medicament injector includes an interchangeable pod or cartridge assembly with a cartridge (and/or syringe and needle assembly) shaped to receive or contain a medicament therewithin. Alternatively, the pod or cartridge assembly may not include but may be shaped to receive the cartridge (and/or syringe and needle assembly). The medicament injector includes a pod cap assembly coupled to and configured to be removable from the pod or cartridge assembly upon the pod or cartridge assembly being inserted within the applicator.

There is also provided a medicament injector according to yet another aspect. The medicament injector includes an applicator. The medicament injector includes an interchangeable pod or cartridge assembly including a cartridge (and/or syringe and needle assembly) shaped to receive or contain a medicament therewithin. Alternatively, the pod or cartridge assembly may not include but may be shaped to receive the cartridge (and/or syringe and needle assembly). The pod or cartridge assembly has a needle end. The medicament injector includes a barrier member configured to enclose the needle end of the pod or cartridge assembly until the pod or cartridge assembly is inserted within the applicator.

There is yet further provided a medicament injector according to an additional aspect. The medicament injector includes an applicator. The medicament injector includes an interchangeable pod or cartridge assembly with a cartridge (and/or syringe and needle assembly) shaped to receive or contain a medicament therewithin. Alternatively, the pod or cartridge assembly may not include but may be shaped to receive the cartridge (and/or syringe and needle assembly). The pod or cartridge assembly has a needle end. The medicament injector includes a barrier member configured to inhibit access to the pod or cartridge assembly via the needle end until the pod or cartridge assembly is inserted within the applicator.

There is additionally provided a medicament injector according to another aspect. The medicament injector includes an applicator. The medicament injector includes an interchangeable pod or cartridge assembly with a cartridge (and/or syringe and needle assembly) shaped to receive or contain a medicament therewithin. Alternatively, the pod or cartridge assembly may not include but may be shaped to receive the cartridge (and/or syringe and needle assembly). The pod or cartridge assembly includes a reversible barrier member coupled to the pod or cartridge assembly thereof. The reversible barrier member is configured to inhibit access to a needle until the pod or cartridge assembly is inserted within the applicator.

There is further provided a medicament injector according to an additional aspect. The medicament injector includes an applicator. The medicament injector includes an interchangeable pod or cartridge assembly with and/or operatively connected to a needle. The pod or cartridge assembly includes a reversible barrier member operatively connected thereto. The reversible barrier member is configured to inhibit access to the needle until the pod or cartridge assembly is inserted within the applicator.

There is also provided a medicament injector according to another aspect. The medicament injector includes an applicator. The medicament injector includes an interchangeable pod or cartridge assembly with a cartridge (and/or syringe and needle assembly) shaped to receive or contain a medicament therewithin. Alternatively, the pod or cartridge assembly may not include but may be shaped to receive the cartridge (and/or syringe and needle assembly). The medicament injector includes a barrier member coupled to the pod or cartridge assembly. The barrier member is configured such that removal thereof to enable access to the medicament requires the pod or cartridge assembly to be inserted within and couple to the applicator. The barrier member is configured to render the medicament inaccessible when the pod or cartridge assembly is not inserted into the applicator.

There is also provided a medicament injector according to a further aspect. The medicament injector includes an applicator. The medicament injector includes an interchangeable pod or cartridge assembly with a cartridge (and/or syringe and needle assembly) shaped to receive or contain a medicament therewithin. Alternatively, the pod or cartridge assembly may not include but may be shaped to receive the cartridge (and/or syringe and needle assembly). The medicament injector includes a safeguard mechanism coupled to the pod or cartridge assembly. The safeguard mechanism is configured to enable axial movement of the pod or cartridge assembly relative to the applicator during an injection mode. The safeguard mechanism being configured, upon the pod or cartridge assembly being removed from the applicator thereafter, to inhibit reinsertion of the pod or cartridge assembly into the applicator once more.

There is further provided a medicament injector according to another aspect. The medicament injector includes an applicator. The medicament injector includes an interchangeable pod or cartridge assembly shaped to be insertable within the applicator. The pod or cartridge assembly is configured to receive or include a cartridge (and/or syringe and needle assembly) thereof shaped to receive or contain a medicament therewithin. Alternatively, the pod or cartridge assembly may not include but may be shaped to receive the cartridge (and/or syringe and needle assembly). The medicament injector includes a safeguard mechanism. The safeguard mechanism includes a sleeve which extends about the cartridge (and/or syringe and needle assembly) thereof. The safeguard mechanism includes one or more latch members which bias and/or compress the sleeve to hold the cartridge (and/or syringe and needle assembly) thereof in place in a pre-injection mode. The one or more latch members are configured to bias outwards and/or reduce compression of the sleeve in an injection mode and/or post-injection mode.

There is additionally provided a medicament injector according to yet another aspect. The medicament injector includes an applicator. The medicament injector includes an interchangeable pod or cartridge assembly shaped to be insertable within the applicator. The pod or cartridge assembly is configured to receive or include a cartridge (and/or syringe and needle assembly) thereof shaped to receive or contain a medicament therewithin. Alternatively, the pod or cartridge assembly may not include but may be shaped to receive the cartridge (and/or syringe and needle assembly). The medicament injector includes a guiding mechanism configured to enable the cartridge (and/or syringe and needle assembly) thereof to move axially during an injection mode. The guiding mechanism is configured to temporarily lock in place adjacent a distal end of the pod or cartridge assembly during an injection mode when the pod or cartridge assembly is inserted in the applicator.

There is additionally provided a medicament injector according to yet another aspect. The medicament injector includes an applicator. The medicament injector includes an interchangeable pod or cartridge assembly shaped to be insertable within the applicator. The pod or cartridge assembly is configured to receive or include a cartridge (and/or syringe and needle assembly) thereof shaped to receive or contain a medicament therewithin. Alternatively, the pod or cartridge assembly may not include but may be shaped to receive the cartridge (and/or syringe and needle assembly). The medicament injector includes a guiding mechanism configured to enable the cartridge (and/or syringe and needle assembly) thereof to move axially during an injection mode. The guiding mechanism is configured to permanently lock in place adjacent a proximal end of the pod or cartridge assembly upon/during the pod or cartridge assembly being removed from the applicator thereafter.

There is further provided a medicament injector according to another aspect. The medicament injector includes an applicator. The medicament injector includes an interchangeable pod or cartridge assembly shaped to be insertable within the applicator. The pod or cartridge assembly is configured to receive or include a cartridge (and/or syringe and needle assembly) thereof shaped to receive or contain a medicament therewithin. Alternatively, the pod or cartridge assembly may not include but may be shaped to receive the cartridge (and/or syringe and needle assembly). The medicament injector includes a drive element comprising two or more telescoping members. There may be three or more or four or more telescoping members in other non-limiting embodiments. The telescoping members are movable from a retracted or nested position in a pre-injection mode, to an extended position in an injection mode.

There is additionally provided a medicament injector according to a further aspect. The medicament injector includes an applicator. The medicament injector includes an interchangeable pod or cartridge assembly having an injection end and an insertion end via which the pod or cartridge assembly is insertable into the applicator. The pod or cartridge assembly includes a cartridge (and/or syringe and needle assembly) thereof shaped to receive or contain a medicament therewithin. Alternatively, the pod or cartridge assembly may not include but may be shaped to receive the cartridge (and/or syringe and needle assembly). The cartridge (and/or syringe and needle assembly) thereof is positioned towards or closer to the insertion end of the pod or cartridge assembly when the pod or cartridge assembly is inserted into the applicator. The cartridge (and/or syringe and needle assembly) thereof is moveable towards or closer to the injection end when the medicament injector is an injection mode.

There is further provided a medicament injector according to another aspect. The medicament injector includes an applicator. The applicator includes at least one first recessed portion and at least one second recessed portion axially spaced from the at least one first recessed portion thereof. The medicament injector includes an interchangeable pod or cartridge assembly with a cartridge (and/or syringe and needle assembly) shaped to receive or contain a medicament therewithin. Alternatively, the pod or cartridge assembly may not include but may be shaped to receive the cartridge (and/or syringe and needle assembly). The pod or cartridge assembly includes at least one first latch member shaped to fit within the at least one first recessed portion and inhibit axial movement of the pod or cartridge assembly relative to the applicator in a pre-injection mode. The pod or cartridge assembly includes at least one second latch member shaped to inhibit axial movement of the cartridge (and/or syringe and needle assembly) relative to the pod or cartridge assembly in the pre-injection mode. The at least one second latch member is configured to fit at least in part within the at least one second recessed portion so as to enable axial movement of the cartridge (and/or syringe and needle assembly) in an injection mode.

There is yet also provided a medicament injector according to another aspect. The medicament injector includes an interchangeable pod or cartridge assembly with a double-ended needle unit, a cartridge shaped to receive or contain a medicament therewithin, and a telescoping mechanism. Alternatively, the pod or cartridge assembly may not include but may be shaped to receive the cartridge (and/or syringe and needle assembly). The medicament injector includes an applicator shaped to receive the pod or cartridge assembly and actuate the telescoping mechanism thereby to cause the double-ended needle unit to couple to and pierce the cartridge.

There is additionally provided a medicament injector according to another aspect. The medicament injector includes an interchangeable pod or cartridge assembly with a needle assembly. The pod or cartridge assembly includes a first telescoping mechanism. The pod or cartridge assembly includes or is shaped to receive a medicament-containing cartridge (and/or syringe and needle assembly). The pod or cartridge assembly includes an applicator shaped to receive the pod or cartridge assembly and actuate the first telescoping mechanism thereby to cause the needle assembly to pierce the cartridge. The applicator includes a second telescoping mechanism actuation of which thereafter promotes outputting of the medicament from the needle.

There is also provided a medicament injector according to another aspect. The medicament injector includes an applicator. The medicament injector includes an interchangeable pod or cartridge assembly loadable into the applicator so as to couple thereto. The medicament injector includes a medicament outputting means which automatically outputs a medicament from the pod or cartridge assembly upon subsequent retraction of the pod or cartridge relative to the applicator. The medicament injector includes an ejection means which automatically ejects the pod or cartridge assembly from the applicator after outputting of the medicament from the pod or cartridge assembly.

There is further provided a medicament injector according to a further aspect. The medicament injector includes an applicator. The medicament injector includes an interchangeable pod having an injection end, having an insertion end via which the pod is insertable into the applicator and including a cartridge (and/or syringe and needle assembly) shaped to receive or contain a medicament therewithin. The cartridge (and/or syringe and needle assembly) is positioned towards or closer to the insertion end when the pod is inserted into the applicator. The cartridge (and/or syringe and needle assembly) is moveable and/or positioned towards or closer to the injection end when the medicament injector is an injection mode.

There is also provided a barrier member and/or pod cap assembly for a medicament injector as herein described.

There is additionally provided an interchangeable pod or cartridge assembly for a medicament injector as herein described.

It is emphasized that the invention relates to all combinations of the above features, even if these are recited in different claims.

Further aspects and example embodiments are illustrated in the accompanying drawings and/or described in the following description.

### BRIEF DESCRIPTION OF DRAWINGS

The accompanying drawings illustrate non-limiting example embodiments of the invention.

FIG. 1 is a front end, top, side perspective view of a medicament injector according to one aspect, the medicament injector including an applicator, an interchangeable pod shown received within and coupled to the applicator, and a pod cap assembly coupled to the pod.

FIG. 2 is an exploded side, rear end perspective view of the medicament injector thereof, with the pod shown spaced from the applicator, with the pod and with the pod cap assembly shown coupled to the pod;

FIG. 3 is a top, side, proximal end perspective view of the pod thereof;

FIG. 4 is a first longitudinal sectional view taken along lines 4-4 of the pod of FIG. 3;

FIG. 5 is a first longitudinal sectional view thereof, with the pod shown in the process of being inserted into the applicator.

FIG. 6 is an enlarged top perspective view of the pod cap assembly, pod and applicator of FIG. 1, with the pod and applicator shown in fragment, with the pod shown in the process of being inserted into the applicator, and with an outer sleeve of the pod cap assembly being partially not shown so as to reveal a locking mechanism of the pod cap assembly, the locking mechanism comprising one or more female members and one or more male members shown received within the one or more female members thereof;

FIG. 7 is an enlarged first longitudinal sectional view of the pod cap assembly and pod and applicator of FIG. 6, with the pod shown further in the process of being inserted into the applicator;

FIG. 8 is a first longitudinal sectional view of the pod shown substantially fully inserted within the applicator of FIG. 1, with the pod cap assembly shown coupled to the pod;

FIG. 9 is an enlarged top perspective view of the pod cap assembly, pod and applicator of FIG. 1, with the pod and applicator shown in fragment, with the pod shown in the substantially fully inserted within the applicator, with the outer sleeve of the pod cap assembly being partially not shown so as to reveal the one or more male members of the locking mechanism of the pod cap assembly shown in the process of being dislodged from the female members of the locking mechanism via deflecting members of the applicator;

FIG. 10 is an enlarged, exploded top plan view of the pod cap assembly removed from the pod of FIG. 9, with the outer sleeve of the pod cap assembly being partially shown transparent so as to reveal the locking mechanism of the pod cap assembly, and with the pod and applicator being shown in fragment;

FIG. 11 is a first longitudinal sectional, exploded view of the medicament injector of FIG. 1 together with the pod cap assembly shown removed therefrom and the medicament injector being shown in an unlocked protected needle pre-injection mode;

FIG. 12 is a front or needle end, top, side perspective view of the medicament injector of FIG. 11 with the pod cap assembly removed and not shown;

FIG. 13 is a first longitudinal sectional view of the medicament injector thereof, together with a person shown in fragment, with the pod shown in the process of retracting within the applicator towards an injection mode and shown in the process of abutting against skin of the person;

FIG. 14 is a first longitudinal sectional view of the medicament injector and person of FIG. 13, with the medicament injector now shown in the injection mode thereof with a needle thereof shown fully extended and inserted within the person;

FIG. 15 is a first longitudinal sectional view of the medicament injector and person of FIG. 14, with a plunger of the applicator shown fully extended within a barrel of a cartridge (and/or syringe and needle assembly) of the pod so as to direct medicament therewithin into the person;

FIG. 16 is a first longitudinal sectional view of the medicament injector thereof shown in fragment, with the medicament injector being in a post-injection mode with the cartridge (and/or syringe and needle assembly) being retracted within the pod and the now used/depleted pod being shown in the process of being removed from the applicator;

FIG. 17 is a first longitudinal exploded view of the medicament injector thereof, with the used/depleted pod together with the applicator spaced-apart therefrom post-injection mode;

FIG. 18 is an exploded, first longitudinal sectional elevation view of a medicament injector according to another aspect, the medicament injector including an applicator and a pod shaped to be received within the applicator, the pod further including an outer cap, an inner cap, a double-ended needle unit, and a cartridge with medicament therewithin;

FIG. 19 is an inner end, side, top perspective view of the outer cap thereof;

FIG. 20 is an inner end, side, top perspective view of the inner cap of FIG. 18;

FIG. 21 is a top plan view of the outer cap of FIG. 19 with portions thereof shown removed to show the inner cap of FIG. 20 shown received therein and coupled thereto in an extended position thereof;

FIG. 22 is a first longitudinal sectional view of the pod cap assembly of FIG. 21 shown coupled to the pod, which is in the process of being inserted into the applicator;

FIG. 23 is a first longitudinal sectional view of the pod cap assembly of FIG. 21 shown coupled to the pod, which is in the process of being further inserted into the applicator;

FIG. 24 is a first longitudinal sectional view of the pod cap assembly thereof, with the inner cap shown in a retracted position, with the pod shown coupled to the applicator in an unlocked protected needle pre-injection mode, and with the double-ended needle unit of the medicament injector shown piercing the cartridge within the pod of the medicament injector;

FIG. 25 is a second longitudinal sectional view perpendicular to the first longitudinal sectional view of the pod cap assembly of FIG. 23, with the pod cap assembly shown in the process of being removed from the pod, with the inner cap shown in its extended position and including latch members thereof coupling to catches of the outer cap of the pod cap assembly so as to also facilitate removal thereby of the inner cap as well;

FIG. 26 is a first longitudinal sectional of the pod cap assembly of FIG. 25 shown removed from the pod and applicator of FIG. 25, with the medicament injector in the unlocked protected needle pre-injection mode;

FIG. 27 is an exploded, side perspective view of a medicament injector according to a further aspect, the medicament injector including an applicator shown in fragment, a pod insertable within the applicator, the pod including or being shaped to receive a pod cap assembly, a needle assembly, a user-loadable medicament-containing cartridge positioned within the pod and not seen, a rearward barrier member with cartridge-receiving arms and a coupling mechanism with cartridge-retaining arms;

FIG. 28 is an enlarged, rear end, side perspective exploded view of the pod cap assembly of FIG. 27;

FIG. 29A is a first longitudinal sectional view of the needle assembly of FIG. 27, the needle assembly including an outer hub, an inner hub telescopically coupled to and in an extended position relative to the outer hub, and a needle, with the needle being shown in fragment;

FIG. 29B is a rear end perspective view of the outer hub thereof;

FIG. 29C is a rear end, side perspective view of the outer hub thereof;

FIG. 29D is a side, rear end perspective view of the inner hub thereof;

FIG. 30A is a first longitudinal sectional view of a forward end portion of the pod of FIG. 27, with the rest of the pod shown in fragment;

FIG. 30B is a top perspective view of the forward end portion of the pod thereof, with the rest of the pod shown in fragment and with one of a pair of catch arms of the pod being shown;

FIG. 30C is a first longitudinal sectional view of an intermediate portion of the pod thereof, with the rest of the pod shown in fragment;

FIG. 30D is a first longitudinal sectional view of a rear portion of the pod thereof, with the rest of the pod shown in fragment;

FIG. 31A is a first longitudinal sectional perspective view of a sleeve of a second coupling mechanism of the medicament injector of FIG. 27, together with brackets and a rearward barrier member coupled thereto, with the rearward barrier member including a cartridge-receiving arm;

FIG. 31B is a top perspective view of the brackets thereof;

FIG. 31C is a bottom, rear end perspective exploded view of the sleeve, rearward barrier member and cartridge-receiving arms of FIG. 31A in the process of being inserted into the rear portion of the pod, with the rest of the pod being shown in fragment;

FIG. 32A is side, bottom perspective view of a plurality of cartridge-retaining arms of the second coupling mechanism of the medicament injector of FIG. 27, together with outer arms which function to deflect radially inwards the cartridge-receiving arms of FIG. 31C when the pod of FIG. 27 is inserted into the applicator of FIG. 27;

FIG. 32B is a bottom, side perspective view thereof;

FIG. 33A is a first longitudinal sectional view of the pod of FIG. 27 shown in assembled form, with the pod cap assembly, needle assembly, rearward barrier member with cartridge-receiving arms and coupling mechanism with cartridge-retaining arms shown coupled to and/or inserted within a housing thereof;

FIG. 33B is a second longitudinal sectional view perpendicular to the first longitudinal sectional view of the pod so assembled of FIG. 33A;

FIG. 34 is a second longitudinal sectional view thereof, with a cartridge of the medicament injector of FIG. 27 shown in the process of being inserted into the pod via the cartridge-receiving arms of the pod;

FIG. 35 is a second longitudinal sectional view of the pod of FIG. 34 together with the applicator of FIG. 27, with the cartridge shown fully inserted within the pod, with an inner hub of the needle assembly shown extending about, receiving and coupling to a cartridge cap of the cartridge, and with the pod being shown in the process of being inserted into the applicator;

FIG. 36A is a first longitudinal sectional view of the pod thereof shown in the process of coupling to the applicator thereof, with the pod shown just prior to snap fit coupling to the applicator via a first coupling mechanism thereof;

FIG. 36B is a second longitudinal sectional view thereof;

FIG. 37 is a second longitudinal sectional view thereof with the pod now fully inserted into and coupling with the applicator, with the outer hub of the needle assembly extending about, receiving and operatively connecting to the inner hub of the needle assembly and cap of the cartridge so as to enable a needle of the needle assembly to pierce of the cartridge and access medicament therewithin, and with the pod cap assembly now being in an unlocked position and the medicament injector being in an unlocked protected needle pre-injection mode;

FIG. 38 is a first longitudinal sectional view thereof, with the pod coupled to the applicator via the first coupling mechanism thereof and with the unlocked pod cap assembly being shown in the process of being removed from the forward end portion of the pod, and with the medicament injector being in the unlocked protected needle pre-injection mode;

FIG. 39 is a first longitudinal sectional view thereof, together with a person shown in fragment, with the forward end portion of the pod shown in the process of pressing/abutting against the skin of a person so as to cause retraction of the pod relative to and into the applicator and so as to decouple the second coupling mechanism so as to move the pod from the unlocked protected needle pre-injection mode of FIG. 38 to an injection mode which enables deployment of the needle assembly outwards of the applicator;

FIG. 40A is a first longitudinal sectional view thereof, with the forward motion of the pod relative to the applicator showing the needle in a deployed position with the medicament injector shown in the injection mode;

FIG. 40B is a second longitudinal sectional view thereof, with the forward motion of the pod relative to the applicator enabling the cartridge-receiving arms to move forward and extend radially outwards into recessed portions of the pod so as to enable passage of a plunger of the applicator therethrough so as to abut a piston of the cartridge;

FIG. 41A is a first longitudinal sectional view thereof, with the plunger shown fully extended so as to fully output medicament from the cartridge and into the person;

FIG. 41B is a second longitudinal sectional view thereof;

FIG. 42A is an enlarged sectional view of the forward end portion of the pod shown in fragment, with the pod being shown in the process of extending outwards of the applicator to a fully needle deployed position, with the outer hub of the needle assembly including a pair of prongs slidably abutting past which the catch arms of the pod FIG. 30B;

FIG. 42B is an enlarged sectional view thereof, with the pod being shown in a fully needle deployed position and in which the prongs are facing and abut the catch arms so as to inhibit inadvertent retraction of the pod relative to the applicator during the process of injecting medicament outwards of the cartridge;

FIG. 42C is an enlarged sectional view thereof, with the plunger of the applicator shown fully extending through the barrel so as to fully output medicament from the cartridge; and

FIG. 42D is an enlarged sectional view thereof, with the pod shown removed from and no longer abutting the person, which enables the pod to spring-bias outwards from the applicator in part, which enables the catch arms to radially outwardly bias from the pod, which causes the prongs to dislodge therefrom and which enables the forward end portion of the pod to move forward so as to extend about the needle assembly anew;

FIG. 43A is a first longitudinal sectional view of the pod and applicator of FIG. 35 in a post-injection mode, with the cartridge-depleted pod shown automatically ejecting from the applicator so as to enable the applicator to receive another cartridge-containing pod; and

FIG. 43B is a second longitudinal sectional view of the pod and depleted cartridge of FIG. 43A.

### DESCRIPTION OF THE PREFERRED EMBODIMENTS

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

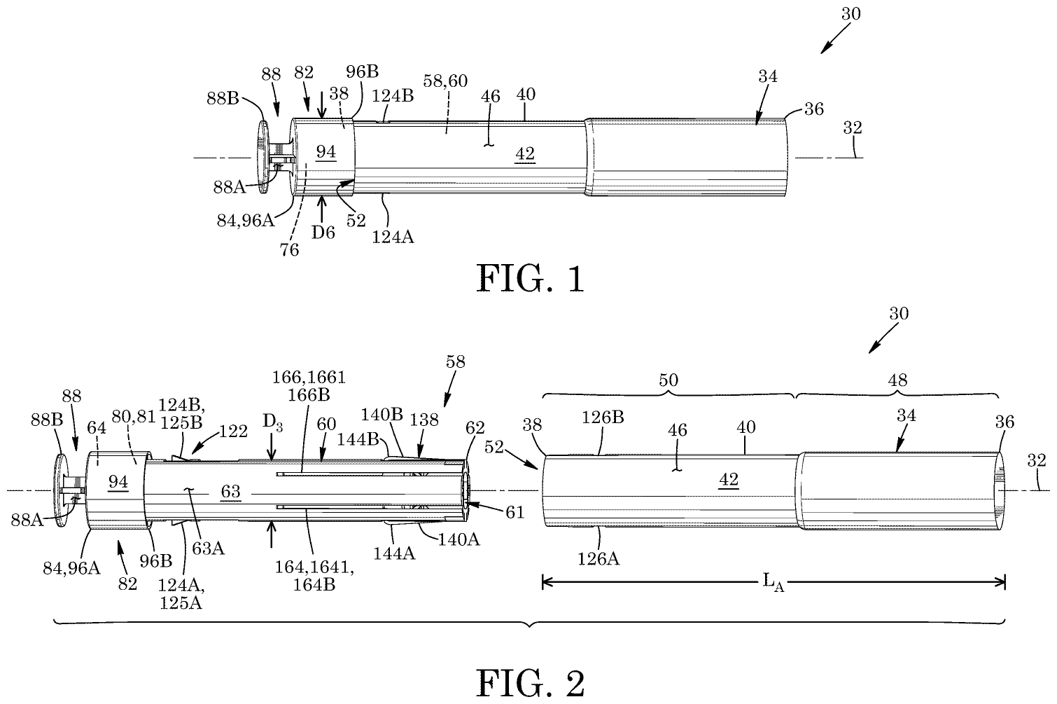

Referring to the drawings and first to FIG. 1, there is shown a medicament injector 30. The medicament injector is elongate and extends along a longitudinal axis 32. Medicament injector 30 is generally tubular and cylindrical in outer shape in this non-limiting embodiment; however, this is not strictly required.

Still referring to FIG. 1, the medicament injector includes an applicator 34. As seen in FIG. 2, the applicator has a first, closed, plunger or proximal end 36 and a second, open, needle, injection, patient or distal end 38 longitudinally spaced from the proximal end thereof. Applicator 34 has a length L_{A} extending between the proximal and distal ends thereof.

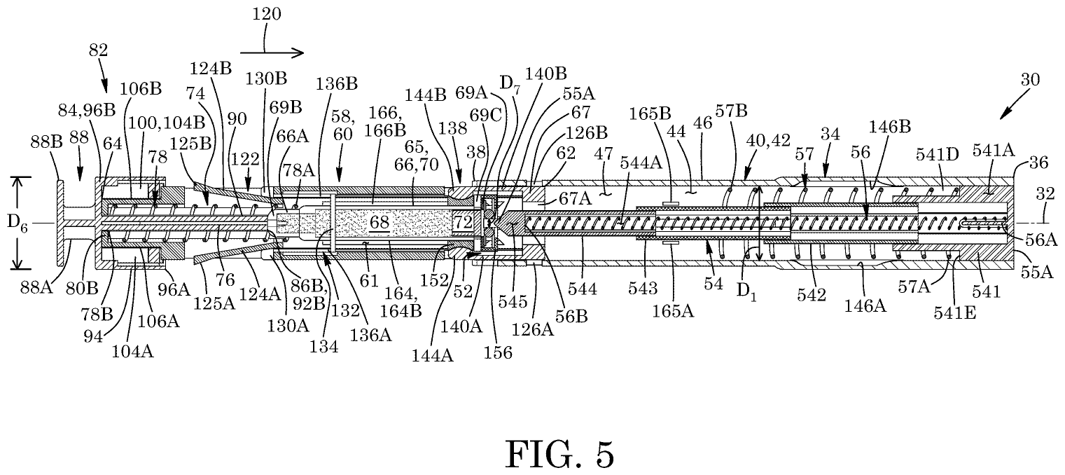

The applicator includes an elongate housing or hollow outer body 40 that extends between ends 36 and 38 thereof. As seen in FIG. 5, the housing is tubular in this non-limiting embodiment. Outer body 40 of applicator 34 includes a wall 42, which may be referred to as an outer wall of applicator 34. The wall has an inner surface 44 and an outer surface 46 radially spaced from the inner surface thereof. Applicator 34 has an interior 47 about which wall 42 extends. The interior of the applicator is in fluid communication with inner surface 44 of the wall of the applicator.

As seen in FIG. 2, outer body 40 of applicator 34 in this non-limiting embodiment includes a first or proximal portion 48 that extends from proximal end 36 towards distal end 38 of the applicator. The outer body of the applicator includes a second or distal end portion 50 that extends from the distal end towards the proximal end of the applicator. As seen in FIG. 17, wall 42 along proximal end portion 48 of the outer body of the applicator is generally thicker than along the distal end portion of the outer body in this non-limiting example; however, this is not strictly required.

Still referring to FIG. 17, applicator 34 has a passageway, in this example a receptacle 52. The receptacle may be referred to as a cartridge-accepting receptacle, a cartridge receptacle, a pod-accepting receptacle or a pod receptacle, for example. The receptacle is defined by outer body 40 in this example and extends from distal end 38 towards proximal end 36 of the outer body. Alternatively, receptacle 52 may be said to be located at a front or front end of the applicator. The receptacle 52 has a diameter D₁, which may be referred to as an inner diameter of outer body 40 of applicator 34.

Applicator 34 includes a drive element, in this non-limiting embodiment a drive element configured to store potential energy, with the drive element in this non-limiting example being spring-biased outwards and comprising a plunger 54. However, this is not strictly required and the drive element may comprise other types of drive elements and/or energy/power sources in other embodiments, such as including a gas cartridge and/or hydraulic power, a battery and/or electrical power etc. Plunger 54 has a first or proximal end 55A adjacent proximal end 36 of applicator 34, and a second or distal end 55B axially spaced-apart form the proximal end thereof. The plunger in this non-limiting example includes two or more and in this example four telescoping members: a first, outer or proximal telescoping member 541, a second, middle or proximal intermediate telescoping member 542, a third, middle distal intermediate telescoping member 543, and a fourth, inner or distal telescoping member 544. However, this number of telescoping members is not strictly required and there may be three members or greater than four members in other embodiments. Each telescoping member is tubular in this example. Plunger 54 may thus be referred to as a multi-telescoping plunger. The plunger may be referred to as a telescoping mechanism of medicament injector 30.

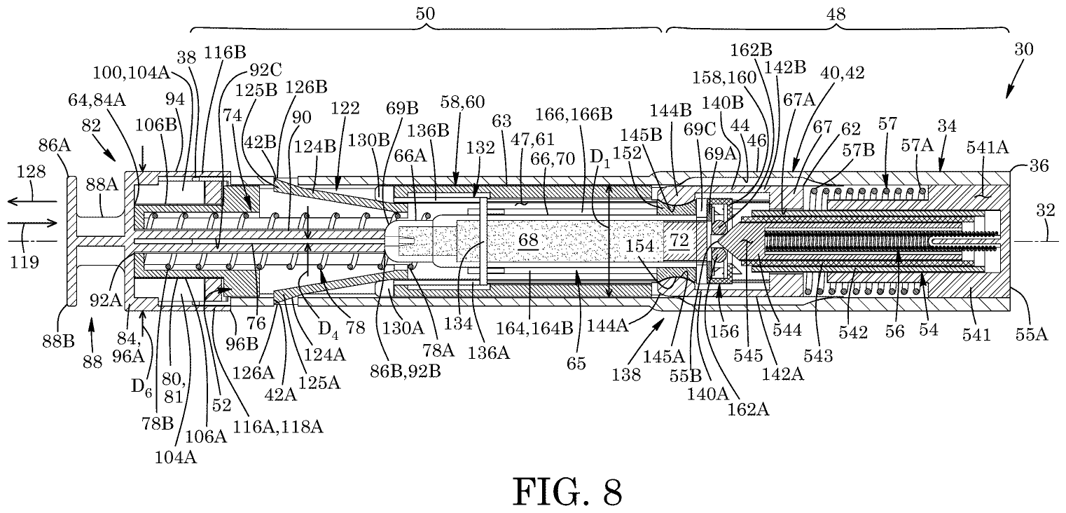

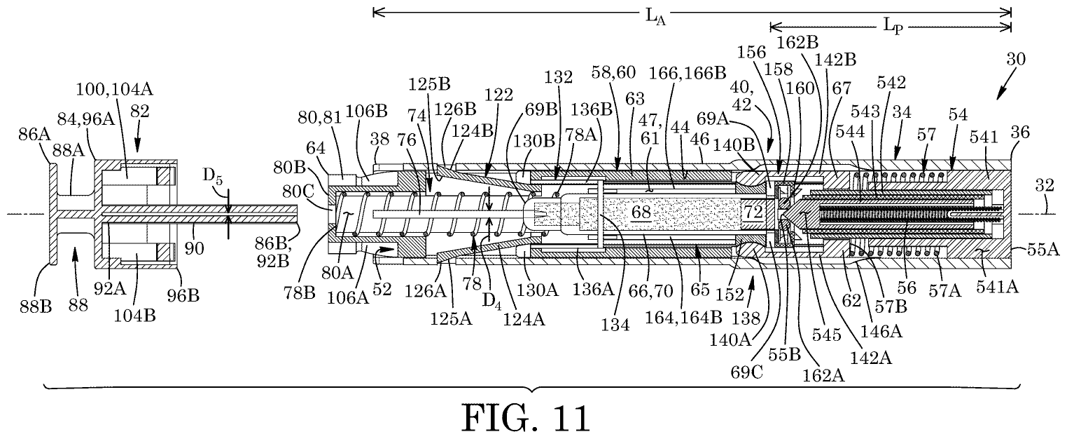

Telescoping members 541, 542, 543 and 544 have a primed, retracted, rearward or nested position seen in FIG. 8 in a locked protected needle pre-injection mode of medicament injector 30. In the nested position, telescoping member 544 is shaped to be received at least in part within telescoping member 543, which is shaped to be received at least in part within telescoping member 542, which is shaped to be received at least in part within telescoping member 541. Plunger 54 (or telescoping member 544 thereof) includes a distal end portion 545 that is outwardly tapered in this non-limiting embodiment, in this example being triangular in lateral cross-section. The distal end portion of the plunger extends from distal end 55B towards proximal end 55A of the plunger. Distal end portion 545 of plunger 54 may be referred to as a plunger tip. The plunger in its retracted/nested position has a length L_{P} seen in FIG. 11 extend between ends 55A and 55B thereof. The length of plunger 54 in its nested or retracted position is equal to or less than one third of length L_{A} of applicator 34 in this non-limiting example; however, this is not strictly required.

Proximal telescoping member 541 couples to outer body 40 of applicator 34. The proximal telescoping member includes in this non-limiting example a base 541A that is cylindrical in outer shape. The base of proximal telescoping member 541 in this non-limiting example snugly and/or interference fits within and couples to outer body 40 of applicator 34. The base extends from adjacent proximal end 36 towards distal end 38 of the outer body of the applicator in this non-limiting embodiment.

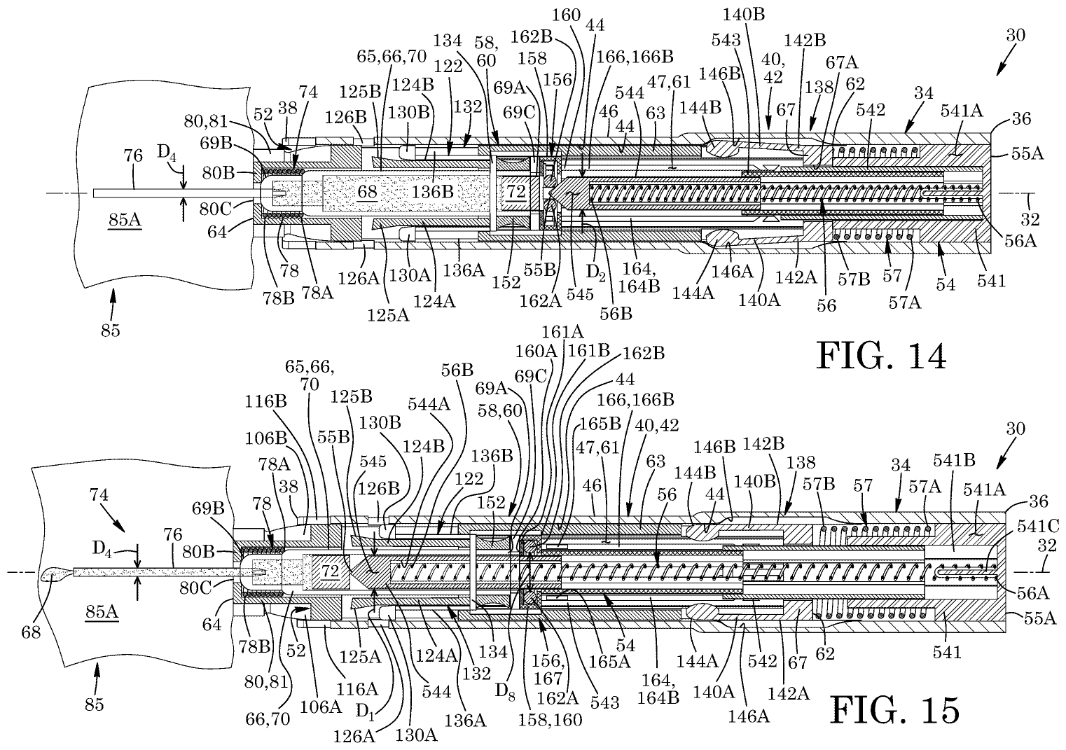

Plunger 54 is moveable from the retracted position seen in FIG. 8, to an extended or forward position seen in FIG. 15 in the injection mode of the medicament injector. Still referring to FIG. 15, telescoping members 541, 542, 543 and 544 are configured to bias axially outwards towards distal end 38 of applicator 34 via a first resilient member, in this example a first or inner coil spring 56. The drive element may thus be said to comprise spring-biased telescoping members. Inner coil spring 56 is configured to be extended/uncompressed at least in part in the extended position of telescoping members 541, 542, 543 and 544 seen in FIG. 5. The inner coil spring is configured to store potential energy, in this example spring potential energy, via the action of moving the telescoping members to their compressed/primed/nested position seen in FIG. 8, which causes the spring to also compress and be primed. Inner coil spring 56 has a first or proximal end portion 56A which couples to and/or abuts against proximal telescoping member 541. The proximal end portion of inner coil spring 56 in this non-limiting example is received within a bore 541B of base 541A of proximal telescoping member 541 and extends about an axially-extending and coaxially positioned protrusion or stem 541C of the proximal telescoping member seen in FIG. 15. The inner coil spring has a distal end portion 56B longitudinally spaced from proximal end portion 56A thereof. The distal end portion of inner coil spring 56 couples to and/or abuts against distal telescoping member 544: in this non-limiting example extending within elongate and coaxial bore 544A of the distal telescoping member. As seen in FIG. 14, the distal telescoping member has a cross-sectional diameter D₂.

Referring now to FIG. 5, applicator 34 includes a second resilient member, in this example a second or outer coil spring 57. The outer coil spring has a first or proximal end portion 57A which couples to and/or abuts proximal telescoping member 541. Spring 57 in this non-limiting example is received within an outer, annular recessed portion 541D of proximal telescoping member 541 and abuts a radially-extending shoulder 541E of base 541A of the proximal telescoping member. The outer coil spring has a distal end portion 57B longitudinally spaced from the proximal end portion 57A.

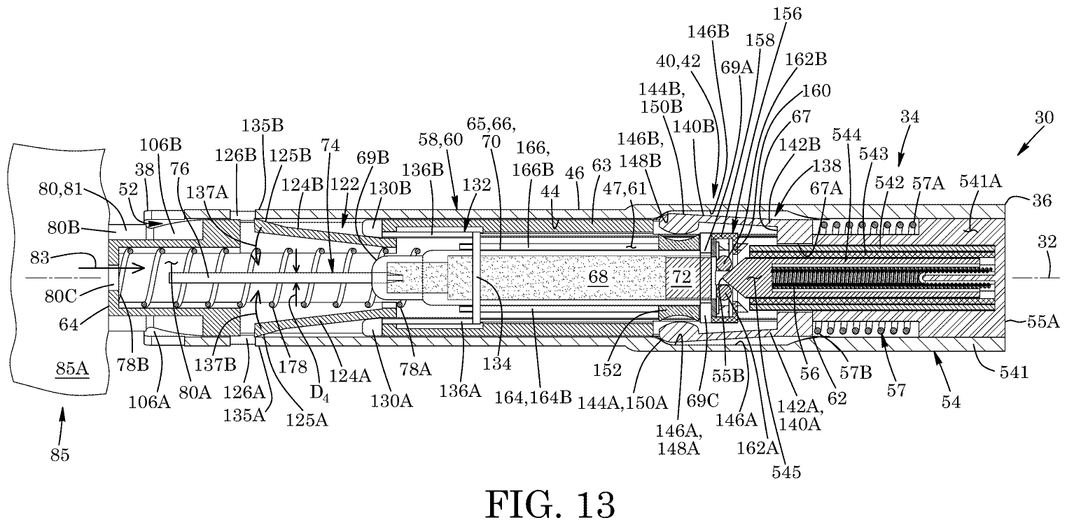

As seen in FIG. 2, medicament injector 30 includes an interchangeable pod 58. Referring to FIGS. 13, 15 and 17, the pod is loadable into the applicator so as to couple thereto and prime plunger 54, with subsequent retraction of the pod actuating/releasing/firing plunger 54 to engage the pod so as to output medicament 68 therefrom and with the applicator being configured to automatically eject the pod from the receptacle thereafter. The applicator is inhibited from contacting fluid medicament, with all medicament contacting and/or fluid path components of medicament injector 30 (e.g. those relating to the pod) being automatically ejected from receptacle 52 following administration of the medicament. Upon pod 58 being ejected, plunger 54 is configured to receive another pod and be primed anew, with applicator 34 therefore being configured to be reusable. Thus, upon the pod being ejected, the plunger is configured to return to a native state thereof to receive a subsequent said pod. The following is a non-limiting embodiment which achieves the above set out functionality.

Pod 58 is elongate and extends along axis 32. The pod is configured to be coaxial with applicator 34 in this non-limiting example when the pod and applicator are coupled together. The pod includes an outer body or housing or a pod housing 60. The pod housing is substantially cylindrical in outer profile in this non-limiting example; however, this is not strictly required. Pod housing 60 encloses an interior 61. Pod 58 has a first, insertion, or proximal end 62 and a second, needle, injection, patient or distal end 64 longitudinally spaced from the proximal end thereof. Pod housing 60 extends between the ends of the pod and includes a wall 63 that extends about interior 61 thereof. The wall of the pod housing has an exterior surface 63A. Pod 58 has a laterally/radially-extending outer diameter D₃.

Pod 58 is shaped to be received within applicator 34, in this example receptacle 52 of the applicator, via proximal end 62 of the pod. Medicament injector 30 may thus be referred to as a front-loaded medicament injector in this non-limiting embodiment; however this is not strictly required. The medicament injector may be rear-loading or a rear-loaded medicament injector in other embodiments. Pod 58 is configured to be snugly received within receptacle 52 of applicator 34 in this non-limiting embodiment. Referring FIG. 17, diameter D₃ of the pod is substantially equal to or slightly smaller than diameter D₁ of receptacle 52.

As seen in FIG. 8, pod 58 is configured to be spring-biased forward by applicator 34, in this example via outer coil spring 57. The outer coil spring is shaped via distal end 57B thereof to operatively abut proximal end 62 of the pod when the pod is inserted within the applicator. In this non-limiting embodiment, pod 58 includes a first or rear end portion, base or insert 67 to which the distal end of the spring abuts. The insert of pod 58 is annular in this non-limiting embodiment, with a bore 67A, coaxial with axis 32, extending therethrough. The bore is sized to enable members 542, 543 and 544 of plunger 54 to extend therethrough.

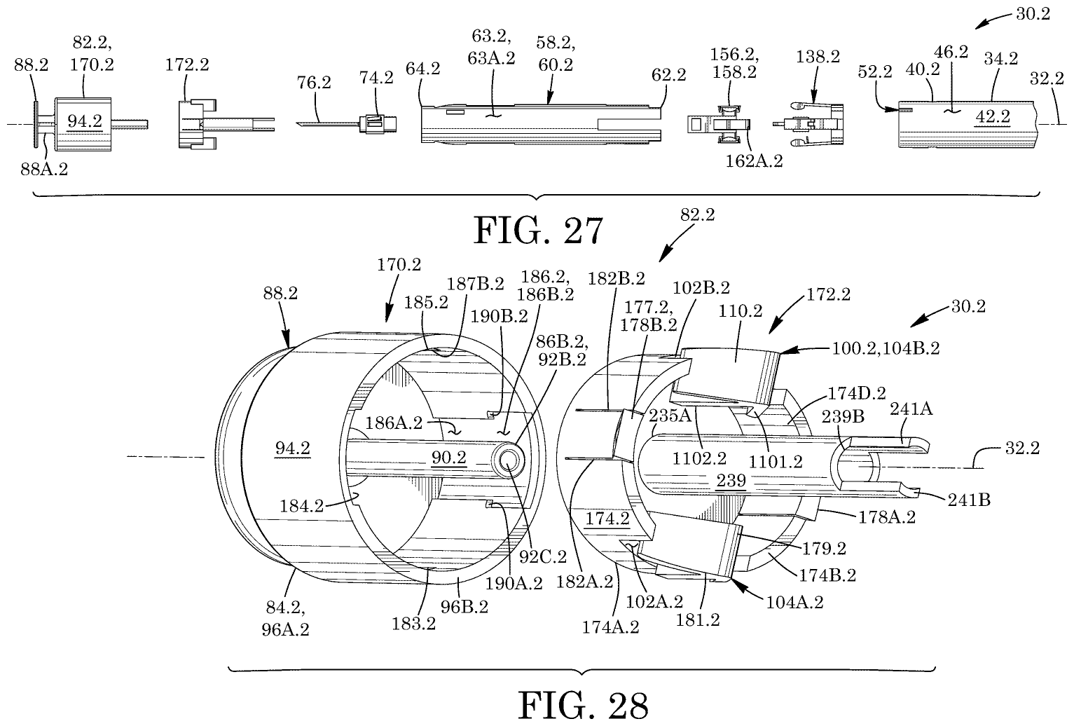

Referring now to FIG. 5, pod 58 includes in this non-limiting embodiment an interchangeable cartridge 65. However, this is not strictly required and in other embodiments the pod may not include a cartridge and may be configured/shaped to receive a user-insertable and/or third party cartridge as well be discussed in further detail for the embodiment shown in FIGS. 27 to 43B.

Referring back to FIG. 5, cartridge 65 includes a hollow body, in this non-limiting example a barrel 66 shaped to receive or contain a medicament 68 therewithin. The cartridge may thus be referred to as a medicament-containing cartridge. Barrel 66 has a first, proximal, closed or rear end 69A and a second, distal, open or forward end 69B axially spaced from the rear end thereof. The barrel includes in this non-limiting example an annular flange 69C which extends radially outwards and which is adjacent the rear end thereof.

Medicament injector 30 may be said to include a syringe assembly 70 in this non-limiting embodiment, with cartridge 65 and barrel 66 being a part of the syringe assembly in this non-limiting example; however, this is not strictly required. Rear end 69A of the barrel may be referred to as the rear of cartridge 65 and/or the syringe assembly. Syringe assembly 70 is elongate and is coaxial with pod 58 and applicator 34 in this non-limiting embodiment. Pod housing 60 is shaped to receive cartridge 65 and syringe assembly 70 therewithin. Pod 58 is thus configured to receive and/or include the cartridge and/or syringe assembly in this non-limiting embodiment.

Cartridge 65 (and syringe assembly 70) includes a piston 72 sealably positioned within and slidably coupled to barrel 66. The piston is adjacent rear end 69A of the barrel when the barrel is filled with medicament 68 in this non-limiting example. Piston 72 may be referred to as a stopper or syringe stopper in one non-limiting embodiment. As seen in FIG. 5, cartridge 65 and/or syringe assembly 70 are configured to be positioned towards or closer to proximal end 62 of pod 58 when the pod is inserted into applicator 34 in this non-limiting example.

As seen in FIG. 15, plunger 54 in the extended position thereof is configured to selectively extend through barrel 66 thereof via rear end 69A. The plunger is configured to selectively abut, bias and move piston 72 from rear end 69A of barrel 66 seen in FIG. 5, to or towards forward end 69B of the barrel seen in FIG. 15. Plunger 54 of applicator 34 may thus be said to selectively extend within syringe assembly 70.

Referring to FIG. 13, pod 58 and/or the syringe assembly include a needle assembly 74 comprising a needle 76 via which medicament 68 is injectable. The needle is a staked needle in this non-limiting embodiment. Needle 76 in this non-limiting example couples to cartridge 65, in this example barrel 66 thereof, in this case via forward end 69B of the barrel. Needle 76 is elongate, coaxial with pod housing 60 and extends parallel to axis 32 in this non-limiting example. The needle is in fluid communication with medicament 68. However, this arrangement is not strictly required and the needle may couple and/or selectively couple to the rest of syringe assembly 70, cartridge 65 and/or barrel 66 thereof in other manners in other embodiments. Needle 76 has a laterally/radially-extending outer diameter D₄.

Referring to FIG. 8, syringe assembly 70 (including needle assembly 74) is biased towards a retracted position, being biased towards proximal end 62 of pod 58 in this non-limiting embodiment. The pod is resiliently biased to extend about needle 76 in an unlocked protected needle injection mode seen in FIG. 11, with retraction of the pod into applicator 34 (as seen in FIG. 13) functioning to deploy the needle as seen in FIGS. 14 and 15. The following is a non-limiting embodiment which achieves this functionality.

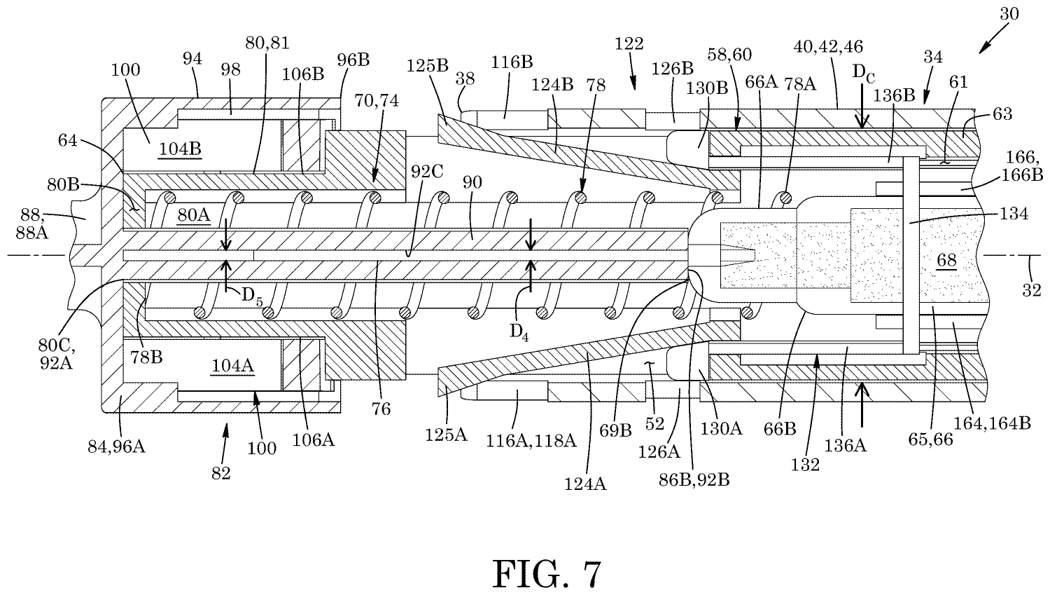

Referring to FIG. 7, pod 58 includes a resilient member, in this example a coil spring 78. The spring has a first or proximal end portion 78A which operatively connects to and/or abuts against cartridge 65 (including barrel 66 thereof): in this non-limiting example extending about neck 66A of the barrel adjacent distal end 69B of the barrel and abutting annular shoulder 66B of the barrel adjacent thereto. Spring 78 has a distal end portion 78B longitudinally spaced from the proximal end portion thereof. The distal end portion of the spring couples to and/or abuts against an axially opposite portion of pod 58: in this non-limiting example a second, distal, front or forward end portion 80 of the pod located adjacent distal end 64 of the pod. The forward end portion of the pod extends from distal end 64 towards proximal end 62 of the pod seen in FIG. 5. Referring back to FIG. 7, forward end portion 80 of pod 58 has an axially-extending bore 80A shaped to receive distal end portion 78B of spring 78 therein. The forward end portion of the pod includes a laterally/radially-extending stop, flange or seat 80B shaped to abut the distal end portion of the spring in this non-limiting embodiment. As seen in FIG. 11, the seat is positioned adjacent distal end 64 of pod 58 in this non-limiting example. Syringe assembly 70, cartridge 65 and/or barrel 66 and the needle thereof, are thus biased towards or closer to proximal end 62 of pod 58 in the pre-injection mode and injection mode seen in FIGS. 8 and 13 in this non-limiting example. Needle 76 of the syringe assembly is axially rearwardly spaced from distal end 64 of the pod in the pre-injection in this non-limiting embodiment. A central aperture 80C extends longitudinally through seat 80B and/or forward end portion 80 of the pod to selectively enable passage of needle 76 therethrough.

Referring to FIG. 13, medicament injector 30 includes a needle protection assembly 81, in this non-limiting embodiment comprising a needle sheath or sleeve, in this case comprising forward end portion 80 of pod 58 thereof. The forward end portion of the housing of the pod is configured to extend about needle 76 in the pre-injection mode and the beginning stage of the injection mode of medicament injector 30 seen in FIG. 13, with spring 78 biasing the forward end portion of the pod axially outwards. Forward end portion 80 of pod 58 may be referred to as a first sheath of needle 76. The forward end portion of the pod is moveable from the pre-injection mode partially axially rearward as seen by arrow 83 in FIG. 13 when upon pressing against or abutting skin 85A of person 85. The person may be a patient in this non-limiting embodiment. Abutting/biasing forward end portion 80 of pod 58 against person 85 functions to compress spring 78 to initiate automatic movement of medicament injector 30 to its injection mode seen in FIG. 15 in which needle 76 is exposed and as will be discussed further below.

Referring to FIG. 1, medicament injector 30 includes a first or forward barrier member, in this non-limiting example a reversible barrier member, in this non-limiting embodiment a pod cap assembly 82. The pod cap assembly may be referred to as a reversible pod cap assembly. Referring to FIG. 8, pod cap assembly 82 is configured such that removal thereof from pod 58 to enable access to medicament 68, requires the pod to be inserted within applicator 34, in this example via receptacle 52 of the applicator. The pod cap assembly is configured to inhibit access to the medicament until the pod is inserted into the applicator. Pod cap assembly 82 according to one non-limiting example is removable only upon pod 58 being substantially fully inserted into the applicator; however the latter is not strictly required in other embodiments. The pod cap assembly is removable upon the pod coupling applicator 34, in this non-limiting example upon the pod snap-fit connecting to the applicator. Pod cap assembly 82 may be shaped to inhibit inadvertent deployment of the needle when the pod is being inserted into the applicator according to another non-limiting example. The pod cap assembly according to another non-limiting embodiment is configured to inhibit access to the pod via distal end 64 of the pod until the pod is inserted within applicator 34. Pod cap assembly 82 in one embodiment is shaped to promote sterility of pod 58 and/or interior 61 thereof. The following is a non-limiting embodiment which achieves the above functionality.

As seen in FIG. 8, pod cap assembly 82 is configured in this non-limiting example to interference fit couple at least in part to forward end portion 80 of pod 58. The pod cap assembly includes a cover member 84 shaped to extend across and abut distal end 64 of the pod and the forward end portion of the pod when the pod cap assembly is coupled to the pod. The cover member is substantially laterally-extending, planar and circular in shape in this non-limiting example. Cover member 84 (including inner surface 84A thereof) when coupled to pod 58, is configured to extend across, block and/or cover central aperture 80C of forward end portion 80 of the pod so as to inhibit access to needle 76. The cover member is configured as well as to inhibit the needle from extending therethrough and/or medicament 68 from passing therethrough.

Referring to FIG. 11, pod cap assembly 82 has a first, proximal or gripping end 86A and a second, distal or sheath end 86B axially spaced from the proximal end thereof. The pod cap assembly in this non-limiting example includes a gripping member 88 shaped to facilitate gripping thereof; however, this feature may not be strictly required. The following non-limiting embodiment achieves this functionality. Gripping member 88 extends from gripping end 86A towards sheath end 86B of pod cap assembly 82. The gripping member includes an inner portion 88A which couples to and extends axially outwards from covering member 84. The inner portion of gripping member 88 is cross-shaped in lateral section in this non-limiting example; however, this is not strictly required. Gripping member 88 includes an outer portion 88B which couples to and extends laterally outwards from inner portion 88A thereof. The outer portion is planar and/or substantially circular in this non-limiting example.

Pod cap assembly 82 is shaped to extend about needle 76 in an airtight manner in this non-limiting embodiment. The following is non-limiting example that achieves this functionality. As seen in FIG. 11, pod cap assembly 82 includes a first or inner sleeve or sheath 90 shaped to receive and extend about needle 76. The inner sheath may be referred to as a stem or stem member. Inner sheath 90 is tubular in this non-limiting example. Referring to FIG. 7, inner sheath 90 is shaped to extend through central aperture 80C of end portion 80 of pod 58. The inner sheath has a first, proximal or closed end 92A and a second, distal or open end 92B axially spaced from the proximal end thereof. The distal end of inner sheath 90 corresponds to sheath end 86B of pod cap assembly 82 in this example. The inner sheath extends axially outwards to the sheath end of pod the cap assembly. Inner sheath 90 connects to cover member 84 via proximal end 92A thereof, in this example integrally connecting to the cover member so as to form a unitary whole or one contiguous piece.

The inner sheath has a passageway or bore 92C extending from open end 92B to closed end 92A thereof. The bore of inner sheath 90 is shaped to snugly receive needle 76 therethrough. In this non-limiting example bore 92C of the inner sheath has a diameter D₅ substantially equal to or slightly larger than diameter D₄ of needle 76. Inner sheath 90 of pod cap assembly 82 may be referred to as a first sheath of needle 76. The inner sheath in this non-limiting embodiment is configured to cover needle 76 in an airtight manner when pod cap assembly 82 couples thereto.

Inner sheath 90 of pod cap assembly 82 functions as a secondary/backup mechanism to inhibit accidental deployment of needle 76 as pod 58 is being inserted into applicator 34. Open end 92B of inner sheath 90 of the pod cap assembly is shaped to function as a stop which abuts neck 66A and/or forward end 69B of barrel 66 when the pod cap assembly couples to pod 58 as seen in FIG. 7. The inner sheath abuts the neck of the syringe so as to keep the inner sheath and the syringe in place while pod cap assembly 82 is locked onto pod 58.

As seen in FIG. 8, the pod cap assembly is shaped to extend about forward end portion 80 of the pod and/or distal end 38 of applicator 34. The following non-limiting embodiment achieves this functionality. Pod cap assembly 82 in this non-limiting embodiment includes a second or outer sheath or sleeve 94. The outer sleeve is tubular in this example. Outer sleeve 94 extends longitudinally and is configured to be coaxial with applicator 34 and pod 58 when coupled thereto in this non-limiting embodiment. The outer sleeve has a first, proximal or closed end 96A and a second, distal or open end 96B axially spaced from the proximal end thereof. The distal end of outer sleeve 94 is positioned between cover member 84 and sheath end 86B of pod cap assembly 82 in this non-limiting example. The outer sleeve couples to the cover member via proximal end 96A thereof and extends axially outwards/rearwards to sheath end 86B of the pod cap assembly. Outer sleeve 94 has an outer diameter D₆ that is larger than diameter D₁ of receptacle 52, thereby inhibiting pod cap assembly 82 from fully extending therewithin. End 96B of the outer sleeve of the pod cap assembly is configured to abut distal end 38 of applicator 34. The outer sleeve of pod cap assembly 82 when coupled to pod 58, extends about needle 76 at least in part in this example.

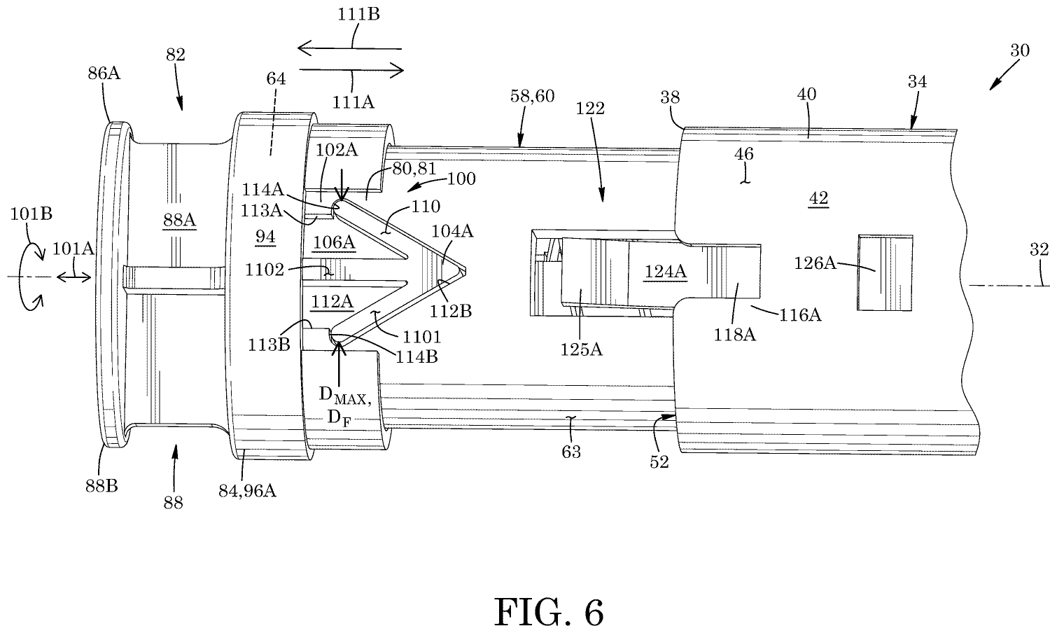

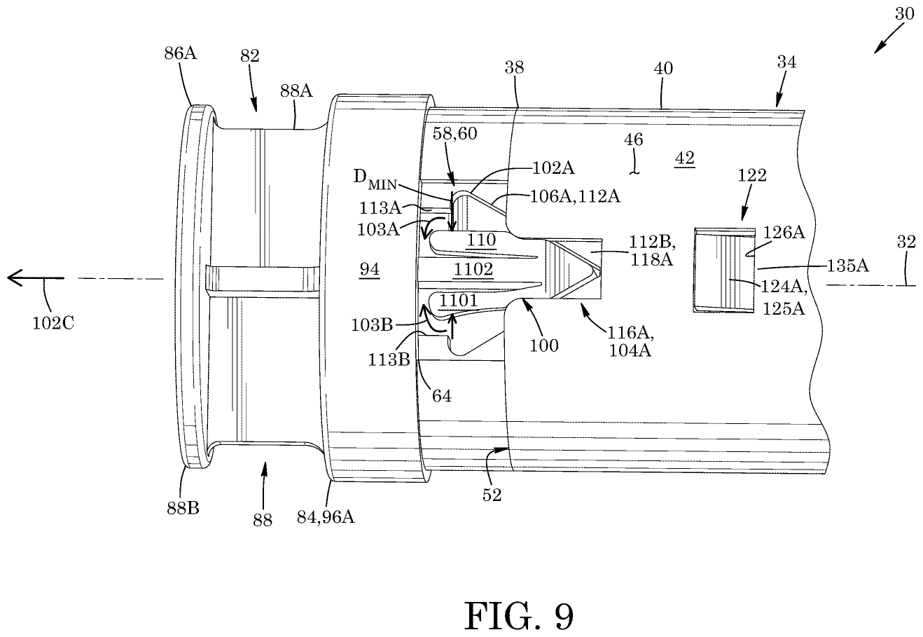

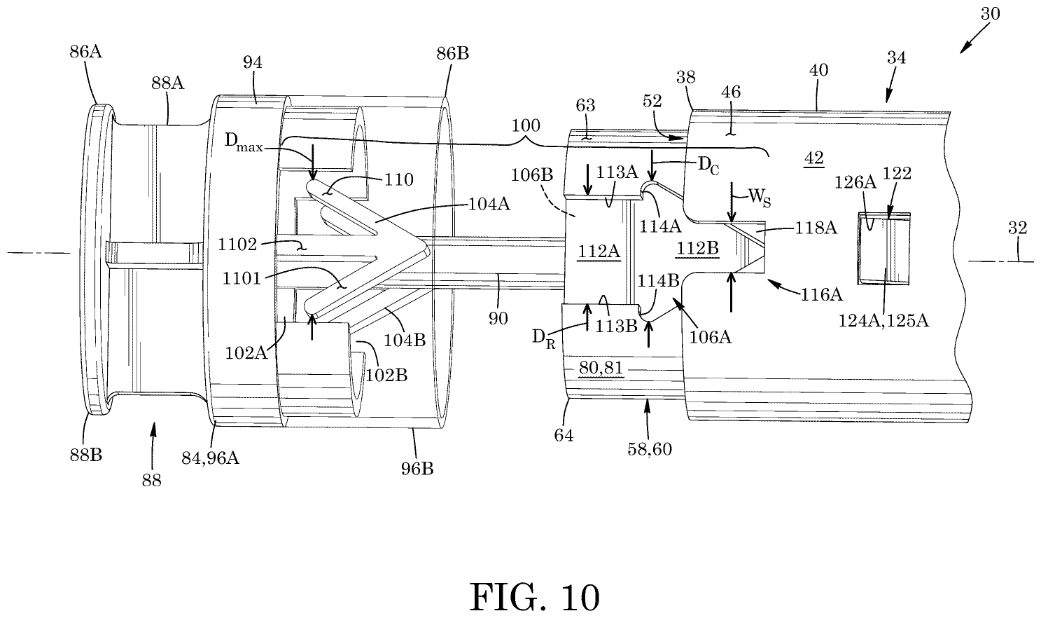

Referring now to FIGS. 9 and 10, medicament injector 30 includes a first coupling or locking mechanism via which the pod cap assembly couples to pod 58, in this example a cap coupling mechanism or cap locking mechanism 100. The cap locking mechanism is configured to inhibit removal of pod cap assembly 82 until the pod is inserted within and/or couples to applicator 34. Pod cap assembly 82 is configured to be locked in place via cap locking mechanism 100 until pod 58 is inserted into and/or couples to applicator 34. The cap locking mechanism has a locked mode seen in FIG. 6, in which axial movement (as shown by arrow 101A) and rotational movement (as shown by arrow 101B) of the pod cap assembly relative to pod 58 is inhibited. Cap locking mechanism 100 has an unlocked mode seen in FIGS. 9 and 10, in which pod cap assembly 82 is movable axially outwards (as shown by arrow 102C in FIG. 9) relative to the pod so as to remove the pod cap assembly from pod 58.

The cap locking mechanism in this non-limiting example comprises one or more male members and one or more female members shaped to receive the male members thereof, with insertion of the pod into applicator 34 as seen in FIG. 9 functioning to deform the male members in part. The following is a non-limiting embodiment which achieves this functionality. Referring to FIG. 10, pod cap assembly 82 includes at least one and in this example a pair of circumferentially spaced male members 104A and 104B. Pod 58 comprises at least one and in this example of circumferentially spaced-apart female members 106A and 106B. The male members may be referred to as latch members and/or arms or pod cap latch members and/or arms. Female members 106A and 106B are shaped to receive respective ones of the male members in the locked mode of pod cap assembly 82 as seen in FIG. 6 and this functions to inhibit removal of the pod cap assembly from the pod. Insertion of pod 58 and pod cap assembly 82 into applicator 34 as seen in FIG. 9, functions to dislodge male members 104A from corresponding female members, thereby enabling and/or causing the release of the pod cap assembly from the pod.

The following are yet further particulars of a non-limiting embodiment that achieves this functionality. As seen in FIG. 6, each male member 104A of cap locking mechanism 100 includes one or more resilient elongate protrusions, in this example a pair of elongate protrusions 110 and 1101. Each elongate protrusion in the locked position of the cap locking mechanism, extends radially/laterally outwards at least in part, in this example being angled relative to axis 32. Each male member 104A, in this example including elongate protrusions 110 and 1101 thereof so angled/shaped, is configured to facilitate insertion or movement of pod cap assembly 82 in a first or insertion direction 111A towards pod 58 and inhibit movement of the pod cap assembly in a second or removable direction 111B away from the pod thereafter. The elongate protrusions span a laterally-extending maximum distance D_{MAX} adjacent distal ends thereof. Each male member 104A thus extends longitudinally and laterally in part when in the locked position in this non-limiting embodiment. Elongate protrusions 110 and 1101 of male member 104A are angularly spaced in the locked position in this non-limiting example. Each male member is V-shaped in this non-limiting example, in this case being hook or harpoon-shaped; however this is not strictly required. Cap locking mechanism 100 may thus be referred to as a hook and/or harpoon mechanism. As seen with reference to FIG. 7, male members 104A and 104B are inwardly positioned parts of outer sleeve 94 in this non-limiting example.

As seen in FIG. 10, each male member 104A in this non-limiting example includes a connector portion 1102. As seen in FIG. 6, each connector portion extends parallel to and is radially offset from axis 32 in this non-limiting example. Each connector portion 1102 couples to outer sleeve 94 and extends towards open end 96B of the outer sleeve in this non-limiting embodiment. For each male member 104A elongate protrusions 110 and 1101 thereof couple to and extend-angularly/radially outwards of the connector portion thereof. Referring to FIG. 10, corresponding radially inward-facing and circumferentially spaced-apart recessed portions 102A and 102B of outer sleeve 94 enable movement of elongate protrusions 110 and 1101 axially inwards as seen by arrows 103A and 103B in FIG. 9 towards and so as to extend more parallel with corresponding connector portion 1102. Elongate protrusions 110 and 1101 so fully deflected/biased inwards towards connector portion 1102 as seen in FIG. 9, have laterally-extending minimum distance D_{MIN} adjacent distal ends thereof.

Referring back to FIG. 10, for each male member 104A, protrusion 110 and connector portion 1102 and protrusion 1101 and connector portion 1102 thereof may be considered or comprise a pair of respective hooks or catches, with only one pair thereof or one said hook or catch being provided in other embodiments. Elongate protrusions 110 and 1101 and connector portion 1102 in this non-limiting example couple together so as to form an arrow shape which points towards applicator 34 (and/or distal end 38 thereof).

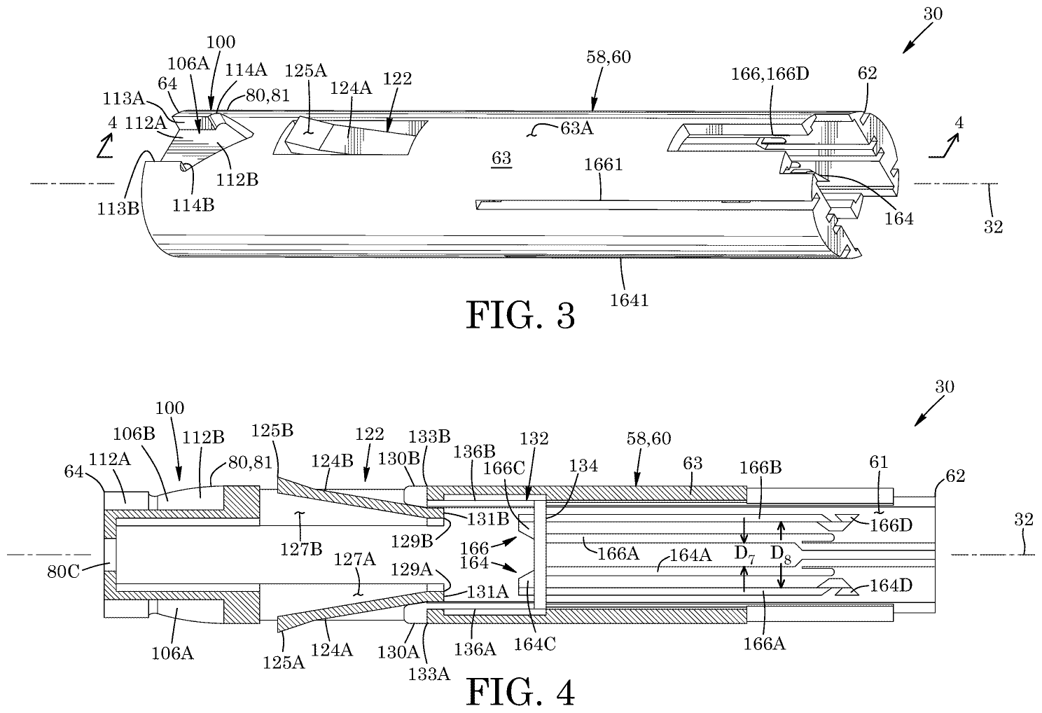

As seen in FIG. 3, each female member 106A of cap locking mechanism 100 comprises one or more recessed portions: in this non-limiting example a first recessed portion 112A and a second recessed portion 112B in fluid communication with the first recessed portion thereof. For each female member the first recessed portion thereof extends from distal end 64 towards proximal end 62 of pod 58. As seen in FIG. 10, each first recessed portion 112A is elongate shaped to facilitate the passage of a corresponding male member therethrough. In this example each first recessed portion in this non-limiting embodiment comprises axially-extending and/or smooth opposed walls 113A and 113B in this non-limiting example and/or is rectangular in side profile. Referring to FIG. 10, each first recessed portion 112A spans a laterally-extending distance D_{R} which is less than the laterally-extending maximum distance D_{MAX} of elongate protrusions 110 and 1101 of male member 104A.

For each female member 106A each second recessed portion 112B is triangular in side profile in this non-limiting embodiment. Each female member is thus triangular at least in part in this non-limiting example. As seen in FIG. 3, each female member 106A includes in this non-limiting embodiment at least one and in this example a pair of opposed or mirrored catches 114A and 114B. Each catch is a triangular and/or a triangular prism in shape in this non-limiting embodiment. As seen in FIG. 10, each pair of catches 114A and 114B is laterally spaced a distance D_{F} which generally or substantially equals to the laterally-extending maximum distance D_{MAX} of spanning distal ends of elongate protrusions 110 and 1101 of male member 104A.

As seen with reference to FIGS. 3 and 6, pod cap assembly 82 couples to pod 58 via cap locking mechanism 100, in this non-limiting example snap-fitting in place until the pod is inserted into applicator 34 seen in FIG. 9. In order to couple pod cap assembly 82 to pod 58, inner sheath 90 seen in FIG. 7 aligns with and is positioned to receive needle 76 therewithin. During this process each male member 104A seen in FIG. 10 is moved axially towards and aligns with respect to first recessed portions 112A of a corresponding female member 106A. This causes elongate protrusions 110 and 1101 to deflect radially inwards towards connector portion 1102, which in turn enables the male members to continue to pass through the first recessed portions to second recessed portions 112B of the female members as seen in FIG. 6. Each second recessed portion is shaped to couple with and/or snugly receive male member 104A including elongate protrusions 110 and 1101. Each female member 106A includes at least one and in this example a pair of spaced-apart catches 114A and 114B in fluid communication with second recessed portion 112B thereof. The catches are shaped to engage elongate protrusions 110 and 1101 thereafter and inhibit dislodgement of the elongate protrusions therefrom, thus functioning to lock pod cap assembly 82 to forward end portion 80 of pod 58.

As seen in FIG. 10, cap locking mechanism 100 may be unlocked via one or more members that facilitate moving or actuating of the cap locking mechanism from the locked mode seen in in FIG. 6, to an unlocked position seen in FIG. 9. The following is a non-limiting embodiment which achieves this functionality. As seen in FIG. 7, applicator 34 includes at least one actuating member, in this non-limiting embodiment in the form of at least one and in this non-limiting example a pair of circumferentially spaced-apart deflecting members 116A and 116B. Referring to FIG. 9, each deflecting member is shaped to dislodge corresponding male member 104A from its female member 106A upon pod 58 being inserted into and/or coupling to applicator 34. Each male member is moveable from its locked position seen in FIG. 6 to its unlocked position seen in FIG. 9 via its corresponding deflecting member. The following are additional non-limiting particulars which achieve this functionality.

As seen in FIG. 10, each deflecting member 116A is positioned adjacent distal end 38 of applicator 34 in this example. Each deflecting member extends from the distal end of the applicator towards proximal end 36 of the applicator seen in FIG. 8 in this non-limiting embodiment. Referring back to FIG. 10, each deflecting member 116A is longitudinally extending in this non-limiting embodiment. Each deflecting member is C-shaped in side profile in this non-limiting example. Each deflecting member 116A includes a recessed portion 118A of applicator 34 in this non-limiting embodiment. Each recessed portion is slot-shaped in this non-limiting example. For each deflecting member 116A, recessed portion 118A thereof has a laterally-extending slot width W_{S} which is less than distance D_{C} between catches 114A and 114B. Recessed portions 118A may be referred to as first recessed portions of applicator 34.

As seen in FIG. 8, pod cap assembly 82 so locked in place via cap locking mechanism 100, is configured to inhibit positioning of forward end portion 80 of pod 58 in the injection mode seen in FIG. 13 when the pod cap assembly is coupled to the pod. Retraction of the pod (as shown by arrow 119 in FIG. 8) into applicator 34 is thus inhibited when the pod cap assembly is coupled to the pod as seen in FIG. 8.

To remove pod cap assembly 82 and referring to FIG. 5, pod 58 is inserted axially (as shown by arrow 120) into receptacle 52 of applicator 34 via distal end 38 of the applicator. As seen in FIG. 8, outer sleeve 94 of pod cap assembly 82 is moved axially so as to abut and/or receive distal end 38 of applicator 34, with male members 104A seen in FIG. 9 being positioned to align axially with deflecting members 116A. Pod 58 continues to be inserted into the applicator until distal portions of male members 104A extend within and abut recessed portions 118A of deflecting members 116A. This causes elongate protrusions 110 and 1101 of the male members to dislodge from catches 114A and 114B, with the elongate protrusions thus moving from a locked position seen in FIG. 6, to an unlocked position seen in FIG. 9. Insertion of pod 58 into applicator 34 thus causes male members 104A to dislodge from female members 106A at least in part.

Still referring to FIG. 9, cap locking mechanism 100 in its unlocked position enables pod cap assembly 82 to be manually movable axially outwards (as shown by arrow 102C in FIG. 9) relative to pod 58 so as to remove the pod cap assembly from the pod thereafter according to one non-limiting embodiment. This may be achieved by pulling gripping member 88 axially outwards as seen with reference to FIGS. 9 and 10.

In addition or alternatively, cap locking mechanism 100 may be configured such that insertion of pod 58 into applicator 34 and/or coupling of the pod with the applicator, causes pod cap assembly 82 to be automatically be released from the pod. For example, in one non-limiting embodiment the movement/bending inwards of elongate protrusions 110 and 1101 towards connector portion 1102 seen in FIG. 9 due to deflecting member 116A, may result in a spring bias which promotes movement of the pod cap assembly axially outwards from the pod and applicator in response thereto.

Pod cap assembly 82 as herein described is thus configured to enclose distal end 64 of pod 58 until pod 58 is inserted within and/or couples to receptacle 52. The pod cap assembly is therefore configured to render medicament 68 inaccessible when the pod is free/spaced from, not inserted into and/or not coupled to applicator 34.

The interaction between and selective coupling together of applicator 34 and pod 58 according to one non-limiting embodiment will now be discussed in more detail. Referring to FIG. 8, medicament injector 30 includes a first coupling mechanism 122 which couples together the applicator and pod. The first coupling mechanism is configured to inhibit axial movement of pod 58 relative to applicator 34 in the locked protected needle pre-injection mode thereof. The following is a non-limiting embodiment which achieves this functionality.

First coupling mechanism 122 includes a first of male and female member(s), in this non-limiting embodiment at least one and in this example a pair of circumferentially spaced-apart latch members, in this case arms or applicator-connecting arms 124A and 124B. Each applicator-connecting arm is elongate and resilient. Applicator-connecting arms 124A and 124B extend longitudinally at least in part and laterally in part in this non-limiting example. Each applicator-connecting arm is configured to bias radially outwards at least in part. Pod 58 includes applicator-connecting arms 124A and 124B of first coupling mechanism 122 as parts thereof in this non-limiting example. The applicator-connecting arms of the first coupling mechanism may be referred to as first latch members of pod 58 and/or a pair of prongs. Applicator-connecting arms 124A and 124B are positioned near and/or closer to and face at least in part distal end 64 of pod 58 in this non-limiting example. The applicator-connecting arms may thus be said to be located closer to the patient side or end 64 of pod 58. As seen in FIG. 7, applicator-connecting arms 124A and 124B include distal end portions 125A and 125B biased to be positioned radially outwards from pod 58. The distal end portions of the applicator-connecting arms of first coupling mechanism 122 protrude at least in part and/or are outwardly flared in this non-limiting example; however, this is not strictly required.

As seen in FIG. 8, first coupling mechanism 122 includes a second of male and female member(s), in this non-limiting embodiment at least one and in this example a pair of circumferentially spaced-apart recessed portions 126A and 126B. As seen with reference to FIGS. 6 and 9, for the first coupling mechanism each recessed portion 126A thereof is shaped to at least partially receive a respective applicator-connecting arm 124A thereof, in this non-limiting example distal end portions 125A of the applicator-connecting arms. Referring back to FIG. 8, applicator 34 comprises recessed portions 126A and 126B of first coupling mechanism 122 in this non-limiting embodiment. However, this is not strictly required: applicator 34 may comprise applicator-connecting arms 124A and 124B and pod 58 may comprise corresponding the recessed portions in other embodiments. Recessed portions 126A and 126B may be referred to as second recessed portions of applicator 34. For first coupling mechanism 122, the recessed portions thereof in this non-limiting embodiment comprise apertures extending through wall 42 of the applicator. Recessed portions 126A and 126B are positioned near, closer to and/or adjacent distal end 38 of applicator 34 in this non-limiting example. The recessed portions of first coupling mechanism 122 are rectangular in outer/side shape in this non-limiting embodiment.

In operation and referring to FIG. 2, applicator-connecting arms 124A and 124B of first coupling mechanism 122 are configured to extend radially outwards at least in part (in this example distal end portions 125A and 125B thereof) relative to pod housing 60 when pod 58 is spaced/free from applicator 34. Referring to FIGS. 6 and 9, the pod is inserted into the applicator such that the applicator-connecting arms align with respective recessed portions 126A. Still referring to FIGS. 6 and 9, applicator-connecting arms 124A extend into receptacle 52 of applicator 34, pass through/by deflecting members 116A, are deflected radially inwards by wall 42 of the applicator towards axis 32 and thereafter extend radially outwards once more at least in part so that distal end portions 125A of the applicator-connecting arms extend into/through recessed portions 126A. As seen in FIG. 7, the distal end portions of applicator-connecting arms 124A and 124B of first coupling mechanism 122 are configured to extend radially outwards at least in part relative to pod housing 60 when the applicator-connecting arms are so received within the recessed portions of the first coupling mechanism. Pod 58 may thus be said to be coupled to or snap-fit connected to applicator 34.

Referring to FIG. 8, first coupling mechanism 122 in its coupled position is configured to inhibit axially/distally outwards movement of pod 58 relative to applicator 34 thereafter in direction shown by arrow 128, until the first coupling mechanism is removed/dislodged from its coupled position. In this non-limiting example, distal end portions 125A and 125B of applicator-connecting arms 124A and 124B are configured to abut laterally-extending portions 42A and 42B of wall 42 of applicator 34 adjacent recessed portions 126A and 126B, with said laterally-extending portions functioning as stops to inhibit axially/distally outwards movement of the pod relative to the applicator. The applicator-connecting arms are thus configured to promote locking/holding in place of pod 58 upon and subsequent to the pod being loaded into the applicator.

As seen in FIG. 4, pod housing 60 has a pair of longitudinally-extending slots 127A and 127B within which applicator-connecting arms 124A and 124B extend and are moveable relative thereto in this non-limiting embodiment. The slots axially align with and are axially spaced rearward of female members 106A and 106B of cap locking mechanism 100 in this non-limiting example. Applicator-connecting arms 124A and 124B couple to and are integrally formed with pod 58 in this non-limiting embodiment; however, this is not strictly required. The applicator-connecting arms have proximal end portions 129A and 129B that are radially inwardly spaced from distal end portions 125A and 125B thereof in this example. The proximal end portions of applicator-connecting arms 124A and 124B are radially inwardly positioned from wall 63 of pod housing 60 so as to form longitudinally-extending slots 131A and 131B therebetween in this non-limiting example.

To decouple first coupling mechanism 122 so as to enable movement of pod 58 relative to applicator 34, pod cap assembly 82 is first removed as seen in FIG. 11 and described above. Distal end 64 of the pod is retracted from its pre-injection mode to an injection mode: moving axially rearward as seen by arrow 83 in FIG. 13 upon pressing against or abutting skin 85A of person 85 so as to overcome outwardly biasing force of spring 78. This causes distal end portions 125A of applicator-connecting arms 124A of first coupling mechanism 122 to abut first actuating members, in this example deflecting members, in this example portions 135A and 135B of applicator 34 adjacent recessed portions 126A. This causes the applicator-connecting arms of the first coupling mechanism to bias radially inwards (as seen by arrows 137A and 137B in FIG. 13) towards axis 32 during the injection mode. Medicament injector 30 so configured is thus moveable from its pre-injection mode seen in FIG. 11 to its injection mode seen in FIGS. 13 and 14.

As seen in FIG. 4, first coupling mechanism 122 in this non-limiting embodiment includes at least a second actuating member, in this non-limiting example in the form of at least one additional and in this non-limiting case a pair of circumferentially spaced deflecting members 130A and 130B. The deflecting members are configured to inhibit re-coupling of pod 58 within applicator 34 after injection has occurred in the first instance. The following is a non-limiting embodiment which achieves this functionality.

Deflecting members 130A and 130B are a part of a deflector assembly 132 configured to fit within and be slidable/axially-movable relative to pod housing 60. The pod 58 comprises the deflector assembly in this non-limiting example. As seen in FIG. 4, deflecting members 130A and 130B are shaped to extend along outer or exterior sides of applicator-connecting arms 124A and 124B of first coupling mechanism 122. The deflecting members are generally rectangular prisms in shape in this non-limiting example; however, here too this is not strictly required. Deflecting members 130A and 130B are outwardly convex in a distal-facing direction or in a direction facing distal end 64 of pod 58; however, the latter is not strictly required.

Deflector assembly 132 has a first or retracted position seen in FIG. 4. Deflecting members 130A and 130B are shaped to inhibit retraction of the deflector assembly axially rearward of the retracted position. To achieve this functionality in this non-limiting example deflecting members 130A and 130B are positioned adjacent slots 131A and 131B, respectively, so as to abut laterally-extending portions, ledges or shoulders 133A and 133B of pod housing 60 in the retracted position of deflector assembly 132. The shoulders are in fluid communication with the slots.

Deflector assembly 132 is configured to couple together deflecting members 130A and 130B thereof so as to move as one, in this non-limiting example via a hub 134 thereof. The hub is annular in this non-limiting embodiment and shaped to snugly fit within interior 61 of and be slidable relative to pod 58. As seen in FIG. 11, the deflector assembly including the hub thereof, extends about cartridge 65, in this case barrel 66 of syringe assembly 70 in this non-limiting embodiment.

Referring back to FIG. 4, deflector assembly 132 includes at least one and in this example a pair of circumferentially spaced-apart and elongate connectors 136A and 136B via which deflecting members 130A and 130B couple to hub 134 in this non-limiting embodiment. As seen in FIG. 4, the connectors extend substantially parallel to and are radially offset from axis 32 in this non-limiting example. Connectors 136A and 136B are shaped to extend between respective applicator-connecting arms 124A and 124B and wall 63 of pod housing 60: in this non-limiting example the connectors slidably extend through respective slots 131A and 131B. Deflecting members 130A and 130B are enlarged relative to connectors 136A and 136B and slots 131A and 131B, thus inhibiting the deflecting members moving axially rearward through/past the slots. Connector 136A with deflecting member 130A coupled thereto and connector 136B with deflecting member 130B coupled thereto, may be referred to collectively as a pair of arms.

Deflector assembly 132 is moveable from its first or retracted position seen in FIG. 4, to a second or extended position seen in FIGS. 14 to 17. The deflector assembly operatively abuts cartridge 65 (and/or barrel 66 or syringe assembly 70), with forward movement of the cartridge (and/or barrel or syringe assembly) axially towards distal end 64 of pod 58 functioning to move the deflector assembly forward to the second, extended position. When medicament injector 30 is ready to be used and referring to FIG. 13, forward end portion 80 of pod 58 is retracted as shown by arrow 83. This causes deflector assembly 132 to be biased/pushed axially forward relative to the pod, which causes deflecting members 130A and 130B of the deflector assembly to push down or bias radially inwards applicator-connecting arms 124A and 124B of first coupling mechanism 122. This thereby causes the applicator-connecting arms of the first coupling mechanism to release from recessed portions 126A and 126B or unlock pod 58 from applicator 34. The pod is thus moved from the locked protected needle pre-injection mode of FIG. 8 to an unlocked protected needle pre-injection mode seen in FIG. 11.

Still referring to FIG. 11, medicament injector 30 in this non-limiting example includes a second coupling mechanism 138. The second coupling mechanism is axially spaced-apart from first coupling mechanism 122 in this non-limiting example. Second coupling mechanism 138 is configured to i) inhibit axial movement of syringe assembly 70 (or cartridge 65, barrel 66 and/or needle 76 thereof) relative to pod 58 in the unlocked protected needle pre-injection mode seen in FIG. 11 and ii) enable selective release of plunger 54 and in this non-limiting example syringe assembly 70 axially forward towards distal end 64 of pod 58 in the injection mode seen in FIG. 14. The following is a non-limiting embodiment which achieves this functionality.

As seen in FIG. 8, second coupling mechanism 138 includes a first of male and female member(s), in this non-limiting embodiment at least one and in this example a pair of circumferentially spaced-apart latch members or arms, in this case cartridge-retaining arms 140A and 140B. Each cartridge-retaining arm herein is elongate and resilient. Pod 58 comprises cartridge-retaining arms 140A and 140B of second coupling mechanism 138 in this non-limiting example. The cartridge-retaining arms of the second coupling mechanism may be referred to as second latch members of the pod. For pod 58 cartridge-retaining arms 140A and 140B thereof are axially spaced rearwards of applicator-connecting arms 124A and 124B thereof. Cartridge-retaining arms 140A and 140B include proximal end portions 142A and 142B operatively connected to pod housing 60 and distal end portions 144A and 144B axially spaced-apart from the proximal end portions thereof. The proximal end portions of the cartridge-retaining arms are adjacent and/or couple to insert 67 of pod 58. The insert of the pod is annular in this non-limiting example and integrally couples to arms 124A and 124B so as to form a unity whole. Cartridge-retaining arms 140A and 140B of second coupling mechanism 138 may thus be said to be located adjacent the butt end of pod 58 in this non-limiting example.

Distal end portions 144A and 144B of cartridge-retaining arms 140A and 140B are enlarged in this non-limiting example. As seen in FIG. 2, cartridge-retaining arms 140A and 140B (including the distal end portions thereof) of second coupling mechanism 138 are configured to bias radially outwards from pod housing 60 prior to being inserted into applicator 34.

As seen in FIG. 17, the second coupling mechanism includes a second of male and female member(s), in this non-limiting embodiment at least one and in this example a pair of circumferentially spaced-apart recessed portions 146A and 146B. Applicator 34 comprises the recessed portions of second coupling mechanism 138 in this non-limiting embodiment. However, this is not strictly required and the applicator may comprise cartridge-retaining arms 140A and 140B and pod 58 may comprise recessed portions 146A and 146B in other embodiments. Recessed portions 146A and 146B may be referred to as third recessed portions of applicator 34. For the applicator, recessed portions 146A and 146B thereof are axially spaced rearwards of recessed portions 126A and 126B thereof in this example. The recessed portions of second coupling mechanism 138 extend within proximal portion 48 of outer body 40 of applicator 34 in this non-limiting example.

As seen in FIG. 13, recessed portions 146A and 146B of second coupling mechanism 138 are shaped to selectively at least partially receive respective cartridge-retaining arms 140A and 140B thereof, in this non-limiting example distal end portions 144A and 144B of the cartridge-retaining arms. Recessed portions 146A and 146B are shaped to mate and snugly receive the cartridge-retaining arms at least in part in this non-limiting example. The following is a non-limiting embodiment which achieves this functionality.

As seen in FIG. 13, recessed portions 146A and 146B of second coupling mechanism 138 extend from inner surface 44 towards outer surface 46 of wall 42 of applicator 34 in this non-limiting embodiment. The recessed portions have first shapes and distal end portions 144A and 144B of cartridge-retaining arms 140A and 140B have second shapes configured to mirror the first shapes so as to facilitate matting therewith in this non-limiting embodiment. In this non-limiting example recessed portions 146A and 146B are curved in a radially inward facing direction, in this example outwardly concave along inner surfaces thereof 148A and 148B. Distal end portions 144A and 144B are curved in a radially outward facing direction, in this example being outwardly convex along radially outward surfaces 150A and 150B thereof.

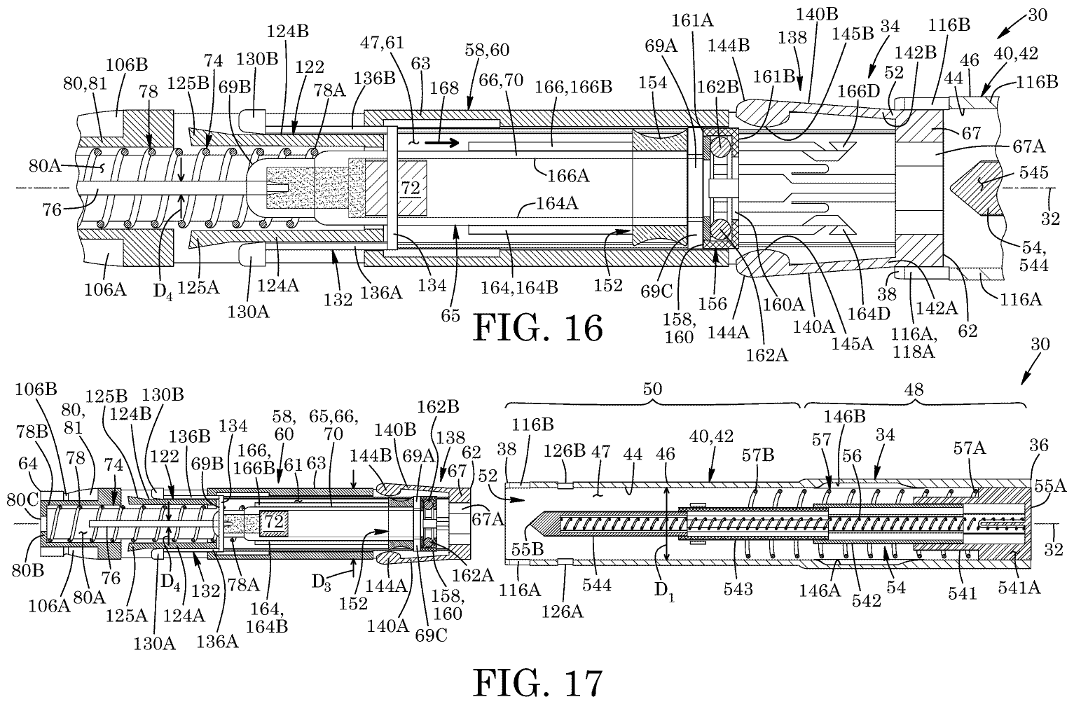

As seen in FIG. 16, second coupling mechanism 138 in this non-limiting embodiment includes an additional coupling member, in this example a sleeve 152 shaped to extend about syringe assembly 70, in this example barrel 66 thereof. The sleeve is positioned to abut a forward/distal end of flange 69C of the cartridge/barrel and be positioned adjacent proximal end 69A of the barrel in this non-limiting example. As seen in FIG. 5, the sleeve is positioned to align with distal end portions 144A and 144B of cartridge-retaining arms 140A and 140B prior and during insertion of pod 58 into applicator 34. The applicator is shaped to bias the cartridge-retaining arms radially inwards during the insertion process, with sleeve 152 being shaped/aligned to facilitate/enable the cartridge-retaining arms to so bias radially inwards during the locked protected needle pre-injection mode of medicament injector 30 seen in FIG. 8. Cartridge-retaining arms 140A and 140B so radially inwardly biased function to compress and/or transmit a radially inwardly extending force to sleeve 152, which in turn abuts, frictionally engages with and/or couples to cartridge 65 (or barrel 66 thereof) via an interference fit. The cartridge-retaining arms so biased against the sleeve to squeeze about the cartridge/barrel, functions to inhibit movement of syringe assembly 70 (and/or cartridge 65 or barrel 66 thereof) relative to pod housing 60. Sleeve 152 may thus remain adjacently aligned with distal end portions 144A and 144B of cartridge-retaining arms 140A and 140B. This may ensure that the sleeve remains clamped in place during the unlocked portion of pre-injection mode seen in FIG. 8 where the pod cap assembly is removed. Sheath 90 of pod cap assembly 82 also inhibits and/or prevents axial or longitudinal movement of cartridge 65 (and/or syringe and needle assembly 70) during insertion of pod 58 into applicator 34 seen in FIG. 5.

Thus in operation and referring to FIG. 2, insertion of pod 58 into applicator 34 causes wall 42 of the applicator to bias distal end portions 144A and 144B of cartridge-retaining arms 140A radially-inwards. The cartridge-retaining arms of second coupling mechanism 138 are thus configured to at least initially be held down or biased to radially inward positions by the applicator body's inner walls when the pod is inserted therein. As seen in FIG. 8, distal end portions 144A and 144B of cartridge-retaining arms 140A and 140B so biased radially-inwards, align with and compress sleeve 152 to hold the syringe assembly 70 (and/or cartridge 65 or barrel 66 thereof) in place in the locked protected needle pre-injection mode. Medicament injector 30 (including second coupling mechanism 138 thereof) is configured such that the force with which the syringe assembly is held in place is greater than and/or sufficient to overcome the axially outwardly biasing forces from inner coil spring 56. Sleeve 152 is thus configured to inhibit inadvertent/unwanted/premature forward movement syringe assembly 70 which may otherwise inadvertently cause needle 76 to deploy out of pod 58.

Distal end portions 144A and 144B of cartridge-retaining arms 140A and 140B have inner surfaces 145A and 145B that are shaped in this non-limiting embodiment to mirror the shape of outer surface 154 of the sleeve. Sleeve 152 has an outer surface in this non-limiting embodiment which is curved, in this example being annular and outwardly concave in lateral section and/or in radially outward-facing directions. Inner surfaces 145A and 145B of cartridge-retaining arms 140A and 140B are in this non-limiting embodiment curve, in this example outwardly convex in radially inward-facing directions.

Pod 58 continues to be inserted into applicator 34 until first coupling mechanism 122 causes the pod to snap-fit connect with the applicator via recessed portions 126A and 126B. Referring to FIG. 11, medicament injector 30 is configured such that when the pod is so coupled to the applicator via the first coupling mechanism, cartridge-retaining arms 140A and 140B of second coupling mechanism 138 are axially forward of recessed portions 146A and 146B. This ensures that movement of syringe assembly 70 (and/or cartridge 65 or barrel 66 thereof) relative to pod housing 60 continues to be inhibited in the unlocked protected needle pre-injection mode.

As seen with reference to FIGS. 5 and 8, loading of pod 58 into applicator 34 functions to prime the drive element, in this example plunger 54, with the plunger moving from its spring-biased extended position seen in FIG. 5 to its primed or retracted position seen in FIG. 8. Telescoping members 541, 542, 543, and 544 are thus configured to be compressed to a primed or retracted position seen in FIG. 8 by the action of inserting the pod into the applicator. Medicament injector 20 is thus configured such that the force required to manually insert pod 58 into applicator 34 (that is the force to overcome the spring forces of springs 56 and 57), primes plunger 54, with potential energy being stored by springs 56 and 57 so compressed as a result thereof.

In order to perform an injection and referring to FIG. 13, medicament injector 30 is configured such that pushing distal end 64 of pod 58 against skin 85A of person 85 or retraction of pod 58 into applicator 34 as seen by arrow 83, releases stored potential energy thereof (in this example from springs 56 and 57) and causes the drive element in this case plunger 54, to engage with the pod so as to output medicament 68 as seen in FIG. 15. Thus, retraction of the pod into the applicator as seen in FIG. 13 actuates/releases/fires the plunger so primed to further engage with the pod and/or cartridge 65 and/or syringe assembly 70 so as to output the medicament seen in FIG. 15. Medicament injector 30 is thus configured such that i) insertion or retraction of pod 58 into applicator 34 in the first instance functions to couple the pod to the applicator as seen with reference to FIGS. 5 and 11 and ii) further retraction of the pod into the applicator (e.g. in the second instance) releases the drive element or plunger 54 in this non-limiting embodiment to engage the pod so as to output a medicament automatically therefrom. Medicament injector 30 may thus be referred to as and/or comprise an autoinjector. In this non-limiting example at least front end portion 80 of the pod or pod housing 60 thereof is spring biased outwards from applicator 34 and configured selectively retract at least in part within the applicator to actuate release of the medicament.

The following are additional non-limiting details which achieve the above functionality. Referring to FIG. 8, abutting distal end 64 of the pod against skin 85A of person 85 in this non-limiting example causes forward end portion 80 of pod housing 60 to move from the pre-injection mode to initiate the injection mode in FIG. 13, axially rearward as seen by arrow 83, compressing spring 78 to expose needle 76 in the injection mode of medicament injector 30 seen in FIGS. 14 and 15. This movement axially rearwardly causes cartridge-retaining arms 140A and 140B of second coupling mechanism 138 seen in FIG. 14 to align with recessed portions 146A and 146B, thereby enabling cartridge-retaining arms 140A and 140B to move/bias radially outwards and at least partially therein during the injection mode of medicament injector 30. The cartridge-retaining arms so radially outwardly positioned, no longer compress/bias against sleeve 152 to the same if any extent, thus removing and/or reducing the extent to syringe assembly 70 (and/or cartridge 65 or barrel 66 thereof) is held/clamped/squeezed in place via sleeve 152. In one non-limiting embodiment, distal end portions 144A and 144B of cartridge-retaining arms 140A and 140B are configured to be radially outwardly spaced from the sleeve when the cartridge-retaining arms are positioned within recessed portions 146A and 146B of second coupling mechanism 138, thereby reducing and/or removing any compression force. This in turn enables plunger 54 to move from its retracted position seen in FIG. 13 to a partially extended position as seen in FIG. 14. This is in turn causes cartridge 65 and barrel 66 of syringe assembly 70 to move axially forward towards distal end 64 of pod 58, with needle 76 extending outwards and into skin 85A of person 85. For second coupling mechanism 138, cartridge-retaining arms 140A and 140B thereof are thus configured to fit at least in part within recessed portions 146A and 146B thereof so as to enable axial movement of the syringe assembly in the injection mode.

Wall 42 of outer body 40 of applicator 34 is thus configured to keep cartridge-retaining arms 140A and 140B in place until a certain point. When the tip or end portion 80 of medicament injector 30 so assembled is pressed into person 85, the effective inner diameter of the body of the applicator may thus be said to widen (in this example via recessed portions 146A and 146B), enabling the cartridge-retaining arms to spring back/radially-outwards to or towards their original positions when pod 58 was not inserted within applicator 34. This act releases syringe assembly 70 (and/or cartridge 65, barrel 66 and needle 76 thereof) and allows plunger 54 to push the syringe assembly forward, and push the needle into the person.

Referring to FIG. 14, the release of plunger 54 and inner coil spring 56 causes deflector assembly 132 to be biased/pushed axially forward along with syringe assembly 70 (and/or cartridge 65 or barrel 66 thereof). As seen in FIG. 16, deflector assembly 132 in its extended position functions to further deflect/bias/move applicator-connecting arms 124A and 124B radially inwards so as to inhibit their extending radially outwards once more. Deflecting members 130A and 130B are positioned closer to distal end portions 125A and 125B of the applicator-connecting arms when the deflector assembly is its extended position. Deflector assembly 132 so positioned is shaped/configured to inhibit further retraction thereof anew: this may be due to frictional interference between i) shoulders 133A and 133B and connectors 136A and 136B and ii) applicator-connecting arms 124A and 124B in one non-limiting embodiment. The deflector assembly so positioned inhibits subsequent movement radially outwards of the applicator-connecting arms.

Still referring to FIG. 16, medicament injector 30 in this non-limiting embodiment further includes a safeguard mechanism 156 coupled to pod 58. Second coupling mechanism 138 (and/or cartridge-retaining arms 140A and 140B and/or sleeve 152 thereof) may be referred to as parts of the safeguard mechanism. Safeguard mechanism 156 may be referred to as a coupling mechanism and/or a guiding mechanism configured to i) enable cartridge (and/or syringe and needle assembly thereof) to move axially during the injection mode as seen in FIGS. 14 and 15, ii) temporarily lock in place axially forward distally from the initial position in the injection mode of FIG. 14, and iii) permanently lock in place near/adjacent proximal end 62 of pod 58 as seen in FIG. 17 upon the pod being removed from applicator 34 thereafter. The safeguard mechanism is thus configured, upon the pod being removed from applicator 34 after use thereof, to inhibit reinsertion of the pod into the applicator once more. The safeguard mechanism is additionally configured in this non-limiting example to inhibit access to and/or pushing on a rear end 69A of syringe assembly 70 (and/or cartridge 65 or barrel 66 thereof). As seen in FIG. 15, safeguard mechanism 156 is additionally configured in this non-limiting example to inhibit spring 78 from pushing cartridge 65 (and/or syringe and needle assembly thereof) in an axially rearward direction when the needle 76 is fully extended and medicament 68 is in the process of being delivered by the outward bias of plunger 54. The following is a non-limiting embodiment which achieves this functionality.

Referring to FIG. 16, safeguard mechanism 156 includes a second or rearward barrier member 158. The rearward barrier member may be referred to as a syringe barrier member. Rearward barrier member 158 is configured to inhibit plunger 54 from passing therethrough to cartridge 65 in the unlocked protected needle pre-injection mode of medicament injector 30 as seen in FIG. 11. The rearward barrier member is configured to enable the plunger to pass thereby and/or therethrough in the injection mode as seen in FIG. 15. Plunger 54 is thus configured to extend through safeguard mechanism 156 during the injection mode in this non-limiting embodiment. Referring to FIG. 17, rearward barrier member 158 is configured to inhibit passage of plunger 54 therepast after cartridge 58 has been removed from applicator 34. The rearward barrier member is configured to inhibit re-insertion of the pod 58 in the applicator 34 by preventing the inward radial movement of the latch members 140A and 140B after pod 58 has been removed from applicator 34. The following are additional non-limiting particulars of an embodiment which achieves this functionality.

As seen in FIG. 5, rearward barrier member 158 has a first or retracted mode or position adjacent the proximal end portion of pod 58, in this example being towards proximal end 62 of the pod. Referring to FIG. 8, the rearward barrier member is biased towards distal end 64 of the pod in this non-limiting embodiment, in this example via spring 56 of plunger 54 causing telescoping member 544 to abut thereon. Rearward barrier member 158 is configured to be moveable axially towards distal end 64 of pod 58 during the injection mode seen in FIG. 15. The forward positioning of sleeve 152 enables cartridge-retraining arms 140A and 140B to be pushed forward via spring 57 and bias radially inwards anew so as to be become axially forward of at least in part and dislodged from recessed portions 146A and 146 of pod 58. Upon removal of pod 58 from applicator 34 thereafter as seen in FIG. 17, the rearward barrier member is biased (in this example via spring 78) to move axially towards proximal end 62 of pod 58 anew. Rearward barrier member 158 is configured, upon the pod being removed from applicator, to inhibit movement thereof towards distal end 64 of the pod thereafter. Yet further non-limiting particulars which achieve this functionality are set out below.

As seen in FIG. 16, rearward barrier member 158 in this non-limiting example includes a body or housing 160. The housing is annular in this example and has a bore 160A extending therethrough. As seen in FIG. 15, the bore is sized to inhibit member 543 of plunger 54 from passing therethrough and enable member 544 of the plunger to pass therethrough; however, this is not strictly required nor necessary for medicament injector 30 to function in the manner described herein.

Referring back to FIG. 16, rearward barrier member 158 in this non-limiting embodiment comprises at least one and in this example a pair of spaced-apart protrusions or pins 162A and 162B positioned therewithin. Housing 160 is shaped to receive the pins therewithin and at least partially enclose the pins in this example via a pair of annual, radially-extending forward and rearward flanges 161A and 161B. Telescoping member 543 of plunger 54 in this non-limiting example is shaped to selectively abut the rearward flange 161B of housing 160 as seen in FIG. 15; however, here too this is not strictly required nor necessary for medicament injector 30 to function in the manner as herein described. Forward flange 161A of housing 160 is shaped/positioned to abut a rearward/proximal end of flange 69C of barrel 66 of syringe assembly 70 in this example and be positioned adjacent proximal end 69A of the barrel in this non-limiting example. The housing is shaped to enable radial movement of pins 162A and 162B relative to the housing.

Referring to FIG. 3, pod 58 includes at least one and in this example two pairs of circumferentially spaced-apart passageways 164 and 1641 and 166 and 1661 via which the pins of rearward barrier member 158 extend between and are moveable therealong, respectively. Passageways 1641 and 1661 are on opposite sides of pod housing 60 compared to passageways 164 and 166 and are substantially the same as passageways 164 and 166. Accordingly, only passageways 164 and 166 will be described in detail and these are best seen in FIG. 4. The passageways are positioned within interior 61 of pod 58 and are formed along and/or with pod housing 60 in this non-limiting example. Passageways 164 and 166 are elongate, are axially spaced from each other and substantially extend parallel to axis 32 of medicament injector 30 in this non-limiting embodiment.

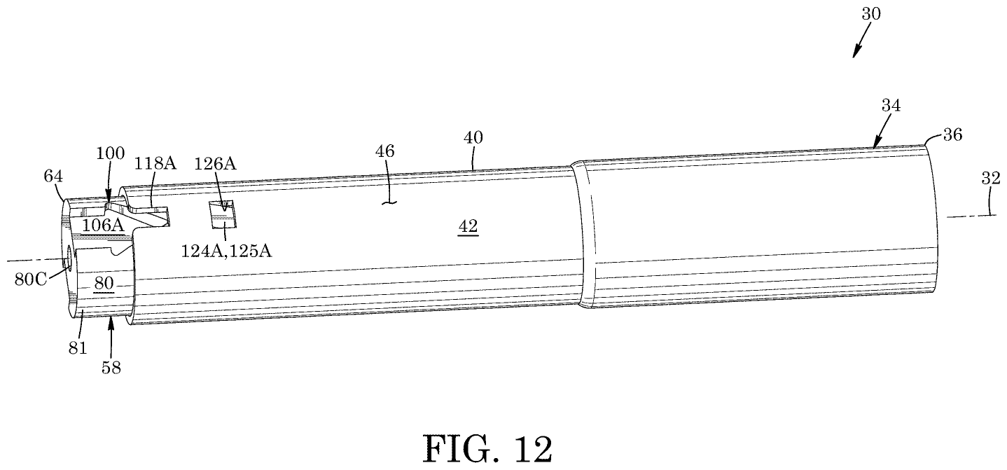

As seen in FIG. 4, the passageways include inner portions 164A and 166A that are elongate and axially spaced from each other. The inner portions of passageways 164 and 166 may be referred to as tracks or inner tracks. Inner portions 164A and 166A of the passageways are spaced-apart laterally/radially by a distance D₇. The inner portions of passageways 164 and 166 so spaced apart are configured to position pins 162A and 162B to inhibit passage of distal end portion 545 of plunger 54 therethrough as seen in FIGS. 8 and 11 to 14. Rearward barrier member 158 including the pins thereof so positioned within housing 160 thereof, is configured to keep/inhibit the plunger from prematurely pushing on/against piston 72. In this non-limiting embodiment, pins 162A and 162B follow tracks or inner portions 164A and 166A of passageways 164 and 166 seen in FIG. 4 in wall 63 of pod housing 60 which keeps the space/opening or distance D₇ between the pins small enough so that plunger 54 seen in FIGS. 8 and 11 to 14 cannot go past and/or therethrough. The pins are thus spaced via inner portions 164A and 166A of passageways 164 and 166 seen in FIG. 4 to inhibit passage of plunger 54 therethrough. Referring to FIGS. 13 and 14, pins 162A and 162B are moveable axially via the passageways towards distal end 64 of pod 58 in the injection mode to enable passage of plunger 54 to move syringe assembly 70 (and/or cartridge 65, barrel 66 and needle 76 thereof) forward.

Referring back to FIG. 4, passageways 164 and 166 in this example include outer portions 164B and 166B. The outer portions of the passageways may be referred to as tracks or outer tracks. Outer portions 164B and 166B of the passageways extend substantially parallel to and are radially outwardly spaced relative to inner portions 164A and 166A of the passageways in this non-limiting example. The inner and outer portions of passageways 164 and 166 extend from near adjacent proximal end 62 of pod 58 towards distal end 64 of the pod. Outer portions 164B and 166B of the passageways are spaced a distance D₈ which is greater than distance D₇ in this non-limiting embodiment. The outer portions of passageways 164 and 166 so spaced are configured to position pins 162A and 162B radially outwards so as to enable passage of distal end portion 545 and member 544 of plunger 54 therethrough as seen in FIG. 15.

Referring to FIG. 2, outer portions 164B and 166B of the passageways extend through wall 63 of pod housing 60 in this non-limiting embodiment. Referring to FIG. 5, applicator 34 in this non-limiting embodiment includes a pair of spaced-apart protrusions or tabs 165A and 165B positioned within interior 47 thereof. The tabs couple to and extend radially inwards from interior surface 44 of wall 42 thereof. Tabs 165A and 165B are generally rectangular prisms in shape in this non-limiting embodiment. The tabs are positioned between proximal end 36 and distal end 38 of applicator 34 in this non-limiting example. Tabs 165A and 165B are positioned to engage with outer portions 164B and 166B of passageways 164 and 166 of pod housing 60 when pod 58 is inserted into receptacle 52 of applicator 34. This configuration may function to facilitate insertion of the pod within the applicator in an aligned manner.

Referring back to FIG. 4, outer portions 164B and 166B of the passageways are in fluid communication with inner portions 164A and 166A of the passageways. Passageways 164 and 166 in this non-limiting example include transition or connector portions 164C and 166C that extend between and fluidly couple together the inner and outer portions thereof, respectively. The connector portions extend axially and laterally outwards and are slanted in this non-limiting example. Connector portions 164C and 166C of the passageways are axially positioned to coincide with the point at which needle 76 has been fully extended as seen in FIG. 14. As seen in FIG. 4, passageways 164 and 166 are thus configured such that the transition between inner portions 164A and 166A and outer portions 164B and 166B thereof corresponds to when the needle is fully extended. The passageways thus widen out from the inner to the outer portions thereof via connector portions 164C and 166C thereof, and allow distal end portion 545 of plunger 54 seen in FIG. 15 to push pins 162A and 162B radially outwards. This causes the space/opening or distance D₈ between the pins to increase and enables the plunger so biased by spring 56, to enter syringe assembly 70 and push on and move piston 72 axially forward towards distal end 64 of pod 58 so as to output medicament 68 from cartridge 65.

As seen in FIGS. 14 and 15, pins 162A and 162B are therefore moveable radially outwards upon needle 76 being fully extended so as to thereafter enable plunger 54 to pass therethrough thereafter. Thus during this transition of the pins moving to outer portions 164B and 166B of passageways 164 and 166 seen in FIG. 4, the pins are configured to move radially outwards so as to enable plunger 54 to fit therethrough. The spring-biased plunger is thus able to move from its retracted position seen in FIG. 11 to its extended position seen in FIG. 15. Plunger 54 so extended biases/pushes against piston 72 of cartridge 65, causing the piston to move towards forward end 69A of barrel 66, which in turn applies pressure to and causes medicament 68 to be expelled/ejected outwards from the cartridge/barrel, through needle 76 and into person 85. The plunger so extended thus biases the medicament outwards from the needle. Medicament 68 is thus biased outwards from the needle as seen in FIG. 15 subsequent to pod 58 being i) inserted into the receptacle as seen in FIG. 2; ii) moved from the locked protected needle pre-injection mode of FIG. 8 to an unlocked protected needle pre-injection mode seen in FIG. 11; and iii) then partially retracted mode seen in FIG. 13, which may be referred to as the beginning of the injection mode. Thus, at least front end portion 80 of pod 58 is spring-biased outwards from applicator 34 and configured selectively retract at least in part within the applicator to actuate release of the medicament. Syringe assembly 70 and/or cartridge 65 and barrel 66 thereof, are moveable and/or configured to be positioned towards, closer to or adjacent distal end 64 of pod 58 when medicament injector 30 is in the injection mode seen in FIG. 15. Needle 76 of the syringe assembly is extendable outwards from the distal end of the pod during the injection mode.

Still referring to FIG. 15, tabs 165A and 165B of applicator 34 are positioned to extend within outer portions 164B and 166B of passageways 164 and 166 in part so as to abut against and hold rearward barrier member 158 and thus pins 162A and 162B in place when the pins are positioned within the outer portions of the passageways. The above mechanism may ensure that needle 76 stays inside person 85 while the medication is being injected or pushed into the person. Tabs 165A and 165B and/or barrier member 158 may thus be referred to as a third coupling mechanism 167 which promotes positioning of needle assembly 74 in the deployed position until medicament 68 has been outputted from pod 58, with removable of inward pressure on forward end portion 80 of the pod thereafter enabling the pod to be automatically ejected from applicator 34 as described in further detail below.

Referring to FIGS. 15 and 17, once medicament injector 30 is pulled away from the person after fluid delivery, pod 58 is configured to be automatically ejected from receptacle 52 of applicator 34 thereafter following administration of the medicament. This occurs via spring 57 biasing the pod outwards from the applicator, with the pod (no longer held or locked in place via first and second coupling mechanisms 122 and 138) being ejected outwards from the front of the applicator as a result thereof.

Once medicament injector 30 is pulled away from the person after fluid delivery and referring to FIG. 16, syringe assembly 70 inside pod 58 is configured to be pushed back or biased axially rearward (as shown by arrow 168) towards proximal end 62 of the pod via spring 78 inside the pod. When this occurs, pins 162A and 162B are routed radially outwards and/or rearwards to outer portions 164B and 166B of passageways 164 and 166.

As seen in FIG. 4, passageways 164 and 166 include stops, protuberance, notches or catches 164D and 166D. Each catch is near or adjacent proximal end 62 of pod 58 in this non-limiting example. For passageways 164 and 166, catches 164D and 166D thereof may comprise zig-zag portions in fluid communication with outer portions 164B and 166B thereof in this non-limiting example. Referring to FIG. 3, the catches are radially inwardly positioned relative to exterior surface 63A of wall 63 of pod housing 60 in this example. Referring to FIG. 5, tabs 165A and 165B of applicator 34 engage outer portions 164B and 166B of passageways 164 and 166 at such a depth so as to slide under catches 164D and 166D, or slide past the catches via positions radially outwards from the catches when pod 58 is being inserted into applicator 34.

Pins 162A and 162B seen in FIG. 17 are biased via spring 78 towards proximal end 62 of pod 58 after the injection and/or use of the pod and upon removable of the pod from applicator 34. The pins so positioned are configured to lock in place via catches 164D and 166D seen in FIG. 4, with movement thereof thus being inhibited thereafter. Referring back to FIG. 17, pins 162A and 162B so positioned/locked, function inhibit pushing on rear end 69A of barrel 66 and/or inhibit deployment of needle 76 anew.

Thus, at the end of outer portions 164B and 166B of passageways 164 and 166, catches 164D and 166D seen in FIG. 16 trap pins 162A and 162B as seen in FIG. 17 and prevent further movement of the pins and therefore syringe assembly 70 (and/or cartridge 65 or barrel 66 and needle 76 thereof) upon pod 58 being removed or being in the process of being removed from applicator 34. This feature is configured to ensure that the needle is safeguarded after use to prevent needlestick injury from a person trying to push on the cartridge (and/or syringe and needle assembly) from the back or rear end 69A.

Cartridge-retaining arms 140A and 140B of second coupling mechanism 138, upon pod 58 being removed from applicator 34 after use, bias radially outwards anew as seen in FIG. 17 and are configured to inhibit re-insertion of the pod into the applicator once more. The following is a non-limiting embodiment which achieves this functionality.

Sleeve 152 seen in FIG. 16 may be configured to grip syringe assembly 70 (and/or cartridge 65 or barrel 66 thereof) at least in part even without being biased inwards via cartridge-retaining arms 140A and 140B of second coupling mechanism 138. Movement of syringe assembly 70 rearwards after the injection via spring 78 thus also causes the sleeve to move axially rearwards with the cartridge/barrel as pod 58 is removed or in the process of being removed from applicator 34. Catches 164D and 166D of passageways 164 and 166 seen in FIG. 16, when pins 162A and 162B seen in FIG. 17 couple thereto, are configured to inhibit further rearward movement of syringe assembly 70 (and/or cartridge 65, barrel 66 and needle 76 thereof) as well sleeve 152. The catches are also axially positioned such that cartridge-retaining arms 140A and 140B can no longer fully/effectively bias radially inwards so as to fit enable pod 58 to fit within receptacle 52 anew.

In this non-limiting example and as seen in FIG. 17, sleeve 152 is positioned rearward at least in part from distal end portions 144A and 144B of cartridge-retaining arms 140A and 140B. This end position of sleeve 152 results in the sleeve and cartridge-retaining arms no longer being aligned, with the cartridge-retaining arms being inhibited by the sleeve from sufficiently being able to pushed down and/or radially biased inwards once more. Attempting to insert pod 58 into receptacle 52 of applicator 34 anew may cause wall 42 of applicator 34 to abut cartridge-retaining arms 140A and 140B in part, which in turn may abut and/or jam against sleeve 152 so misaligned. This in turn may inhibit movement and/or jam against syringe assembly 70 (and/or cartridge 65 or barrel 66 thereof), inhibiting movement of the syringe assembly so rearwardly positioned and thus inhibiting the ability of pod 58 to be substantially fully inserted into the applicator anew and snap-fit together. This means that any used pods 58 will no longer be insertable into applicator 34 because they will no longer fit within receptacle 52 of the applicator 4. Cartridge-retaining arms 140A and 140B are thus configured to bias outwards and/or reduce compression of the sleeve in the injection mode of medicament injector 30 seen in FIGS. 14 and 15 as well as in post-injection mode seen in FIGS. 16 and 17. Sleeve 152 is thus axially spaced from cartridge-retaining arms 140A and 140B in the injection and post-injection modes seen in FIGS. 14, 15 and 17.

As seen with reference to FIGS. 15 and 17, when forward end portion 80 of pod 58 is no longer pushed/biased against skin 85A of person 85, spring 78 is configured to bias the forward end portion of the pod in the post-injection mode seen in FIG. 17 so as to cause the forward end portion of the pod to extend about. This functions to cover needle assembly 74 and needle 76 and inhibit access therethrough via the distal end 64 of the pod or otherwise. The post-injection mode seen in FIG. 17 may thus be referred to as a needle covered mode or position.

In summary, medicament injector 30 as herein described is configured and/or may thus be said to include a locked protected needle pre-injection mode seen in FIGS. 2 and 5 to 7, an unlocked protected needle pre-injection mode seen in FIGS. 9 to 12, an injection mode seen in FIGS. 13 to 15, and a post-injection mode seen in FIGS. 16 and 17. Each of these modes is described further below.

### Locked Protected Needle Pre-Injection Mode

In this mode seen in FIGS. 2 and 5 to 7, needle 76 is not active and cannot be triggered to extend out of pod housing 60 because pod cap assembly 82 is still locked onto pod 58. This mode includes every instance up until the pod is coupled to applicator 34 via first coupling mechanism 122 as seen in FIG. 8 and pod cap latch arms or male members 104A are deformed and “unlocked” from pod housing 60 as seen in FIG. 9.

### Unlocked Protected Needle Pre-Injection Mode

In this mode seen in FIGS. 9 to 12, the mechanism of injection is loaded, primed and/or live for injection but needle 76 is still physically shielded/covered by pod housing 60 and/or front end portion 80 of pod 58. This mode starts once pod cap assembly 82 is “unlocked” and ready to come off, and this mode ends when the front end portion of the pod housing is depressed to the point before arms 140A and 140B of second coupling mechanism 138 begin to move radially outwards along recessed wall 42 or recessed portions 146A and 146B of applicator 34.

### Injection Mode

In this mode seen in FIGS. 13 to 15, the injection mechanism is triggered, in this case by pressing forward end portion 80 of pod 58 against skin 85A of person 85 as seen in FIG. 13, and there is no longer any user input required. This mode starts right as second coupling mechanism 138 releases cartridge 65 (and/or syringe assembly 70 or barrel 66 thereof). This mode includes needle 76 entering the patient as seen in FIGS. 14 and 15 and the full dose of the medication or medicament 68 being biased outwards through the needle. This mode ends when the user input of removing applicator 34 away from the patient starts.

### Post-Injection Mode

This mode seen in FIGS. 16 and 17 starts when the applicator is being removed from the patient or person after full delivery of the medicament. This mode ends in a state where applicator 34 is set to receive another pod and needle 76 of the used or depleted pod 58 is shielded/covered and permanently locked in this shielded/covered configuration.

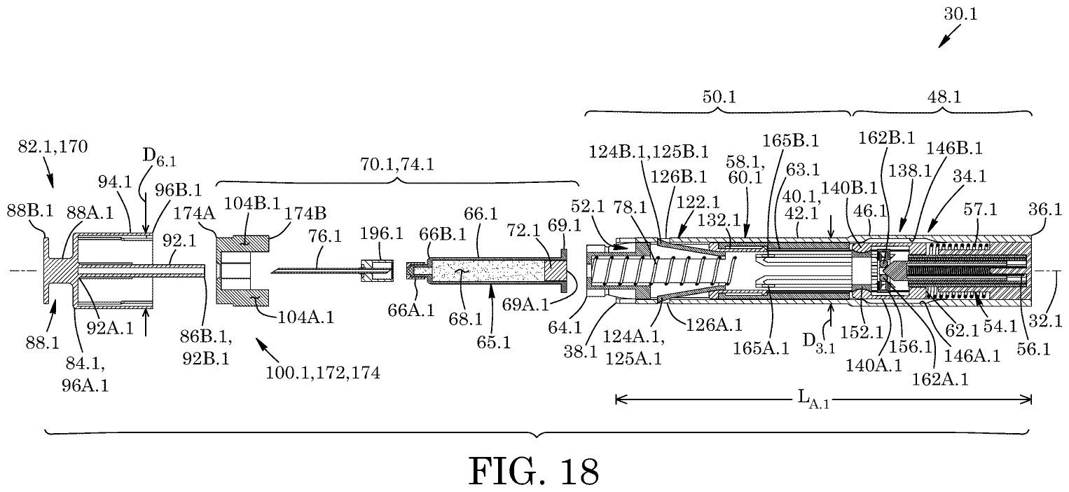

FIGS. 18 to 26 shows a medicament injector 30.1 according to another aspect. Like parts have like numbers and functions as medicament injector 30 shown in FIGS. 1 to 17 with the addition of decimal extension “0.1”. Medicament injector 30.1 is substantially the same as medicament injector 30 shown in FIGS. 1 to 17 with at least the following exceptions.

In this non-limiting embodiment, a barrier member, in this non-limiting embodiment a forward and/or reversible barrier member, in this case pod cap assembly 82.1 is configured to enable fluid communication between needle 76.1 and medicament 68.1 upon pod 58.1 being substantially inserted within receptacle 52.1 of applicator 34.1 according to one example. The pod cap assembly is configured to inhibit fluid communication between the needle and the medicament until the pod couples to the receptacle and/or applicator 34.1 according to another example. Pod cap assembly 82.1 is configured to inhibit fluid communication between needle 76.1 and medicament 68.1 until pod 58.1 is snap-fit connected to the receptacle and/or applicator according to a further non-limiting example. The pod cap assembly is configured to enable fluid communication between the needle and the medicament upon cap locking mechanism 100.1 moving to its unlocked mode. The following is a non-limiting embodiment which achieves this functionality.

As seen in FIG. 18, pod cap assembly 82.1 includes a first or outer portion, in this example an outer cap 170 and a second or inner portion, in this non-limiting example a locking member or inner cap 172. The inner cap of the pod cap assembly is configured to inhibit movement of the outer cap relative thereto and/or relative to needle assembly 74.1 until pod 58 is inserted within receptacle 52.1 of and/or couples to applicator 34.1 according to one non-limiting embodiment. Inner cap 172 is configured to inhibit movement of outer cap 170 relative thereto and/or relative to needle assembly 74.1 until the pod snap-fit couples to applicator 34.1 according to another non-limiting embodiment.

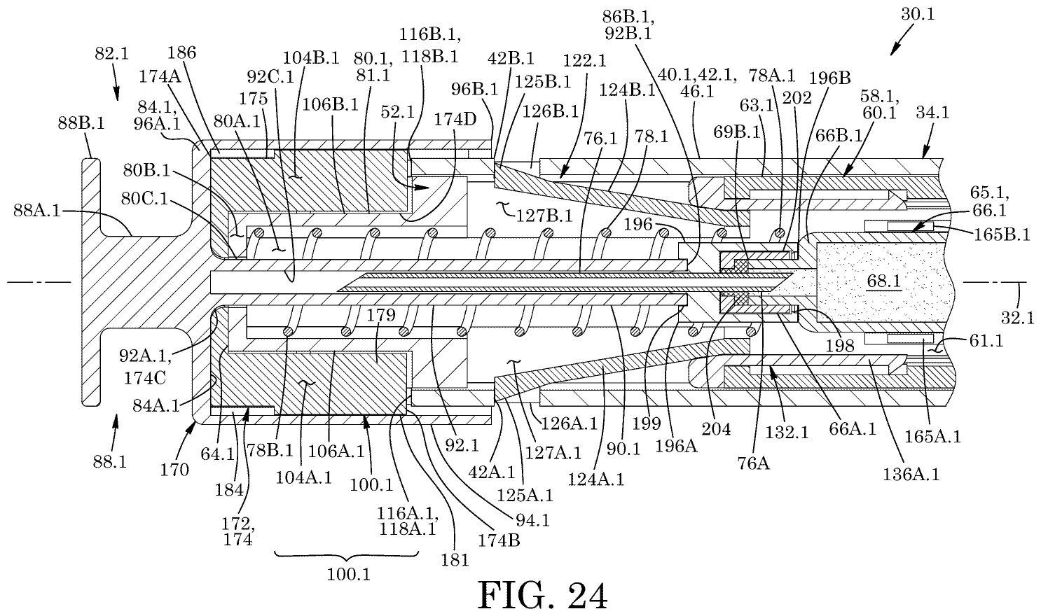

The inner cap of pod cap assembly 82.1 is moveable from a locked or extended position seen in FIG. 21, to an unlocked position seen in FIG. 24 upon pod 58.1 being inserted within and/or coupling to and/or snap-fit connecting with applicator 34.1. The pod cap assembly is configured such that movement of the inner cap of the pod cap assembly from the extended or locked position thereof seen in FIG. 21 to the retracted or unlocked position thereof seen in FIG. 24, enables needle 75.1 to pierce cartridge 65.1, in this case barrel 66.1, so as to be in fluid communication with medicament 68.1. The following are additional details of a non-limiting embodiment which achieve the above functionality.

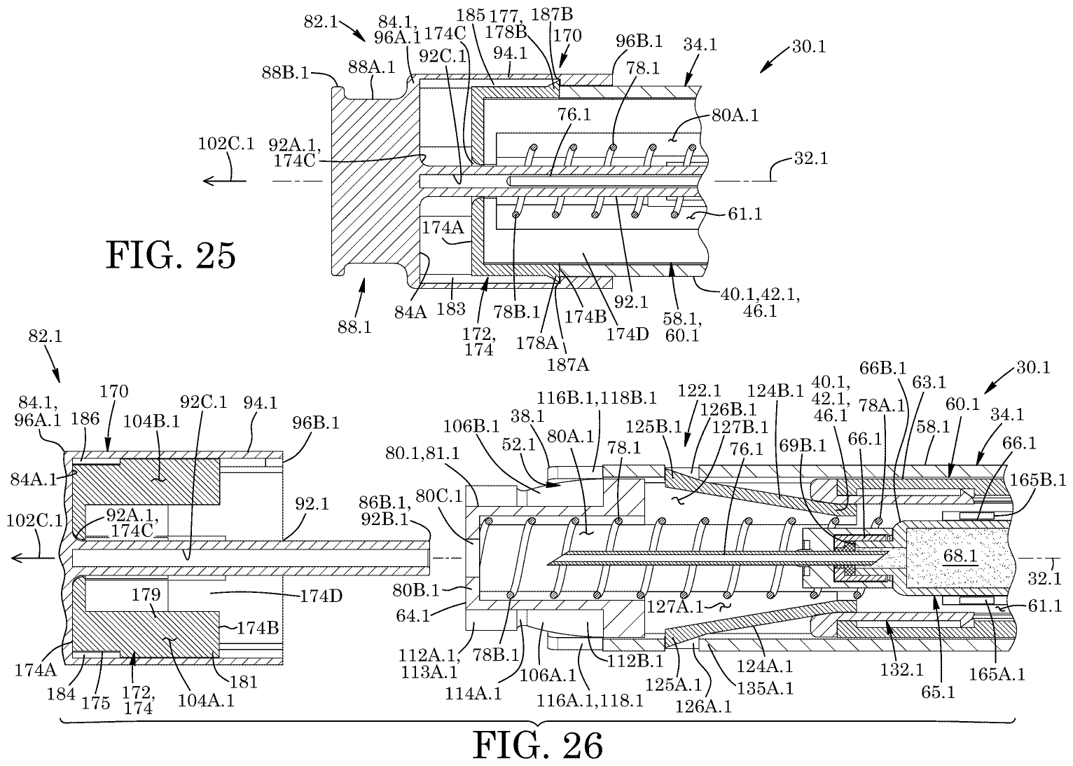

Pod cap assembly 82.1 is telescoping in this non-limiting example. As seen with reference to FIGS. 21 and 24, inner cap 172 slidably/telescopically couples to and is received within outer cap 170. Pod cap assembly 82.1 may thus be said to be divided into two telescoping members or components. As seen in FIG. 20, the inner cap includes a body 174. The body is substantially cylindrical in outer shape in this non-limiting example. Body 174 of inner cap 172 is coaxial with and extends about axis 32.1 in this example. As seen in FIG. 21, the body of the inner cap as a first, inner or closed end 174A and a second, outer or open end 174B axially spaced from the closed end thereof. Body 174 of inner cap 172 has an annular outer surface 175 which extends between the ends thereof. As seen in FIG. 25, a central aperture 174C extends longitudinally through closed end 174A of body 174 to enable inner sheath 92.1 to slidably extend therethrough. The central aperture is outwardly flared in this non-limiting example so as to enable the closed end of the body of inner cap 172 to abut closed end 92A.1 of the inner sheath and inner surface 84A.1 of cover member 84.1. Body 174 of the inner cap 172 has a bore 174D in fluid communication with central aperture 174C and which extends from open end 174B towards closed end 174A thereof. The bore and central aperture are coaxial with each other and axis 32.1 in this non-limiting example.

Referring back to FIG. 20, inner cap 172 includes circumferentially spaced-apart male members 104A.1 and 104B.1 of cap locking mechanism 100.1. The male members extend outwards in a direction extending from closed end 174A towards open end 174B of body 174. Male members 104.1 and 104B.1 extend axially outwards from open end 174B of the body of inner cap 172 in this non-limiting example. Recessed portions 102A.1 and 102B.1 of cap locking mechanism 100.1 align with the male members and extend through body 174 in a direction extending from open end 174B towards closed end 174A of the body.

Each male member 104A.1 includes an inner portion 179 which extends and faces radially inwards within bore 174D. The inner portions of the male members are shaped to selectively engage with female members 106A.1 and 106B.1 of pod 58.1 seen in FIG. 26 in a like manner as described above for medicament injector 30 seen in FIGS. 1 to 17. Referring back to FIG. 20, each male member 104A.1 includes an outer portion 181 which extends radially outwards at least in part from outer surface 175 of body 174 of inner cap 172.

Still referring to FIG. 20, inner cap 172 is configured to selectively couple to outer cap 170, in this example via a coupling mechanism 177 comprising a first of at least one male and female member, in this example in the form of a second pair of circumferentially spaced-apart male members 178A and 178B circumferentially spaced from male members 178A and 178B in this example. Male member 178A and 178B in this non-limiting example comprises or may be referred to as a catch. Male members 178A and 178B are resilient in part, in this example being configured to be selectively biased radially inwards as shown by arrow 180. This is achieved in one non-limiting example for each male member 178B via a pair of axially-extending and spaced-apart slots 182A and 182B extending radially through body 174 so as delineate the respective male member therefrom. The slots extend from adjacent closed end 174A towards open end 174B of the body.

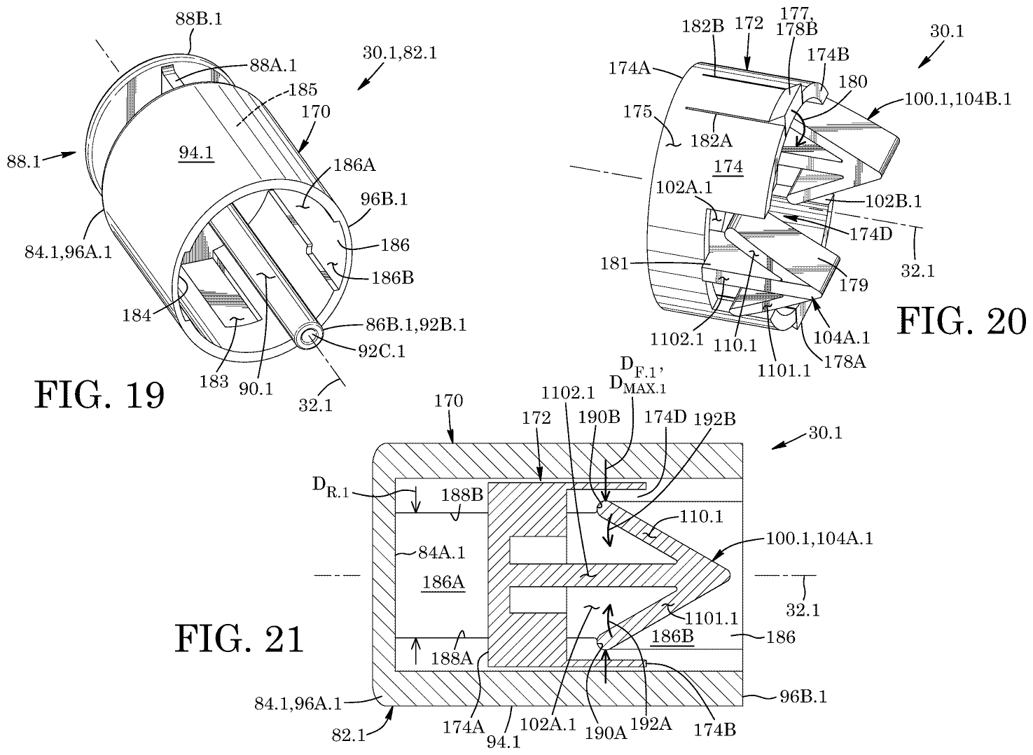

Referring to FIG. 25, outer cap 170 is configured to snap-fit connect to inner cap 172 in this non-limiting example. The following is a non-limiting embodiment which achieves this functionality. As seen in FIGS. 19 and 25, outer cap 170 comprises a second of at least one male and female member of coupling mechanism 177, in this example in the form of a first pair of circumferentially spaced-apart channels 183 and 185. Each channel is axially extending in this non-limiting example. As seen in FIG. 25, inner cap 172 is shaped to slidably engage with outer cap 170 via channels 183 and 185 and male members 178A and 178B. Insertion of the inner cap into open end 96B.1 of outer sleeve 94.1 causes male members 178A and 178B to abut the outer sleeve, with the male members being shaped/slanted to bias radially inwards as a result thereof until the male members reach channels 183 and 185, at which point the male members snap-fit/bias-outwards once more. Catches or shoulders 187A and 187B adjacent the outer, closed ends of the channels are shaped to inhibit passage of male members 178A and 178B therethrough anew. Inner cap 172 is thus inhibited from being removed from outer cap 170 upon being coupled thereto. First pair of channels 183 and 185 of the outer cap are thus shaped to engage with male members 178A and 178B of the inner cap such that removal of the outer cap promotes or causes removal of the inner cap as well.

Referring back to FIG. 19, outer cap 170 of pod cap assembly 82.1 has at least one and in this example a second pair of female members or channels 184 and 186. The second pair of channels are circumferentially spaced from the first pair of channels 183 and 185 seen in FIG. 25 in this non-limiting embodiment. As seen in FIG. 21, each of the second pair of channels is axially extending. Each channel 186 comprises one or more recessed portions: in this example a first recessed portion 186A and a second recessed portion 186B in fluid communication with the first recessed portion thereof. For each of the second pair of channels the first recessed portion thereof extends from cover member 84 towards open end 96B.1 of outer sleeve 94.1 of outer cap 170. Each first recessed portion 186A is elongate shaped to facilitate the passage of outer portion 181 of corresponding male member 104A.1 seen in FIG. 20 therein/therethrough: in this example and referring back to FIG. 21, each first recessed portion in this non-limiting embodiment comprises axially-extending and/or smooth opposed walls 188A and 188B in this non-limiting example and/or is rectangular in side profile. Each first recessed portion 186A spans a laterally-extending distance D_{R.1} which is less than the laterally-extending maximum distance D_{MAX.1} of elongate protrusions 110.1 and 1101.1 of male member 104A.1.

For each channel 186 each second recessed portion 186B is rectangular in side profile and enlarged compared to the first recessed portion thereof in this non-limiting embodiment. Each second recessed portion includes in this non-limiting embodiment at least one and in this example a pair of opposed or mirrored catches 190A and 190B or recessed cut-out grooves or recessed regions which form the same. The catches are shaped to fix positioning and/or inhibit retraction of inner cap 172 and/or male members 104A.1 and 104B.1 relative to outer cap 170 until pod 58.1 is inserted within, couples to and/or snap-fit connects with applicator 34.1. Each catch 190A is shaped to receive a distal end of a respective one of protrusions 110.1 and 1101.1 in this non-limiting embodiment, with each catch being outwardly concave in this non-limiting example. This functions to keep outer cap 170 in place relative to inner cap 172 with space separating cover member 84.1 of the outer cap and inner cap. Catches 190A and 190B are laterally spaced a distance D_{F.1} which generally or substantially equals to the laterally-extending maximum distance D_{MAX.1} of spanning distal ends of elongate protrusions 110.1 and 1101.1 of male member 104A.1.

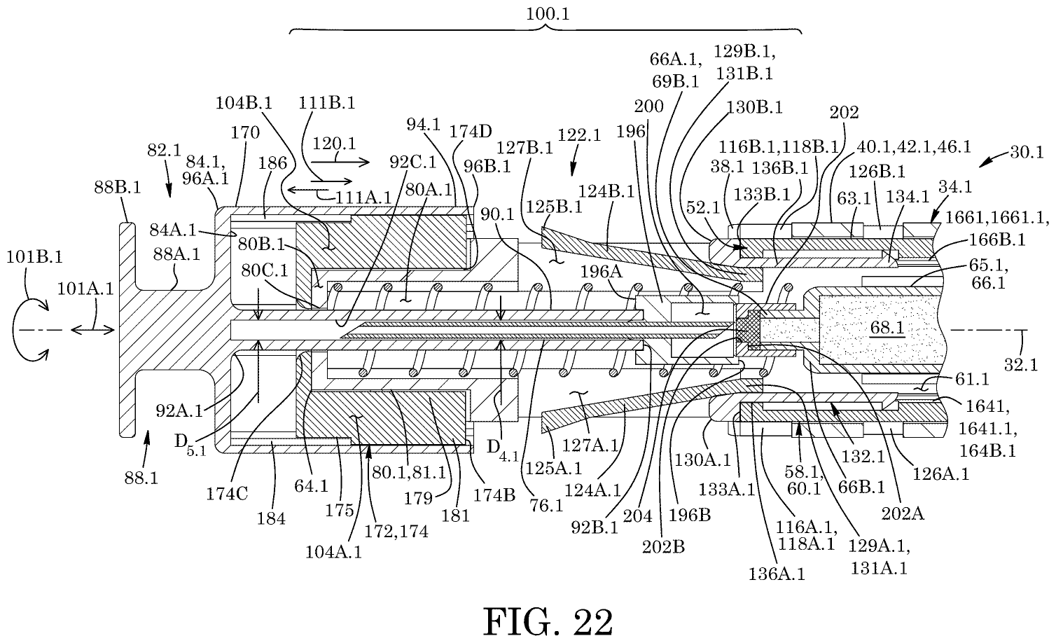

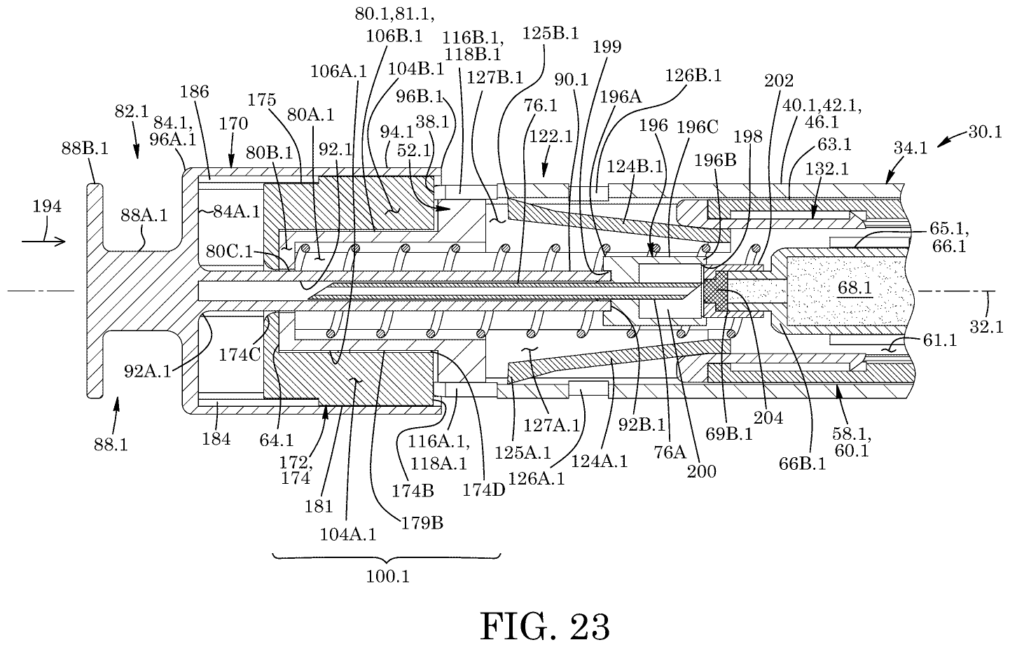

The second pair of channels 184 and 186 of outer cap 170 seen in FIG. 19 are shaped to slidably receive male members 104A.1 and 104B.1 seen in FIG. 20, with catches 190A and 190B seen in FIG. 21 promoting positioning of inner cap 172 in a locked or extended position. Referring to FIG. 22, pod cap assembly 82.1 couples to pod 58.1 via inner cap 172 and male members 104A.1/104B.1 and female members 106A.1 and 106B.1, and otherwise functions/operates in a like manner as described above for medicament injector 30 shown in FIGS. 1 to 17. Insertion of pod 58.1 with the cap assembly thereon, into applicator 30.1 as seen in FIG. 23, causes deflecting members 116A.1 and 116B.1 to dislodge corresponding male members 104A.1 and 104B.1 from their female member 106A.1 and 106B.1, which causes each male member to move from its locked to unlocked positions as described previously for medicament injector 30 shown in FIGS. 1 to 17. This additionally causes protrusions 110.1 and 1101.1 of male members 104A.1 seen in FIG. 21 to deflect radially inwards as seen by arrows 192A and 192B, which in turn causes outer portions 181 of the male members to dislodge from catches 190A and 190B of outer cap 170. This enables male members 104A.1 to slide into recessed portions 186A of channels 186 of the outer cap so as to enable inner cap 172 to move to its unlocked or retracted position seen in FIG. 24. Thus, when the “hook/harpoon” feature meets the slot groove of the applicator body, as before, the “hook/harpoon” arms or male members 104A.1 and 104B.1 are configured to bend towards each other inwards, which acts to release outer cap 170 from its position and allows the outer cap to telescopically move axially inwards in the direction of the “hook/harpoon” arms. This causes the outer cap including inner sheath 92.1 thereof, to effectively move/bias inwards towards pod 58 as shown by arrow 194 and with reference to FIGS. 23 and 24.

As seen in FIG. 22, cartridge 65.1 includes a cartridge cap 202 in this non-limiting example. The cartridge cap is cylindrical in outer shape in this non-limiting embodiment. Cartridge cap 202 is shaped to extend about and couple to neck 66A.1 and outer end 69B.1 of barrel 66.1. The outer or distal end of the cartridge cap and central aperture 202B thereof are covered/closed by a seal or seal member, in this example a septum 204 in this non-limiting example. The septum is pierceable and made of a resilient material, in this non-limiting example an elastomer. Septum 204 spans outer end 69.1 of barrel 66.1 so as to close the opening thereof. The septum is shaped to fit between an inner end or seat or annular shoulder 202A of cartridge cap 202 and the outer end of the barrel in this non-limiting embodiment. Cartridge 65.1 may be defined in this non-limiting embodiment as comprising: barrel 66.1; piston 72.1 slidably/sealably positioned within the barrel thereof; cartridge cap 202 coupled to the outer/distal end of the barrel thereof; and septum 204 coupling to and spanning the opening of the cartridge cap thereof. Syringe barrels, pistons, cartridge caps and septums including their various parts and functionings thereof, are known per se and barrel 66.1, piston 72.1, cartridge cap 202 and septum 204 will accordingly not be described in further detail.

As seen in FIG. 23, needle assembly 74.1 includes a double ended needle 76.1 in this non-limiting embodiment. The needle assembly is configured to inhibit communication between needle 76.1 thereof and medicament 68.1 until pod 58.1 is inserted within, coupled to and/or snap-fit connected to receptacle 52.1 and/or applicator 34.1. Needle assembly 74.1 is telescoping in this non-limiting example.

The needle assembly includes in this non-limiting example a first or outer hub 196, which is tubular in this non-limiting example. The outer hub is shaped to couple to inner sheath 92.1. Outer hub 196 in this non-limiting example is shaped receive and/or couple to open end 92B.1 of inner sheath 92.1 via a first or outer connector thereof, in this example an annular recessed portion 199 adjacent a first or distal end 196A thereof. The outer hub includes a protuberance, in this non-limiting example an annular, radially-inwardly extending catch 198 adjacent a second or proximal end 196B thereof. The outer hub has a bore 200 extending from proximal end 196B towards distal end 196A thereof. Outer hub 196 has an outer surface 196C which is annular in this non-limiting example and which extends between ends 196A and 196B thereof. The outer hub is shaped to receive inner end portion or inner needle tip 76A of needle 76 therethrough, with the bore of the outer hub being shaped to extend about the inner needle tip of the needle.

Outer hub 196 of the needle assembly is shaped to receive cartridge cap 202 within bore 200 thereof upon pod 58.1 being inserted within, coupled to and/or snap-fit connected to receptacle 52.1 and/or applicator 34.1 as seen in FIG. 24. Inward movement of outer cap 170 as seen by arrow 194 in FIG. 23, causes inner sheath 92.1 to bias outer hub 196 towards the cartridge cap so as to overcome friction and/or mechanical interference caused by catch 198, thereby enabling the cartridge cap to be received within bore 200 of the outer hub. Outer hub 196 snap-fit couples to the cartridge cap in this non-limiting example with catch 198 thereof abutting shoulder 66B.1 of barrel 66.1 and functioning to inhibit removal of the outer hub from the cartridge cap thereafter. At resting state/storage, double ended needle 76.1 and outer hub 196 are configured to friction or interference fit to inner sheath 92.1 and/or cartridge cap 202.

The outer hub of needle assembly 74.1 thus telescopically receives the cartridge cap and this causes inner needle tip 76A of needle 76.1 to pierce septum 204 of barrel 66.1 as seen in FIG. 24. Fluid communication between the needle and medicament 68.1 is thus enabled. This movement of outer cap 170 means that needle cover or inner sheath 92.1 pushes double-ended needle 76.1 onto cartridge 65.1 or barrel 66.1 or outer cap or septum. Simultaneously, the inner smaller needle or inner needle tip 76A of needle 76.1 is configured to puncture the cartridge/barrel or outer cap or septum 204 and become in fluid communication with medicament 68.1 within the barrel.

As seen in FIG. 26, pod cap assembly 82.1 is simultaneously unlocked. Outer cap 170 may thus then be removed as shown by arrow 102C.1, which causes shoulders 187A and 187B of the outer cap seen in FIG. 25 to abut male members 178A and 178B of inner cap 172, thereby enabling the inner cap to move effectively as one with the outer cap and also thus be removed from pod 58.1 and applicator 30.1. Thus, when pod cap assembly 82.1 is unhooked from the pod, pod cap assembly 82.1 is configured to enable the user to then pull the pod cap assembly off and/or outwards/away from the pod and in doing so, outer cap 170 catches on inner cap 172, allowing both the outer cap and inner cap to come off or be removed from the pod together or as one. Medicament injector 30.1 as herein described is thus configured such that: i) at no point before removal of pod cap assembly 82.1 will needle 76.1 be exposed to the outside; ii) the pod cap assembly is only removable upon pod 58.1 being inserted into, coupling with and/or snap fitting connecting to applicator 34.1; and iii) needle assembly 74.1 is only in fluid communication with medicament 68.1 upon the pod being inserted into, coupling with and/or snap fitting connecting to the applicator and outer cap 170 being pushed axially inwards towards the pod/applicator. One or more of these configurations/functionalities enabled by the non-limiting embodiment as herein described, may be critical for maintaining sterility of the needle and corresponding fluid path of medicament 68.1.

Pod cap assembly 82.1 and/or needle assembly 74.1 may be referred to as a first telescoping mechanism. Applicator 34.1 may thus be said and/or shaped to receive pod 58.1 and actuate said first telescoping mechanism thereby to cause needle assembly 74.1 to pierce cartridge 65.1 or barrel 66.1 thereof. Drive element or plunger 54.1 may be referred to as a second telescoping mechanism, with actuation of which thereafter promoting outputting of medicament 68.1 from needle 76.1. The injection process is otherwise substantially to that described for medicament injector 30 shown in FIGS. 1 to 17.

FIGS. 27 to 43B shows a medicament injector 30.2 according to a further aspect. Like parts have like numbers and functions as medicament injector 30.1 shown in FIGS. 18 to 26 with decimal extension “0.2” replacing decimal extension “0.1” and being added for parts not previously having a decimal extension. Medicament injector 30.2 is substantially the same as medicament injector 30.1 shown in FIGS. 18 to 26 with at least the following exceptions.

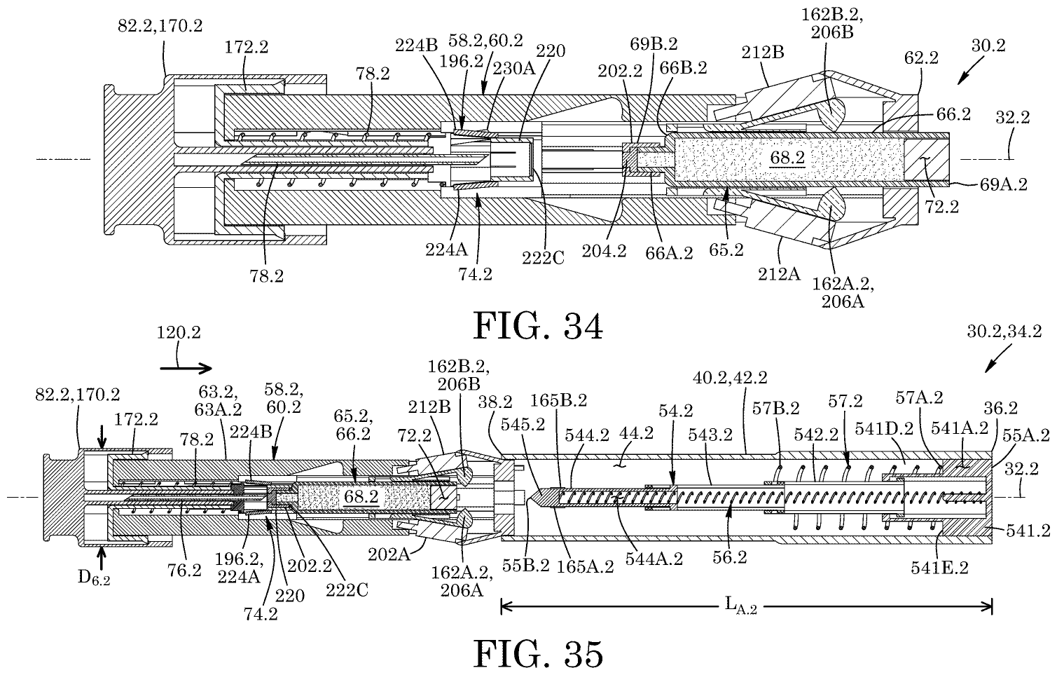

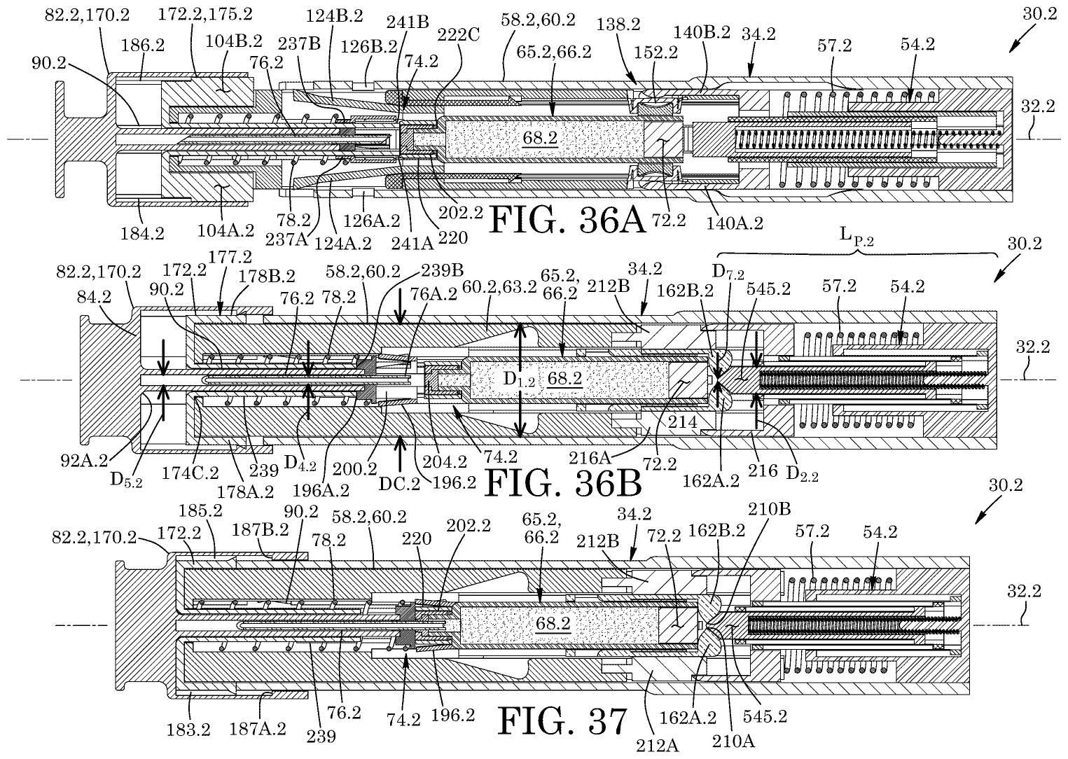

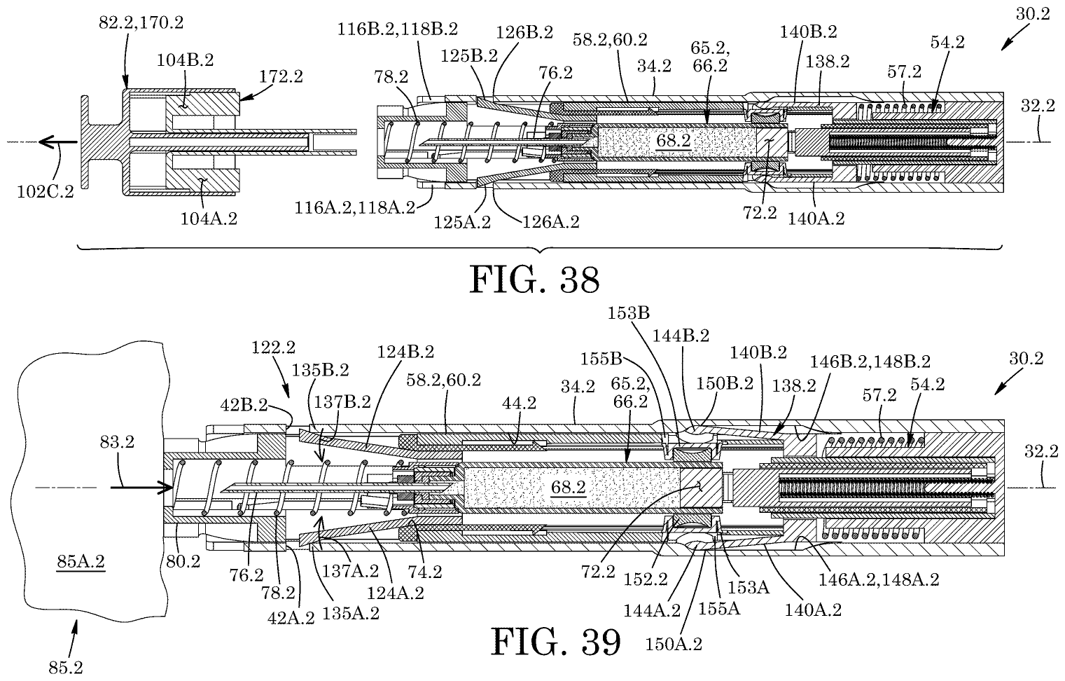

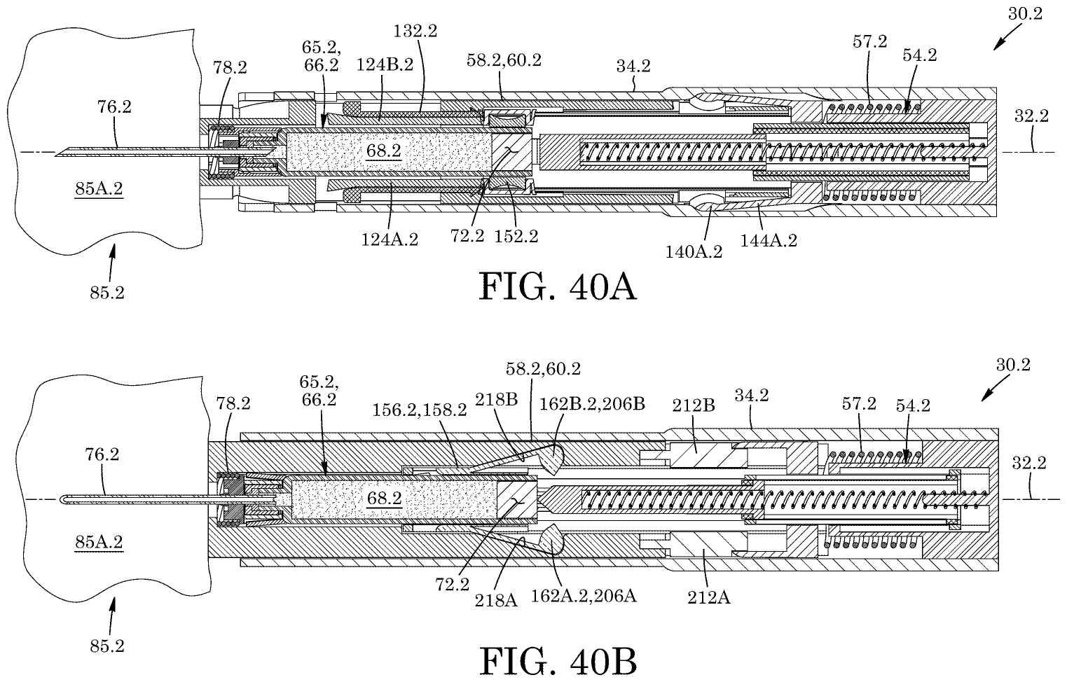

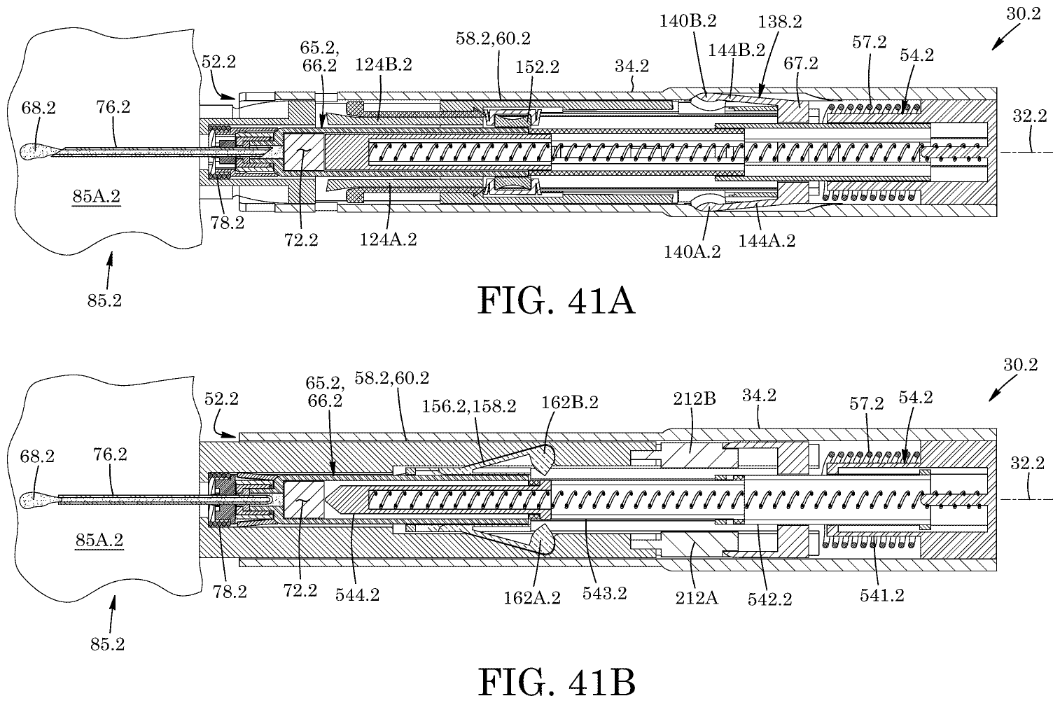

As seen in FIGS. 34, 35 and 36B, pod 58.2 is shaped to selectively receive an interchangeable cartridge 65.2 which is insertable separately by a medical or non-medical person. The pod in this non-limiting example is user-insertable. Thus, pod 58.2 in this non-limiting embodiment may be provided/sold with or without cartridge 65.2. The cartridge may be purchased separately and/or comprise a third party product, for example. As seen in FIG. 34, proximal end 62.2 of pod 58.2 is configured to receive user-insertable cartridge 65.2 therein in this example. The pod is thereafter loadable into applicator 34.2 seen in FIG. 35 so as to be inserted therein, couple thereto and/or snap fit connect therewith, and prime the drive element or plunger 54.2 as seen in FIG. 36B. Cap assembly 82.2 may then be removed as shown in FIG. 38. Referring to FIG. 39, partial retraction of pod 58.2 thereafter into applicator 34.2, in this example by pressing forward end portion 80.2 of the pod against person 85.2, functions to actuate/release/fire plunger 54.2 to engage the pod as seen in FIGS. 40B and 41B so as to output medicament 68.2 automatically therefrom. The following particulars provide non-limiting additional details which achieve this functionality.

As seen in FIG. 31C, pod 58.2 includes at least one and in this example a pair of spaced-apart protrusions, in this non-limiting example in the form of a pair cartridge-receiving arms 162A.2 and 162B.2. The cartridge-receiving arms may be referred to as inner arms. Cartridge-receiving arms 162A.2 and 162B.2 are configured to receive cartridge 65.2 therethrough as seen in FIG. 34 and thereafter inhibit access to the cartridge at least initially and/or in the first instance as seen in FIG. 36B. Referring back to FIG. 31C, the cartridge-receiving arms in this non-limiting example couple to and extend radially-outwards at least in part from body 160A.2 of second or rearward barrier member 158.2.

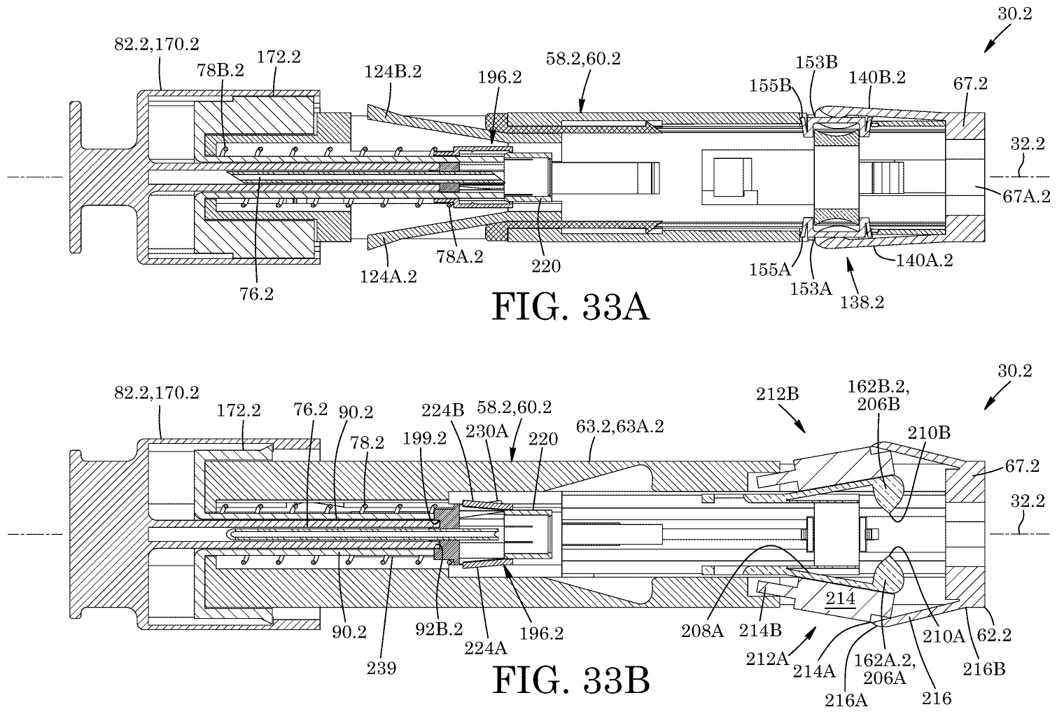

Sleeve 152.2 couples to body 160A.2 of rearward barrier member 158.2 in this non-limiting example. As seen in FIG. 33A, the sleeve and rearward barrier member couple to pod housing 60.2 via a pair of mounts or brackets 153A and 153B which extend about the exterior of circumferentially spaced portions of the sleeve and fit within radially-extending recesses or apertures 155A and 155B of the housing. Once the brackets get pushed down onto sleeve 155.2, the brackets are configured to stay on the sleeve and move with the sleeve for the duration of the deployment cycle of medicament injector 30.2. Brackets 153A and 153B are resilient in this example and configured to hold sleeve 152.2 and rearward barrier member 158.2 in place in a first instance

Referring back to FIG. 31C, cartridge-receiving arms 162A.2 and 162B.2 include distal end portions 206A and 206B which are enlarged and/or bulbous in this non-limiting example. The cartridge-receiving arms include elongate portions 208A and 208B via which the distal end portions thereof connect to body 160A.2 of rearward barrier member 158.2: in this non-limiting embodiment the cartridge-receiving arms and body all make up the one piece that is rearward barrier member 158.2 and thus may be said to be integrally connected so as to form a unitary whole. The elongate portions are rectangular prisms in shape in this non-limiting example. Arms 162A.2 and 162B.2 are configured such that distal end portions 206A and 206B thereof are biased radially-outwards. As seen in FIG. 33B, arms 162A.2 and 162B.2 extend rearwards or towards proximal end 62.2 of pod 58.2 in this non-limiting example. Distal end portions 206A and 206B of the cartridge-receiving arms are shaped to extend and/or point radially-inwards in this example via tapered or pointed ends 210A and 210B in this non-limiting embodiment.

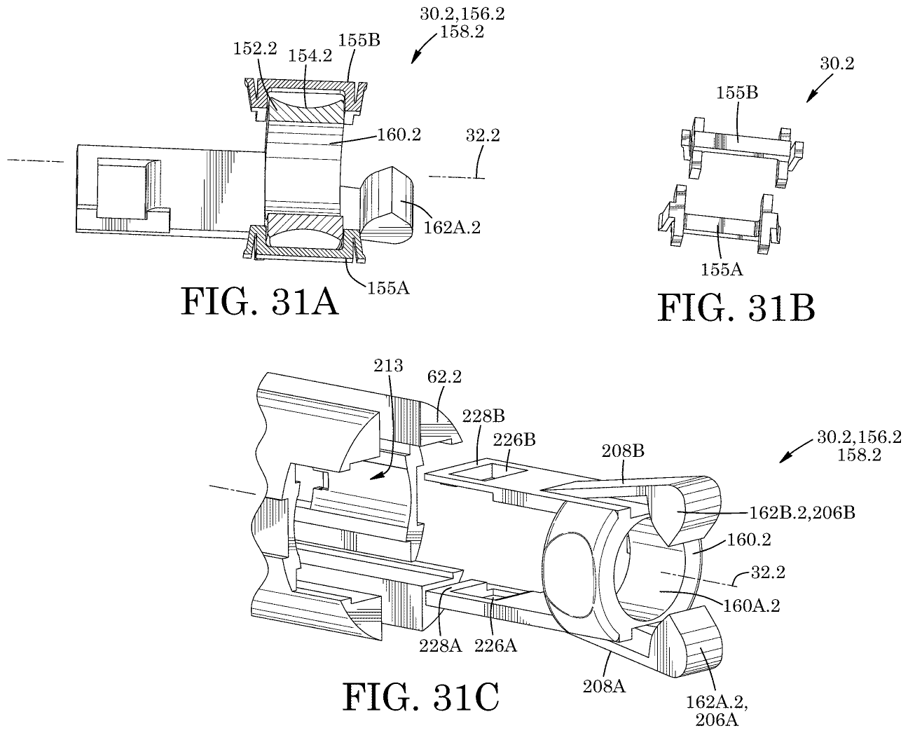

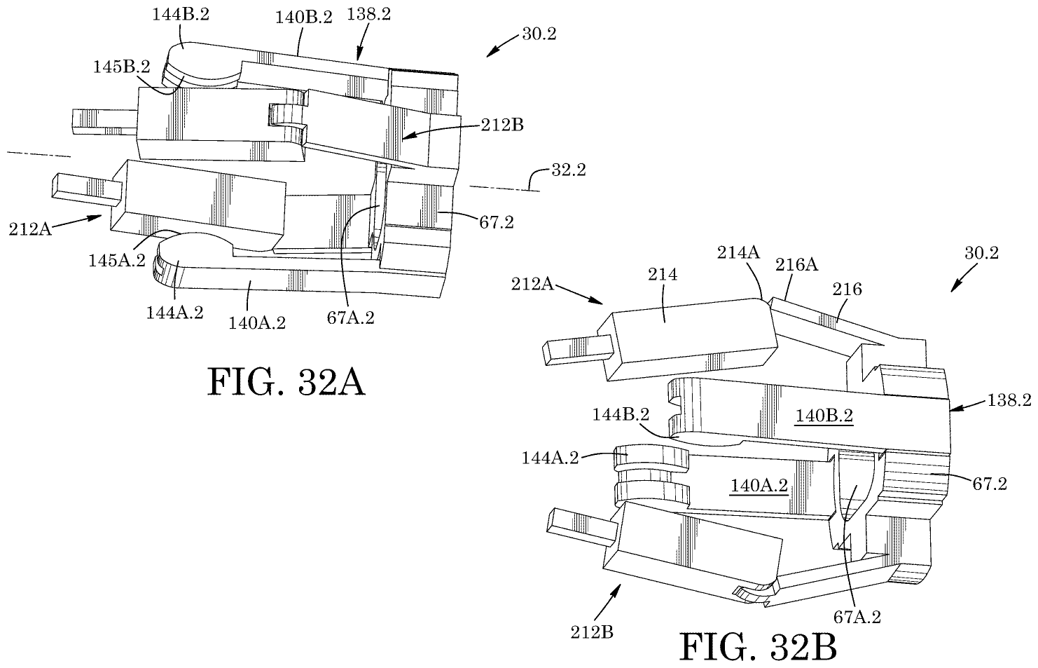

As seen in FIG. 32B, pod 58.2 includes one or more deflecting members, in this example a pair of hinged outer arms 212A and 212B; however the latter is not strictly required and the arms and/or deflecting members may be non-hinged in other embodiments. The outer arms couple to and are circumferentially spaced from cartridge-retaining arms 140A.2 and 140B.2 in this non-limiting example via a base or insert 67.2 of pod 58.2; however, here too this is not strictly required. The insert has a bore 67A.2 shaped to selectively receive cartridge 65.2 therethrough. Insert 67.2 is shaped to mate with a corresponding recessed portion or opening 213 of pod 58.2 adjacent proximal end 62.2 of the pod as seen in FIG. 31C so as to enable a specific/preferred/aligned positioning of arms 140A.2, 140B.2, 212A and 212B relative to the pod and so as to dislodgement thereof thereafter. In this non-limiting example the insert of the pod seen in FIG. 32B is cross-shaped in outer profile and the opening of the pod has a corresponding female cross-shape configured to receive the same; however, this configuration/shape is not strictly required.

Referring back to FIG. 35, outer arms 212A and 212B are configured to radially bias outwards at least in part from pod housing 60.2, in this example extending outwards from exterior surface 63A.2 of wall 63.2 when pod 58.2 is free-of/removed-from applicator 34.2. Referring back to FIG. 32B, each outer arm 212A includes in this non-limiting example first and second elongate members 214 and 216 which are hingedly connected together at adjacent ends 214A and 216A thereof. Each elongate member is a rectangular prism in shape in this non-limiting embodiment, with the first elongate member being thicker and shaped to function as part of or an extension of wall 63.2 of pod housing 60.2 when the arms 212A and 212B are in retracted positions seen in FIG. 36B. As seen in FIG. 33B, for each arm 212A first elongate member 214 pivotally connects to the wall of the pod housing via an outer end 214B thereof and second elongate member 216 is moveable relative and connects via outer end 216B to insert 67.2 of pod 58.2. In this non-limiting example, the second elongate members are integrally connected to the insert of the pod so as to form a unitary whole. Inner/adjacent ends 214A and 216A of the first and elongate member hingedly connected together. First elongate members 214 of outer arms 212A and 212B are shaped to extend along abut cartridge-receiving arms 162A.2 and 162B.2 (in this example elongate portions 208A thereof).

As seen in FIG. 34, to load cartridge 65.2 into pod 58.2, forward end 69B.2 of barrel 66.2 is in this example manually inserted into the pod via insertion or proximal end 62.2 of the pod. This causes cartridge-receiving arms 162A.2 and 162B.2 via distal end portions 206A and 206B to abut the barrel and bias radially outwards, which in turn biases outer arms 212A and 212B radially outwards. As seen in FIG. 35, distal end portions 206A and 206B of the cartridge-receiving arms are positioned rearwards of cartridge 65.2 once the cartridge is fully inserted into place within pod 58.2.

The pod is now ready to be inserted into applicator 34.2. As seen with reference to FIGS. 34 and 36B, insertion of pod 58.2 into applicator 34.2 causes outer arms 212A and 212B to abut wall 42.2 of outer body 40.2 of applicator 34 and bias radially inwards relative thereto. The outer arms so biased inwards cause cartridge-receiving arms 162A.2 and 162B.2 to bias radially inwards so as to inhibit access to cartridge 65.2 when pod 58.2 is inserted into applicator 34.2. As seen in FIG. 36B, when the pod is fully inserted into the applicator, distal end portions 206A and 206B of cartridge-receiving arms 162A.2 and 162B.2 are configured to be spaced adjacent to each other, with pointed ends 210A and 210B thereof separated by distance D_{7.2}. The distal end portions of the cartridge-receiving arms so positioned are configured to inhibit distal end portion 545.2 of plunger 54.2 from passing therethrough and abutting piston 72.2 of cartridge 65.2.

Cartridge-receiving arms 162A.2 and 162B.2 are configured to inhibit the plunger from engaging the cartridge until needle 76.2 or needle assembly 74.2 are moved to their deployed position as seen with reference to FIGS. 39 and 40B. In this non-limiting embodiment the cartridge-receiving arms are configured to move radially outwards so as to enable plunger 54.2 to extend therepast in the deployed position of the needle or needle assembly. In this non-limiting example the cartridge-receiving arms are received within radially outwardly extending recessed portions 218A and 218B of pod 58.2 in the deployed position of the needle or needle assembly as seen in FIG. 40B. The recessed portions are shaped to mate with and receive arms 162A.2 and 162B.2 so as to cause the distance between distal end portions 206A and 206B to enlarge so as to enable passage of plunger 54.2 therethrough.

The cartridge-receiving arms are thus configured to enable deployment of needle 76.2 or needle assembly 74.2 outwards from distal end 64.2 of pod housing 60.2 of pod 58.2 in a first instance. Retraction of pod 58.2 into applicator 34.2 by pressing against person 85.2 as seen in FIG. 39 and arrow 83.2, causes cartridge-retaining arms 140A.2 and 140B.2 to move within recessed portions 146A.2 and 146B.2 of applicator 34.2, thereby reducing pressure on sleeve 152.2 as described above. Movement of cartridge 65.2 forwards via plunger 54.2 thereafter causes brackets 155A and 155B to dislodgement from apertures 155A and 155B and enables rearward barrier member 158.2 to move forwards.

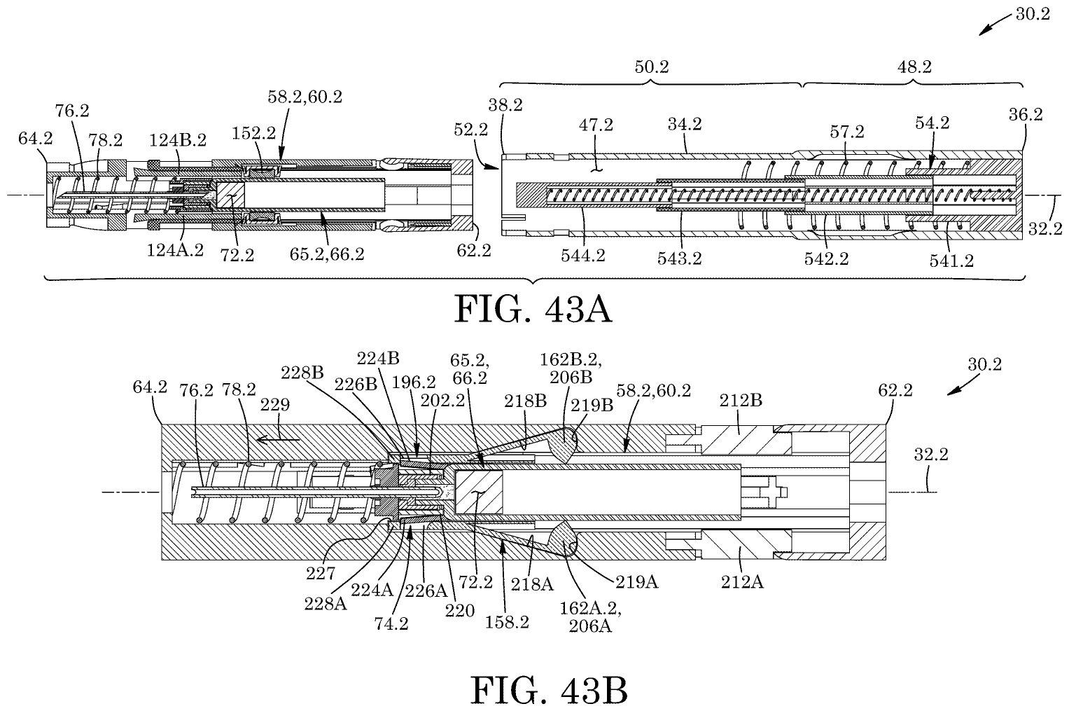

Cartridge-receiving arms 162A.2 and 162B.2, upon needle 76.2 or needle assembly 74.2 moving back towards a covered or retracted position seen in FIG. 43B, are configured to inhibit deployment of the needle or needle assembly outwards therefrom thereafter. A non-limiting embodiment which achieves this functionality is discussed in further detail below.

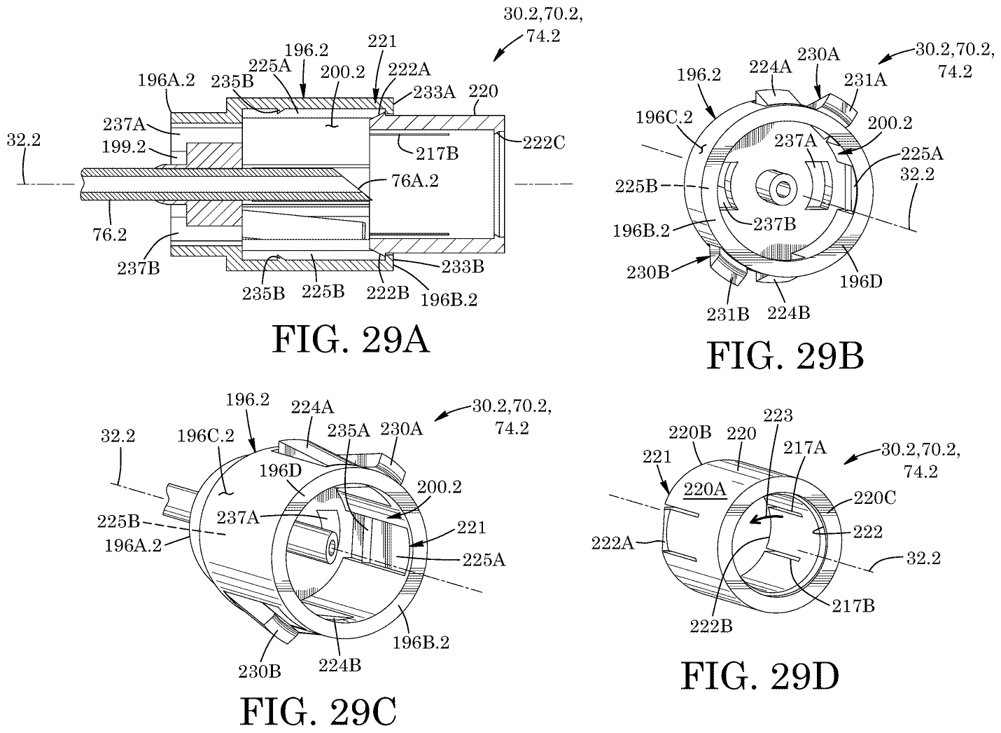

As seen in FIG. 29A, needle assembly 74.2 is telescoping. The needle assembly includes an outer hub 196.2 and an inner hub 220 slidable relative to the outer hub thereof. The inner hub is configured to selectively couple to the outer hub, in this example via a coupling mechanism 221 comprising a first of at least one male and female member, in this example in the form of a pair of circumferentially spaced-apart male members 222A and 222B seen in FIG. 29D. Each male member in this non-limiting example comprises or may be referred to as a catch, in this non-limiting example being angled/slanted at least in part relative to axis 32.2 and in this non-limiting embodiment being triangular or wedge shaped at least in part in lateral section. Male members 222A and 222B are resilient in part, in this example being configured to be selectively biased radially inwards as shown by arrow 223. This is achieved in one non-limiting example for each male member 222B via a pair of axially-extending and spaced-apart slots 217A and 217B extending radially through body 220A of inner hub 220 so as delineate the respective male member therefrom. The slots extend from adjacent a first or distal end 220B towards a second or proximal end 220C of the body. Inner hub 220 includes a second male member or catch 222C adjacent the proximal end of the body. Catch 222C is annular in this non-limiting embodiment, radially inwardly-extending in this non-limiting example, angled/slanted at least in part relative to axis 32.2 and in this non-limiting embodiment triangular or wedge shaped at least in part in lateral section.

Referring to FIG. 29A, outer hub 196.2 is configured to snap-fit connect to inner hub 220 in this non-limiting example. The following is a non-limiting embodiment which achieves this functionality. Outer hub 196.2 comprises a second of at least one male and female member of coupling mechanism 221, in this example in the form of a first pair of circumferentially spaced-apart channels 225A and 225B. Each channel is axially extending in this non-limiting example. As seen in FIG. 29A, inner hub 220 is shaped to slidably engage with outer hub 196.2 via channels 225A and 225B and male members 222A and 222B. Insertion of the inner hub into proximal end 196B.2 of outer hub 196.2 causes male members 222A and 222B to abut the outer hub. The male members are shaped/slanted to bias radially inwards as a result thereof until the male members reach channels 225A and 225B, at which point the male members snap-fit/bias-radially-outwards once more.

As seen in FIG. 29A, outer hub 196.2 includes a first or outer pair of shoulders or stops 233A and 233B in fluid communication with channels 225A and 225B, respectively. The stops are radially-extending and adjacent proximal end 196B.2 of the outer hub in this non-limiting example. Stops 233A and 233B are shaped to inhibit passage of male members 222A and 222B therepast anew. Inner hub 220 is thus inhibited from being removed from outer hub 196.2 upon being coupled thereto.

Still referring to FIG. 29A, outer hub 196.2 includes a second pair of shoulders or stops 235A and 235B positioned between ends 196A.2 and 196B.2 thereof and in fluid communication with channels 225A and 225B, respectively. The stops are angled/slanted at least in part relative to axis 32.2 in this non-limiting example, in this case being triangular or wedge shaped in lateral section.

As seen with reference to FIG. 35, inner hub 220 of needle assembly 74.2 is shaped in this non-limiting embodiment to couple to cartridge 65.2 so user-inserted into pod 58.2, in this non-limiting example snap-fit coupling thereto. Catch 222C is shaped in this non-limiting embodiment to extend about and couple to cartridge cap 202.2 of cartridge 65.2 so user-inserted into pod 58.2 so as to inhibit removal of the cartridge thereafter as seen in FIG. 36A. The pod is configured to make an audible sound or click to indicate when the cartridge so user-inserted is properly locked into place in this non-limiting embodiment.

As seen in FIG. 37, outer hub 196.2 of needle assembly 74.2 is shaped to more fully receive and extend about inner hub 220 of needle assembly 74.2 upon pod 58.2 being inserted within applicator 34.2 and outer cap 170.2 being biased axially inwards relative to inner cap 172.2. The outer hub is shaped to receive the inner hub upon the pod being coupling to the applicator in this non-limiting embodiment, in this example upon the pod or cartridge assembly snap-fit connecting to the applicator. As seen in FIG. 29A, male members 222A and 222B of inner hub 220 are shaped to slide past stops 235A and 235B of outer hub 196.2 so as to snap-fit couple thereto in a retracted position of the inner hub relative to the outer hub. The male members and catches are shaped to inhibit removal of the inner hub from its retracted position thereafter.

As seen in FIG. 29A, outer hub 196.2 has in this non-limiting example one or more and in this example a pair of circumferentially spaced-apart apertures 237A and 237B extending axially through from distal end 196A.2 towards proximal end 196B.2 thereof. The apertures align with channels 225A and 225B in this non-limiting example; however this is not strictly required.

As seen in FIG. 28, in this non-limiting example locking member or inner cap 172.2 of cap assembly 82.2 includes an inner sleeve or sheath 239 connected to body 174.2 thereof and extending about axis 32.2: in this non-limiting embodiment, the inner sheath integrally connects to the body so as to form a unitary whole. The inner sheath of the inner cap is tubular in this non-limiting example. As seen with reference to FIGS. 33B and 37, inner sheath 239 of inner cap 172.2 is shaped to extend about and slidably move telescopically relative to inner sheath 90.2 of outer cap 170.2. Referring back to FIG. 28, the inner sheath of the inner cap has a first or proximal end 239A coupled to body 174.2 of the inner cap and a second or distal end 239B spaced-apart from the proximal end thereof. As seen in FIG. 36B, distal end 239B of inner sheath 239 of inner cap 172.2 is configured to abut distal end 196A.2 of outer hub 196.2 of needle assembly 74.2.

Referring back to FIG. 28, the inner sheath of the inner cap in this non-limiting embodiment includes one or more and in this example a pair of circumferentially spaced-apart distal end portions or protrusions 241A and 241B. As seen in FIG. 36A, the protrusions are shaped to extend through apertures 237A and 237B of outer hub 192.2 of needle assembly 74.2 and function as one or more end stops for cartridge 65.2 so user-inserted. Protrusions 241A and 241B are configured to abut cartridge cap 202.2. Inner cap 172.2 of cap assembly 82.2 so shaped thus functions to inhibit cartridge 65.2 from moving longitudinally/axially therepast. The inner cap of the cap assembly (including the protrusions thereof) extends axially inwards past and about inner needle tip 76A.2 of needle assembly 74.2 in this non-limiting example. Cap assembly 82.2 as herein described is thus shaped to inhibit premature puncturing of cartridge 65.2 when the cartridge is user-inserted into pod 58.2.

Referring back to FIGS. 29B and 29C, outer hub 196.2 of needle assembly 74.2 includes one or more radially outwardly-extending first elongate members or prongs 224A and 224B. The first prongs are configured to enable deployment of needle 76.2 or needle assembly 74.2 outwards from distal end 64.2 of pod housing 60.2 of pod 58.2 in a first instance. As seen in FIG. 43B, first prongs 224A and 224B, upon the needle or needle assembly moving back to a covered or retracted position, are configured inhibit deployment of the needle or needle assembly outwards therefrom thereafter. The following is a non-limiting embodiment which achieves this functionality.

As seen in FIGS. 29B and 29C, first prongs 224A and 224B are resilient and elongate. The first prongs are biased to extend radially outwards, extending outwards at least in part from outer surface 196C.2 of outer hub 196.2. First prongs 224A and 224B are rectangular prisms in shape in this non-limiting embodiment; however, this is not strictly required. As seen in FIG. 43B, the first prongs extend towards distal end 64.2 of pod 58.2 at least in part in this non-limiting embodiment. The first prongs are circumferentially spaced from channels 225A and 225B in this example. As seen in FIG. 43B, first prongs 224A and 224B are configured to selectively couple with rearward barrier member 158.2 in this non-limiting embodiment, upon the needle or needle assembly moving back to a covered or retracted position. The first prongs snap fit connect to the rearward barrier member in this non-limiting example. Rearward barrier member 158.2 includes one or more and in this example a pair of opposed recessed portions or apertures 226A and 226B extending therethrough and which are shaped to receive respective ones of first prongs 224A and 224B. The prongs and apertures couple together in this non-limiting example when rearward barrier member 158.2 is positioned in a relatively forward position between ends 64.2 and 66.2 of pod 58.2 where cartridge-receiving arms 162A.2 and 162B.2 are positioned within recessed portions 218A and 218B of the pod. The rearward barrier member is received within and abuts a stop, in this example an annular interior shoulder 227 of pod 58.2 which inhibits any further forward movement thereof therepast. Rearward barrier member 158.2 includes in this non-limiting example one or more and in this example a pair of stops 228A and 228B best seen in FIG. 31C. The stops are adjacent apertures 226A and 226B and operatively couple to and are longitudinally spaced from elongate portions 208A and 208B of arms 162A.2 and 162B.2 in this non-limiting example. Stops 228A and 228B of rearward barrier member 158.2 abut shoulder 227 seen in FIG. 43D and are configured to in turn abut first prongs 224A and 224B. Outer hub 196.2 of needle assembly 74.2, which is coupled to cartridge 65.2, is thus inhibited (in this example via shoulder 227 and stops 228A and 228B so operatively connected to prongs 224A and 224B) from deployed or extending outwards of distal end 64.2 of pod 58.2 anew in the direction shown by arrow 229.

Cartridge-receiving arms 162A.2 and 162B.2 which are a part of and/or coupled to rearward barrier member 158.2, may thus be said to be configured inhibit deployment of the needle or needle assembly outwards in the second instance. Recessed portions 218A and 218B of pod 58.2 are also shaped so as to form catches 219A and 219B against which distal end portions 206A and 206B of the arms abut, inhibiting rearward axial movement of the arms thereby. The distal end portions of arms 162A.2 and 162B.2 also abut cartridge 65.2 (or barrel 66.2 thereof), thereby inhibiting radially inwards movement of the arms which may otherwise dislodge rearward barrier member 158.2 from its position axially. The arms so positioned/configured thus inhibit further retraction or extension of cartridge 65.2 and/or needle assembly 74.2 relative to pod 58.2.

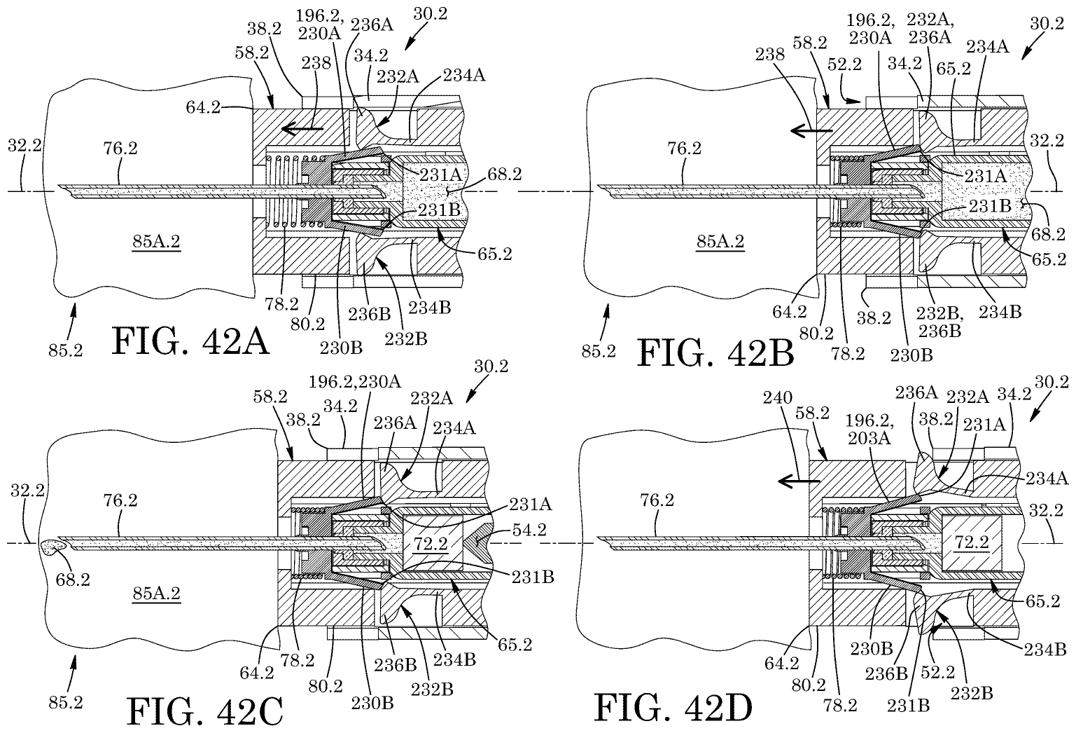

Referring back to FIGS. 29B and 29C, outer hub 196.2 of needle assembly 74.2 includes one or more radially outwardly-extending second elongate members or prongs 230A and 230B. As seen in FIG. 42C, the second prongs are configured to inhibit retraction of the needle or needle assembly while plunger 54.2 is causing medicament 68.2 to be outputted from cartridge 65.2. The following is a non-limiting embodiment which achieves this functionality.

Referring to FIGS. 29B and 29C, second prongs 230A and 230B are resilient and elongate. The second prongs are biased to extend radially outwards, with distal end portions 231A and 231B thereof seen in FIG. 29B most radially outwardly biased/positions in this example. Second prongs 230A and 230B are rectangular prisms in shape in this non-limiting embodiment; however, this is not strictly required. The second prongs extend towards proximal end 62.2 of pod 58.2 at least in part in this non-limiting embodiment as seen by prong 230A in FIG. 33B. Second prongs 230A and 230B are circumferentially spaced from first prongs 224A and 224B in this non-limiting example.

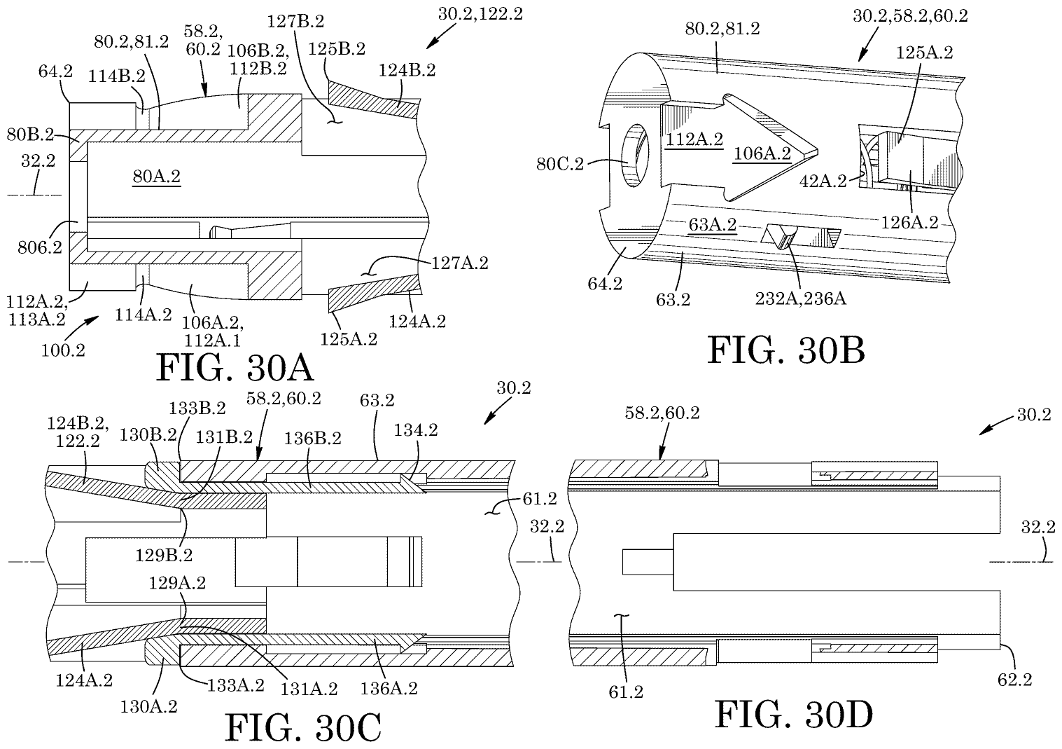

As seen in FIGS. 42A to 42D, pod 58.2 includes one or more and in this example a pair of opposed catch arms 232A and 232B. Each catch arm connects and is moveable radially inwards or outwards relative to forward end portion 80.2 of the pod via elongate members or portions 234A and 234B thereof in this non-limiting example. The elongate portions of the arms are resilient and bendable in this non-limiting example. Catch arms 232A and 232B include distal end portions 236A and 236B which are enlarged in this non-limiting example. The distal end portions of the catch arms connect to forward end portion 80.2 of pod 58.2 via elongate portions 234A and 234B of the catch arms: in this non-limiting example, the catch arms (including distal end portions and elongate portions thereof) are integrally connected to the forward end portion of the pod so as to form a unitary whole. As seen in FIG. 30B, each catch arm is configured to be biased radially outwards at least in part so as to extend outwards from pod housing 60.2, in this example extending outwards from exterior surface 63A.2 of wall 63.2 when pod 58.2 is free-of/removed-from the applicator. Insertion of pod 58.2 into applicator 34.2 seen in FIG. 35 causes catch arms 232A seen in FIG. 30B to abut wall 42.2 of outer body 40.2 of applicator 34 seen in FIG. 35 and bias radially inwards relative thereto.

Referring to FIGS. 42A and 42B, second prongs 230A and 230B so shaped/angled slidably extend past the catch arms when needle 76.2 is move to its deployed position in the first instance as shown by arrow 238. This process causes distal end portions 231A and 231B of the second prongs to bias radially inwards in part. Once needle 76.2 is fully deployed, the rearward-facing and radially outwardly-positioned distal end portions of second prongs 230A and 230B are configured to abut against the forward-facing distal end portions 236A and 236B of catch arms 232A and 232B and thus inhibit retraction of needle 76.2 and/or needle assembly 74.2 while the plunger is outputting medicament 68.2 from cartridge 65.2. Second prongs 230A and 230B and/or catch arms 232A and 232B may thus be referred to and/or comprise third coupling mechanism 167.2 which promotes positioning of needle 76.2 in the deployed position until medicament 68.2 has been outputted from pod 58.2, with removable of inward pressure on forward end portion 80.2 of the pod thereafter enabling the pod to be automatically ejected from applicator 34 as described in further detail below.

Once the medicament has been fully outputted and forward end portion 80.2 of pod 58.2 is no longer pressed against or abutting skin 85A.2 of person 85.2, compressed spring 57.2 seen in FIG. 41B is configured to push/bias the pod axially outwards from receptacle 52.2 of applicator 34.2. This causes catch arms 232A and 232B seen in FIG. 42D to extend out of the receptacle of the applicator 34.2 and bias radially outwards anew. Distal end portions 236A and 236B of the catch arms so positioned no longer abut second prongs 230A and 230B. Forward end portion 80.2 of pod 58.2 is thus free to move forwards relative to needle assembly 74.2 and needle 76.2, with spring 78.2 functioning to bias the forward end portion in a forward direction 240 relative to the needle assembly so as to move the pod towards to post-injection mode where the needle of the pod is covered/shielded seen in FIG. 43B.

The various mechanisms and parts herein described which enable outputting of medicament 68/68.1/68.2 from medicament injectors 30/30.1/30.2 may be referred to as medicament outputting means which automatically outputs medicament from pod 58/58.1/58.2 upon subsequent retraction of the pod relative to applicator 34/34.1/34.2 The various mechanisms and parts herein described which cause the pod to be automatically ejected from the applicator thereafter may be referred to as an ejection means which automatically ejects the pod from the applicator after outputting of the medicament from the pod or cartridge assembly.

It will be appreciated that many variations are possible within the scope of the invention described herein. Such variations may include but are not limited to one or more features described in U.S. patent application Ser. No. 16/006,732 filed in the United States Patent and Trademark Office on 12 Jun. 2018, U.S. patent application Ser. No. 16/800,804 filed in the United States Patent and Trademark Office on 25 Feb. 2020, and/or U.S. patent application Ser. No. 17/889,560 filed in the United States Patent and Trademark Office on 17 Aug. 2022, the disclosures of which are incorporated herein by reference.

Where a component (e.g. a member, assembly, device, etc.) is referred to herein, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A or B);
“approximately” when applied to a numerical value means the numerical value ±10%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and
“first” and “second” are used for descriptive purposes and cannot be understood as indicating or implying relative importance or indicating the number of indicated technical features.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, the exact numerical value ±5%, as well as all other numerical values that are near to or approximately equal to that numerical value. Unless otherwise indicated a particular numerical value is included in “about” a specifically recited numerical value where the particular numerical value provides the substantial equivalent of the specifically recited numerical value in the context in which the specifically recited numerical value is presented. For example, a statement that something has the numerical value of “about 10” is to be interpreted as: the set of statements:
in some embodiments the numerical value is 10;
in some embodiments the numerical value is in the range of 9.5 to 10.5;
and if from the context the person of ordinary skill in the art would understand that values within a certain range are substantially equivalent to 10 because the values with the range would be understood to provide substantially the same result as the value 10 then “about 10” also includes:
in some embodiments the numerical value is in the range of C to D where C and D are respectively lower and upper endpoints of the range that encompasses all of those values that provide a substantial equivalent to the value 10

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein has discrete components and features which may be readily separated from or combined with the features of any other described embodiment(s) without departing from the scope of the present invention.

Any aspects described above in reference to apparatus may also apply to methods and vice versa.

Any recited method can be carried out in the order of events recited or in any other order which is logically possible. For example, while processes or blocks are presented in a given order, alternative examples may perform routines having steps, or employ systems having blocks, in a different order, and some processes or blocks may be deleted, moved, added, subdivided, combined, and/or modified to provide alternatives or subcombinations. Each of these processes or blocks may be implemented in a variety of different ways. Also, while processes or blocks are at times shown as being performed in series, these processes or blocks may instead be performed in parallel, simultaneously or at different times.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible). This is the case even if features A and B are illustrated in different drawings and/or mentioned in different paragraphs, sections or sentences.

### ADDITIONAL DESCRIPTION

Examples of medicament injectors, including applicator and interchangeable pod with cartridges (and/or syringe and needle assembly) and pod cap assembly therefor, have been described. The following clauses are offered as further description.
(1) A medicament injector comprising: an applicator including a drive element; and an interchangeable pod or cartridge assembly loadable into the applicator so as to couple thereto, with subsequent retraction thereof actuating the drive element to engage the pod or cartridge assembly so as to output a medicament automatically therefrom, with the applicator being configured to thereafter automatically eject the pod or cartridge assembly.
(2) A medicament injector according to any clause herein, wherein loading of the pod or cartridge assembly into the applicator functions to prime the drive element and subsequent retraction of the pod or cartridge assembly into the applicator releases stored potential energy and causes the drive element to engage with the pod or cartridge assembly so as to output the medicament.
(3) A medicament injector according to any clause herein, wherein the energy required to manually insert the pod or cartridge assembly into the applicator is stored and subsequently released to automatically output the medicament from the cartridge and thereafter automatically eject the pod or cartridge assembly from the applicator.
(4) A medicament injector according to any clause herein, wherein the drive element comprises a spring which is movable from a primed or retracted position in a pre-injection mode, to an extended position in an injection mode.
(5) A medicament injector according to any clause herein, wherein the drive element comprises one or more elongate members which are movable from a primed or rearward position in a pre-injection mode, to a forward position in an injection mode.
(6) A medicament injector according to any clause herein, wherein the drive element comprises spring biased telescoping members which are movable from a primed or retracted position in a pre-injection mode, to an extended position in an injection mode.
(7) A medicament injector according to any clause herein, wherein the drive element is telescoping.
(8) A medicament injector according to any clause herein, wherein the applicator has a receptacle via which the pod or cartridge assembly is front-loaded into the applicator and through which the pod or cartridge assembly is ejected thereafter.
(9) A medicament injector according to any clause herein, wherein one or more of: the applicator is inhibited from contacting fluid medicament; the pod or cartridge assembly comprises all fluid paths of the medicament; and all medicament contacting and/or fluid path components thereof are automatically ejected from the receptacle thereafter following administration of the medicament.
(10) A medicament injector according to any clause herein, wherein the applicator is reusable.
(11) A medicament injector according to any clause herein, wherein upon the pod or cartridge assembly being ejected, the drive element is re-configured and/or configured to return to a native state to receive a subsequent said pod or cartridge assembly.
(12) A medicament injector comprising: an applicator; and an interchangeable pod or cartridge assembly shaped to receive a medicament-containing cartridge (and/or syringe and needle assembly) therewithin, the pod or cartridge assembly being resiliently biased to extend about a needle in a locked protected needle pre-injection mode, wherein the medicament is biased outwards from the needle when the pod or cartridge assembly is inserted into the applicator and the pod or cartridge assembly is moved from the locked protected needle pre-injection mode to a partially retracted mode, and wherein the pod or cartridge assembly is configured to be automatically ejected from the receptacle thereafter following administration of the medicament.
(13) A medicament injector according to any clause herein, wherein the applicator includes a drive element which biases the medicament outwards from the needle.
(14) A medicament injector according to any clause herein, wherein at least a front end portion of the pod or cartridge assembly is spring biased outwards from the applicator and configured selectively retract at least in part within the applicator to actuate release of the medicament.
(15) A medicament injector comprising: an applicator; an interchangeable pod or cartridge assembly including or being shaped to receive a medicament-containing cartridge (and/or syringe and needle assembly); and a barrier member coupled to the pod or cartridge assembly, the barrier member being configured such that removal thereof to enable access to the medicament requires the pod or cartridge assembly to be inserted within and/or coupled to the applicator.
(16) A medicament injector according to any clause herein, wherein the medicament-containing cartridge is user-insertable.
(17) A medicament injector according to any clause herein, wherein the medicament-containing cartridge is a third party product.
(18) A medicament injector according to any clause herein, including a needle via which the medicament is injectable.
(19) A medicament injector according to any clause herein, wherein the barrier member is configured to enable fluid communication between the needle and the medicament upon the pod or cartridge assembly being inserted within, coupled to and/or snap-fit connected to the applicator.
(20) A medicament injector according to any clause herein, wherein the barrier member is configured to interference fit couple to an end portion of the pod or cartridge assembly.
(21) A medicament injector according to any clause herein, wherein the barrier member is configured to inhibit access to the medicament until the pod or cartridge assembly is inserted within, coupled to and/or snap-fit connected to the applicator.
(22) A medicament injector comprising: an applicator; and an interchangeable pod or cartridge assembly including or being shaped to receive a medicament-containing cartridge (and/or syringe and needle assembly), a barrier member coupled to the pod or cartridge assembly and a locking mechanism that inhibits removal of the barrier member until the pod or cartridge assembly is inserted within, coupled to and/or snap-fit connected to the applicator.
(23) A medicament injector according to any clause herein, wherein the applicator includes a receptacle and wherein the locking mechanism inhibits removal of the barrier member until the pod or cartridge assembly is inserted within the receptacle of the applicator.
(24) A medicament injector according to any clause herein, wherein the barrier member is reversible.
(25) A medicament injector according to any clause herein, wherein the barrier member couples to a needle end of the applicator.
(26) A medicament injector according to any clause herein, wherein the pod or cartridge assembly has an interior and wherein the barrier member is configured to inhibit access to the interior of the pod or cartridge assembly and/or render the medicament inaccessible until the pod or cartridge assembly is inserted into, coupled to and/or snap-fit connected to the applicator.
(27) A medicament injector according to any clause herein, wherein the pod or cartridge assembly being received by and/or coupling to the applicator functions to automatically release the barrier member from the pod or cartridge assembly.
(28) A medicament injector according to any clause herein, wherein the locking mechanism has a locked mode in which axial and rotational movement of the barrier member relative to the pod or cartridge assembly is inhibited and wherein the locking mechanism has an unlocked mode in which the barrier member is movable axially outwards relative to the pod or cartridge assembly so as to remove the barrier member from the pod or cartridge assembly.
(29) A medicament injector according to any clause herein, wherein the barrier member is configured to enable fluid communication between the needle and the medicament upon the locking mechanism moving to the unlocked mode thereof.
(30) A medicament injector according to any clause herein, wherein the barrier member couples to the pod or cartridge assembly via a male member and a female member shaped to receive the male member, and wherein insertion of the pod or cartridge assembly into the applicator causes the male member to dislodge from the female member at least in part.
(31) A medicament injector according to any clause herein, wherein the female member includes a catch and wherein the male member is dislodged from the catch upon the pod or cartridge assembly being inserted into the applicator.
(32) A medicament injector according to any clause herein, wherein the applicator includes an actuating or deflecting member shaped to dislodge the male member from the female member upon the pod or cartridge assembly being inserted into, coupling to and/or snap fit connecting with the applicator.
(33) A medicament injector according to any clause herein, wherein the deflecting member comprises a recessed portion of the applicator.
(34) A medicament injector according to any clause herein, wherein the recessed portion of the applicator is slot-shaped.
(35) A medicament injector according to any clause herein, wherein the male member comprises one or more resilient elongate protrusions.
(36) A medicament injector according to any clause herein, wherein the medicament injector has a longitudinal axis, wherein the male member which couples to the barrier member to the pod or cartridge assembly, extends longitudinally, laterally and radially in part when in the locked position and wherein said male member when in the unlocked position extends more or less longitudinally and/or more or less laterally and/or more or less radially compared to when in said locked position.
(37) A medicament injector according to any clause herein, wherein the male member is V-shaped.
(38) A medicament injector according to any clause herein, wherein the male member is hook or harpoon-shaped.
(39) A medicament injector according to any clause herein, wherein the male member comprises a pair of resilient, angled elongate protrusions.
(40) A medicament injector according to any clause herein, wherein the angled elongate protrusions are angularly spaced in the locked position and are biased towards each other in the unlocked position.
(41) A medicament injector according to any clause herein, wherein the female member comprises a pair of opposed or mirrored catches.
(42) A medicament injector according to any clause herein, wherein the female member is triangular at least in part.
(43) A medicament injector according to any clause herein, wherein the female member comprises a first recessed portion that facilitates passage of the male member therethrough and a second recessed portion in fluid communication and/or smooth and/or continuous connection with the first recessed portion, with the second recessed portion being shaped to inhibit removal of the male member therefrom.
(44) A medicament injector according to any clause herein, wherein the female member comprises a first recessed portion that is rectangular in side profile and a second recessed portion in fluid communication and/or smooth and/or continuous connection with the first recessed portion, with the second recessed portion being triangular in side profile.
(45) A medicament injector according to any clause herein, wherein the pod or cartridge assembly being inserted into and/or coupling with the applicator functions to deform the male member.
(46) A medicament injector according to any clause herein, wherein the barrier member is locked in place until the pod or cartridge assembly is inserted into and/or couples with the applicator.
(47) A medicament injector according to any clause herein, wherein the barrier member is snap-fit in place until the pod or cartridge assembly is inserted into the applicator.
(48) A medicament injector according to any clause herein, wherein the barrier member is removable only upon the pod or cartridge assembly being substantially fully inserted into the applicator and/or wherein the barrier member is configured to enable fluid communication between the needle and the medicament upon the pod or cartridge assembly being substantially fully inserted into the applicator.
(49) A medicament injector according to any clause herein, wherein the barrier member comprises one or more of: a pod cap assembly, a cartridge assembly cap, an outer cap and an inner cap.
(50) A medicament injector according to any clause herein, wherein the barrier member comprises an outer cap and an inner cap telescopically coupled to the outer cap thereof.
(51) A medicament injector comprising: an applicator; an interchangeable pod or cartridge assembly having a needle end and including or being shaped to receive a medicament-containing cartridge (and/or syringe and needle assembly); and a barrier member configured to enclose the needle end of the pod or cartridge assembly and inhibit access therethrough until the pod or cartridge assembly is inserted within, coupled to and/or snap fit connected to the applicator.
(52) A medicament injector comprising: an applicator; an interchangeable pod or cartridge assembly including and/or shaped to receive a medicament-containing cartridge (and/or syringe and needle assembly); and a reversible barrier member configured to inhibit access to the pod or cartridge assembly until the pod or cartridge assembly is inserted within, coupled to and/or snap-fit connected to the applicator.
(53) A medicament injector according to any clause herein, wherein the barrier member couples to the pod or cartridge assembly so as to inhibit access to the needle assembly.
(54) A medicament injector according to any clause herein, wherein the barrier member is telescoping.
(55) A medicament injector according to any clause herein, wherein the needle assembly is telescoping.
(56) A medicament injector according to any clause herein, wherein the needle assembly includes an outer hub and an inner hub slidable relative to the outer hub thereof.
(57) A medicament injector according to any clause herein, wherein the outer hub of the needle assembly is shaped to receive the inner hub of the needle assembly upon the pod or cartridge assembly being inserted within, coupling to and/or snap fit connecting with the applicator.
(58) A medicament injector according to any clause herein, wherein the needle assembly is configured to inhibit communication between the needle thereof and the medicament until the pod or cartridge assembly is inserted within, coupled to and/or snap fit connected with the applicator.
(59) A medicament injector according to any clause herein, wherein the pod or cartridge assembly includes a needle sheath or sleeve retraction of which is inhibited when the barrier member is coupled to the pod or cartridge assembly.
(60) A medicament injector according to any clause herein, including a needle protection assembly configured to extend about a needle in a pre-injection mode and expose the needle in an injection mode, with the barrier member being configured to inhibit positioning of the needle protection assembly in the injection mode when the barrier member is coupled to the pod or cartridge assembly.
(61) A medicament injector according to any clause herein, wherein the barrier member is shaped to promote sterility of the pod or cartridge assembly and/or interior thereof.
(62) A medicament injector according to any clause herein, wherein the barrier member is shaped to extend about the needle in an airtight manner.
(63) A medicament injector according to any clause herein, wherein the barrier member includes an inner sheath or sleeve shaped to receive and extend about the needle.
(64) A medicament injector according to any clause herein, wherein the pod or cartridge assembly includes and/or is shaped to receive therewithin a cartridge (and/or syringe and needle assembly).
(65) A medicament injector according to any clause herein, wherein the inner sheath or sleeve is shaped to abut a neck of the cartridge (and/or syringe and needle assembly) when the barrier member couples to the pod or cartridge assembly.
(66) A medicament injector according to any clause herein, wherein the barrier member is shaped to inhibit inadvertent deployment of the needle when the pod or cartridge assembly is being inserted into the applicator.
(67) A medicament injector according to any clause herein, wherein the applicator has a front end through which the pod or cartridge assembly is received.
(68) A medicament injector according to any clause herein, wherein the medicament injector is a front-loaded said medicament injector.
(69) A medicament injector comprising: an applicator; an interchangeable pod or cartridge assembly including or being shaped to include a medicament-containing cartridge (and/or syringe and needle assembly); and a safeguard mechanism coupled to the pod or cartridge assembly, the safeguard mechanism configured to enable axial movement of the pod or cartridge assembly relative to the applicator during an injection mode and upon the pod or cartridge assembly being removed from the applicator thereafter, being configured to inhibit reinsertion of the pod or cartridge assembly into the applicator once more.
(70) A medicament injector according to any clause herein, wherein the applicator includes one or more tabs positioned to engage with passageways of a housing of the pod or cartridge assembly when the pod or cartridge assembly is inserted into the applicator.
(71) A medicament injector according to any clause herein, wherein the one or more tabs positioned to facilitate insertion of the pod or cartridge assembly within the applicator in an aligned manner.
(72) A medicament injector according to any clause herein, where the one or more tabs are configured to ensure that the needle remains in an injection position while the medication is being injected.
(73) A medicament injector according to any clause herein, wherein the safeguard mechanism is configured to inhibit access to and/or pushing on a rear of the cartridge (and/or syringe and needle assembly).
(74) A medicament injector according to any clause herein, wherein the pod or cartridge includes a rearward barrier member which restricts access to the cartridge from a rear or proximal end thereof.
(75) A medicament injector according to any clause herein, wherein the pod or cartridge assembly has a proximal end and a distal end spaced-apart from the proximal end thereof, and wherein the rearward barrier member is moveable axially towards the distal end of the pod or cartridge assembly during the injection mode and thereafter moveable axially towards the proximal end of the pod or cartridge assembly, wherein upon the pod or cartridge assembly being removed from the applicator, subsequent movement of the rearward barrier member towards the distal end of the pod or cartridge assembly is inhibited.
(76) A medicament injector according to any clause herein, wherein the rearward barrier member is biased towards the distal end of the pod or cartridge assembly.
(77) A medicament injector according to any clause herein, wherein the rearward barrier member comprises at least one protrusion and wherein the pod or cartridge assembly includes a housing with an axially-extending passageway shaped to receive the protrusion.
(78) A medicament injector according to any clause herein, wherein the passageway includes an inner portion along which the protrusion moves in the injection mode and wherein the passageway includes an outer portion along which the protrusion moves thereafter.
(79) A medicament injector according to any clause herein, including a catch positioned along the passageway so as to inhibit movement of the protrusion upon the pod or cartridge assembly being removed from the applicator.
(80) A medicament injector according to any clause herein, wherein the rearward barrier member comprises a housing and a pair of protrusions positioned therewithin, with the protrusions being moveable along passageways of the pod or cartridge assembly towards the distal end of the pod or cartridge assembly in the injection mode to enable passage of the drive element, with the protrusions being biased towards the proximal end of the pod or cartridge assembly thereafter as the pod or cartridge assembly is removed from the applicator, and with the protrusions being configured to lock in place thereafter.
(81) A medicament injector according to any clause herein, wherein each said passageway includes a portion which inhibits movement of its corresponding protrusion therefrom.
(82) A medicament injector according to any clause herein, wherein the protrusions are spaced to inhibit passage of the drive element therethrough until the needle is fully extended.
(83) A medicament injector according to any clause herein, wherein the protrusions are moveable radially outwards upon the needle being fully extended so as to thereafter enable the drive element therethrough.
(84) A medicament injector according to any clause herein, wherein the rearward barrier member comprises a housing and a pair of protrusions positioned therewithin, with the protrusions being moveable along passageways of the pod or cartridge assembly towards the distal end of the pod or cartridge assembly, with the protrusions being moveable radially outwards to enable the drive element of the applicator therethrough when the needle is in a fully extending position.
(85) A medicament injector according to any clause herein, wherein the safeguard mechanism includes one or more latch members which extend radially outwards, bias radially inwards upon the pod or cartridge assembly being received by the applicator in a first instance and, upon the pod or cartridge assembly subsequently being removed from the applicator, are configured to inhibit re-insertion of the cartridge (and/or syringe and needle assembly) into the applicator once more.
(86) A medicament injector according to any clause herein, wherein the safeguard mechanism includes a sleeve which extends about the cartridge (and/or syringe and needle assembly) and one or more latch members which bias the sleeve to hold the cartridge (and/or syringe and needle assembly) in place in a pre-injection mode.
(87) A medicament injector according to any clause herein, wherein the sleeve has an outer surface that is curved and/or a first of outwardly concave and convex and wherein each said latch member of the safeguard mechanism has an inner surface that is curved and/or a second of outwardly concave and convex and/or shaped to mirror the shape of the outer surface of the sleeve.
(88) A medicament injector according to any clause herein, wherein the sleeve is positioned to align with the one or more latch members of the safeguard mechanism in the pre-injection mode.
(89) A medicament injector according to any clause herein, wherein the sleeve is axially spaced from the one or more latch members of the safeguard mechanism in the injection and post-injection modes.
(90) A medicament injector according to any clause herein, wherein the rearward barrier member is positioned to inhibit the one or more latch members of the safeguard mechanism from being radially biased after the pod or cartridge assembly has been removed from the applicator.
(91) A medicament injector comprising: an applicator; an interchangeable pod or cartridge assembly shaped to be insertable within the applicator, the pod or cartridge assembly including a cartridge (and/or syringe and needle assembly) shaped to receive or contain a medicament therewithin; and a safeguard mechanism comprising a sleeve which extends about the cartridge (and/or syringe and needle assembly) and one or more latch members which bias and/or compress the sleeve to hold the cartridge (and/or syringe and needle assembly) in place in a pre-injection mode, with the one or more latch members being configured to bias outwards and/or reduce compression of the sleeve in an injection mode and/or post-injection mode.
(92) A medicament injector comprising: an applicator; an interchangeable pod or cartridge assembly shaped to be insertable within the applicator, the pod or cartridge assembly including a cartridge (and/or syringe and needle assembly) shaped to receive or contain a medicament therewithin; and a guiding mechanism configured to enable the cartridge (and/or syringe and needle assembly) to move axially during an injection mode and configured to lock in place adjacent a proximal end of the pod or cartridge assembly upon the pod or cartridge assembly being removed from the applicator thereafter.
(93) A medicament injector according to any clause herein, including a first coupling mechanism that inhibits axial movement of the pod or cartridge assembly relative to the applicator.
(94) A medicament injector according to any clause herein, wherein the first coupling mechanism comprises one or more latch members.
(95) A medicament injector according to any clause herein, wherein each said latch member of the first coupling mechanism is configured to bias radially inwards in the injection mode.
(96) A medicament injector according to any clause herein, wherein the pod or cartridge assembly comprises the one or more latch members of the first coupling mechanism and wherein the first coupling mechanism comprise one or more recessed portions shaped to receive the one or more latch members of the first coupling mechanism.
(97) A medicament injector according to any clause herein, wherein the applicator comprising the one or more recessed portion of the first coupling mechanism.
(98) A medicament injector according to any clause herein, including a second coupling mechanism that inhibits axial movement of the cartridge (and/or syringe and needle assembly) relative to the pod or cartridge assembly.
(99) A medicament injector according to any clause herein, wherein the first and second coupling mechanisms are axially spaced-apart.
(100) A medicament injector according to any clause herein, wherein the second coupling mechanism comprises one or more latch members.
(101) A medicament injector according to any clause herein, wherein the second coupling mechanism comprises a sleeve biased and/or compressed via the one or more latch members thereof in the pre-injection mode.
(102) A medicament injector according to any clause herein, wherein the one or more latch members of the second coupling mechanism are configured to bias radially outwards in the injection mode.
(103) A medicament injector according to any clause herein, wherein the pod or cartridge assembly comprises the one or more latch members of the second coupling mechanism and wherein the second coupling mechanism comprise one or more recessed portions shaped to receive the one or more latch members of the second coupling mechanism.
(104) A medicament injector according to any clause herein, wherein the applicator comprising the one or more recessed portion of the second coupling mechanism.
(105) A medicament injector comprising: an applicator with at least one first recessed portion and at least one second recessed portion axially spaced from the at least one first recessed portion thereof; and an interchangeable pod or cartridge assembly including or being shaped to receive a medicament-containing cartridge (and/or syringe and needle assembly), with the pod or cartridge assembly including at least one first latch member shaped to fit within the at least one first recessed portion and inhibit axial movement of the pod or cartridge assembly relative to the applicator in a pre-injection mode, and with the pod or cartridge assembly including at least one second latch member shaped to inhibit axial movement of the cartridge (and/or syringe and needle assembly) relative to the pod or cartridge assembly in the pre-injection mode, with the at least one second latch member being configured to fit at least in part within the at least one second recessed portion so as to enable axial movement of the cartridge (and/or syringe and needle assembly) in an injection mode.
(106) A medicament injector according to any clause herein, wherein each said latch member is elongate and resilient.
(107) A medicament injector according to any clause herein, wherein each said latch member is configured to bias radially outwards.
(108) A medicament injector according to any clause herein, wherein the at least one first latch member is configured to bias radially inwards during the injection mode and/or wherein the at least one second latch member is configured to bias radially outwards during the injection mode.
(109) A medicament injector according to any clause herein, wherein the at least one first latch member is axially spaced from the at least one second latch member.
(110) A medicament injector according to any clause herein, wherein the at least one first recessed portion of the applicator comprises an aperture extending through an outer wall of the applicator.
(111) A medicament injector according to any clause herein, wherein the at least one second recessed portion of the applicator extends from an inner surface towards an outer surface of the outer wall of the applicator.
(112) A medicament injector according to any clause herein, wherein the barrier member includes an outer portion and an inner portion slidably coupled to and received within the outer portion thereof.
(113) A medicament injector according to any clause herein, wherein one of the inner and outer portion of the barrier member is configured to inhibit movement of the other of the inner and outer portion of the barrier member until the pod or cartridge assembly is inserted within, coupled to and/or snap fit connected with the applicator.
(114) A medicament injector according to any clause herein, wherein the inner portion of the barrier member is moveable from an extended or locked position, to a retracted or unlocked position upon the pod or cartridge assembly being inserted within, coupling to and/or snap fit connecting with the applicator.
(115) A medicament injector according to any clause herein, wherein movement of the inner portion of the barrier member from the extended or locked position thereof to the retracted or unlocked position thereof, enables the needle to pierce the cartridge so as to be in fluid communication with the medicament.
(116) A medicament injector according to any clause herein, wherein the inner portion of the barrier member abuts the cartridge in the extended or locked position thereof.
(117) A medicament injector according to any clause herein, wherein the inner portion of the barrier member functions as an end stop for the cartridge so user-inserted, thereby inhibiting the cartridge from moving longitudinally therepast.
(118) A medicament injector according to any clause herein, wherein the inner portion of the barrier member extends past and about an inner needle tip of the needle assembly.
(119) A medicament injector according to any clause herein, wherein the barrier member is shaped to inhibit premature puncturing of the cartridge when the cartridge is user-inserted into the pod or cartridge assembly.
(120) A medicament injector according to any clause herein, wherein the needle assembly (and/or an inner hub thereof) is shaped to receive, couple with and/or snap fit connect with the cartridge so user-inserted into the pod or cartridge assembly.
(121) A medicament injector according to any clause herein, wherein the needle assembly (and/or the inner hub thereof) includes a catch shaped to extend about and couple to the cartridge so user-inserted into the pod or cartridge assembly so as to inhibit removal of the cartridge thereafter.
(122) A medicament injector according to any clause herein, wherein the pod or cartridge assembly is configured to make an audible sound or click to indicate when the cartridge so user-inserted is properly locked into place.
(123) A medicament injector according to any clause herein, wherein the needle assembly (and/or an outer hub thereof) includes one or more radially outwardly-extending first prongs configured to enable deployment of the needle or needle assembly outwards from a distal or needle end of the pod or cartridge assembly in a first instance and upon the needle or needle assembly moving back to a covered or retracted position, inhibit deployment of the needle or needle assembly outwards therefrom thereafter.
(124) A medicament injector according to any clause herein, wherein the one or more first prongs extend towards the distal or needle end of the pod or cartridge assembly at least in part.
(125) A medicament injector according to any clause herein, wherein the needle assembly (and/or an outer hub thereof) includes one or more radially outwardly-extending second prongs configured to inhibit retraction of the needle or needle assembly while the drive element is outputting the medicament from the cartridge.
(126) A medicament injector according to any clause herein, wherein the one or more second prongs extend towards the proximal end of the pod or cartridge assembly at least in part.
(127) A medicament injector according to any clause herein, wherein the pod or cartridge assembly includes one or more latch members which engage with the one or more second prongs so as to inhibit retraction of the needle or needle assembly while the drive element is outputting the medicament from the cartridge.
(128) A medicament injector according to any clause herein, wherein the pod or cartridge assembly includes one or more latch members configured to receive the cartridge therethrough.
(129) A medicament injector according to any clause herein, wherein the one or more latch members configured to receive the cartridge therethrough, are radially outwardly-biased.
(130) A medicament injector according to any clause herein, wherein the pod or cartridge assembly includes one or more deflecting members which biases the one or more latch members configured to receive the cartridge therethrough, radially inwards so as to inhibit access to the cartridge when the pod or cartridge assembly is inserted into the applicator.
(131) A medicament injector according to any clause herein, wherein the one or more latch members configured to receive the cartridge therethrough, are configured to inhibit the drive element from engaging the cartridge until the needle or needle assembly is moved to a deployed position.
(132) A medicament injector according to any clause herein, wherein the one or more latch members configured to receive the cartridge therethrough, are configured to move radially outwards so as to enable the drive element to extend therepast in the deployed position of the needle or needle assembly.
(133) A medicament injector according to any clause herein, wherein the one or more latch members configured to receive the cartridge therethrough, are received within radially outwardly extending recessed portions of the pod or cartridge assembly in the deployed position of the needle or needle assembly.
(134) A medicament injector according to any clause herein, wherein each said latch member configured to receive the cartridge therethrough, includes a distal end portion which is enlarged and/or bulbous.
(135) A medicament injector according to any clause herein, wherein the one or more latch members configured to receive the cartridge therethrough, are configured to enable deployment of the needle or needle assembly outwards from a distal or needle end of a housing of the pod or cartridge assembly in a first instance and upon the needle or needle assembly moving back to a covered or retracted position, inhibit deployment of the needle or needle assembly outwards therefrom thereafter.
(136) A medicament injector according to any clause herein, wherein the inner portion of the barrier member comprises the one or more male members of the barrier member, wherein the outer portion of the barrier member has a first one or more channels via which the one or more male members are received, with one or more catches adjacent the first one or more channels inhibiting retraction of the inner portion and/or one or more male members of the barrier member relative to the outer portion of the barrier member and/or inhibiting movement of the outer portion relative to the needle assembly, until the pod or cartridge assembly is inserted within, couples to and/or snap fit connects with the applicator.
(137) A medicament injector according to any clause herein, wherein the inner portion of the barrier member includes a second one or more male members or catches and wherein outer portion has a second one or more channels shaped to engage with the second one or more male members or catches such that removal of the outer portion of the barrier member promotes and/or facilitates removal of the inner portion of the barrier member.
(138) A medicament injector according to any clause herein, wherein the barrier member comprises a pod cap and a locking member.
(139) A medicament injector comprising: an interchangeable pod or cartridge assembly including a needle assembly, including a first telescoping mechanism and including or being shaped to receive a medicament-containing cartridge (and/or syringe and needle assembly); and an applicator shaped to receive the pod or cartridge assembly and actuate the first telescoping mechanism thereby to cause the needle assembly to pierce the cartridge, the applicator including a second telescoping mechanism actuation of which thereafter promotes outputting of the medicament from the needle.
(140) A medicament injector comprising: an applicator including a drive element; an interchangeable pod or cartridge assembly has a needle end and an insertion end shaped to receive a user-insertable cartridge therein, the pod or cartridge assembly being loadable into the applicator so as to couple thereto and prime the drive element, with further retraction thereof actuating/releasing the drive element to engage the pod or cartridge assembly so as to output a medicament automatically therefrom.
(141) A medicament injector according to any clause herein, wherein the applicator is configured to automatically eject the pod or cartridge assembly from the applicator after outputting of the medicament from the pod or cartridge assembly.
(142) A medicament injector comprising: an applicator; an interchangeable pod or cartridge assembly loadable into the applicator so as to couple thereto; a medicament outputting means which automatically outputs a medicament from the pod or cartridge assembly upon subsequent retraction of the pod or cartridge relative to the applicator; and an ejection means which automatically ejects the pod or cartridge assembly from the applicator after outputting of the medicament from the pod or cartridge assembly.
(143) A medicament injector according to any clause herein, wherein the medicament injector includes: a first coupling mechanism via which the pod or cartridge assembly and the applicator couple together, the first coupling mechanism inhibiting axial movement of the pod or cartridge assembly outwardly of the applicator in a first instance and enabling retraction of a forward end portion of the pod or cartridge assembly relative to the applicator, which decouples the first coupling mechanism and initiates an automatic injection process; a second coupling mechanism that inhibits axial movement of the cartridge (and/or syringe and needle assembly) relative to the pod or cartridge assembly and decouples upon initiation of the automatic injection process, to enable the needle assembly to move outwards of the pod or cartridge assembly to a deployed position via which the medicament is outputted therefrom; and/or a third coupling mechanism which promotes positioning of the needle assembly in the deployed position until the medicament has been outputted from the cartridge (and/or syringe and needle assembly), with removable of inward pressure on the forward end portion of the pod or cartridge assembly thereafter enabling the pod or cartridge assembly to be automatically ejected from the applicator.
(144) A medicament injector comprising: an applicator; an interchangeable pod or cartridge assembly including a cartridge (and/or syringe and needle assembly) shaped to receive or contain a medicament therewithin; and a pod cap assembly coupled to and configured to be removable from the pod or cartridge assembly upon the pod or cartridge assembly being inserted within the applicator.
(145) A medicament injector comprising: an applicator; an interchangeable pod or cartridge assembly including a cartridge (and/or syringe and needle assembly) shaped to receive or contain a medicament therewithin and having a needle end; and a barrier member configured to enclose the needle end of the pod or cartridge assembly and inhibit access therethrough until the pod or cartridge assembly is inserted within the applicator.
(146) A medicament injector comprising: an applicator; an interchangeable pod or cartridge assembly including a cartridge (and/or syringe and needle assembly) shaped to receive or contain a medicament therewithin and having a needle end; and a barrier member configured to inhibit access to the pod or cartridge assembly via the needle end until the pod or cartridge assembly is inserted within the applicator.
(147) A medicament injector comprising: an applicator; and an interchangeable pod or cartridge assembly including a cartridge (and/or syringe and needle assembly) shaped to receive or contain a medicament therewithin and including a reversible barrier member coupled to the body thereof, the reversible barrier member being configured to inhibit access to a needle until the pod or cartridge assembly being inserted within the applicator.
(148) A medicament injector comprising: an applicator; and an interchangeable pod or cartridge assembly, the pod or cartridge assembly including a cartridge (and/or syringe and needle assembly) shaped to receive or contain a medicament therewithin and including a reversible barrier member coupled to the body thereof, the reversible barrier member being configured to inhibit access to the pod or cartridge assembly until the pod or cartridge assembly is inserted within the applicator.
(149) A medicament injector comprising: an applicator; an interchangeable pod or cartridge assembly including and/or operatively connected to a needle assembly; and a reversible barrier member coupled to the pod or cartridge assembly, the reversible barrier member being configured to inhibit access to the needle assembly until the pod or cartridge assembly is inserted within the applicator.
(150) A medicament injector comprising: an applicator; an interchangeable pod or cartridge assembly with a cartridge (and/or syringe and needle assembly) shaped to receive or contain a medicament therewithin; and a barrier member coupled to the body, the barrier member being configured such that removal thereof to enable access to the medicament requires the pod or cartridge assembly to be inserted within the applicator and couple to the applicator, with the barrier member being configured to render the medicament inaccessible when the pod or cartridge assembly is not inserted into the applicator.
(151) A medicament injector according to any clause herein, wherein the drive element barrier member comprises a housing and protrusion(s) positioned therewithin, with the protrusion(s) being moveable along passageways of the pod or cartridge assembly towards the distal end of the pod or cartridge assembly in the injection mode to enable passage of the drive element, with the protrusion(s) being biased towards the proximal end of the pod or cartridge assembly thereafter as the pod or cartridge assembly is removed from the applicator, and with the protrusion(s) being configured to lock in place thereafter to inhibit subsequent passage of the drive element and/or deployment of the needle.
(152) A medicament injector according to any clause herein, wherein each said passageway includes portion(s) which inhibits movement of its corresponding protrusion therefrom.
(153) A medicament injector according to any clause herein, wherein the protrusions are spaced to inhibit passage of the drive element therethrough until the needle is fully extended.
(154) A medicament injector according to any clause herein, wherein the protrusions are moveable radially outwards upon the needle being fully extended so as to thereafter enable the drive element therethrough.
(155) A medicament injector according to any clause herein, wherein each said passageway includes a zig-zag portion which inhibits movement of its corresponding protrusion therefrom.
(156) A medicament injector according to any clause herein, wherein the drive element barrier member comprises a housing and a pair of protrusions positioned therewithin, with the protrusions being moveable along passageways of the pod or cartridge assembly towards the distal end of the pod or cartridge assembly, with the protrusions being moveable radially outwards to enable the drive element of the applicator therethrough when the needle is in a fully extending position.
(157) A medicament injector comprising: an applicator; an interchangeable pod or cartridge assembly shaped to be insertable within the applicator, the pod being configured to receive or include a cartridge (and/or syringe and needle assembly) shaped to receive or contain a medicament therewithin; and a drive element comprising two or more telescoping members which are movable from a retracted or nested position in a pre-injection mode, to an extended position in an injection mode.
(158) A medicament injector comprising: an applicator having a length extending between proximal and distal ends thereof; an interchangeable pod or cartridge assembly shaped to be insertable within the applicator, the pod or cartridge assembly being configured to receive or include a cartridge (and/or syringe and needle assembly) shaped to receive or contain a medicament therewithin; and a drive element comprising telescoping members which are movable from a retracted or nested position in a pre-injection mode, to an extended position in an injection mode.
(159) A medicament injector according to any clause herein, wherein the drive element is configured to extend within the cartridge (and/or syringe and needle assembly) in the extended position thereof.
(160) A medicament injector comprising: an applicator system including a receptacle; and an interchangeable pod having an injection end, having an insertion end via which the pod is insertable into the applicator system and including a cartridge (and/or syringe and needle assembly) shaped to receive or contain a medicament therewithin, wherein the cartridge (and/or syringe and needle assembly) is positioned towards or closer to the insertion end when the pod is inserted into the applicator system and wherein the cartridge (and/or syringe and needle assembly) is moveable and/or positioned towards or closer to the injection end when the medicament injector is an injection mode.
(161) A medicament injector according to any clause herein, wherein the cartridge (and/or syringe and needle assembly) is configured to be moveable and/or revert back towards or closer to the insertion end upon the pod being removed from the applicator system.
(162) A medicament injector according to any clause herein, wherein the cartridge (and/or syringe and needle assembly) includes a needle that is extendable outwards from the injection end of the pod during the injection mode.
(163) A medicament injector according to any clause herein, wherein the cartridge (and/or syringe and needle assembly) is shaped to receive or contain medicament therewithin and a needle coupled to the cartridge or barrel and in fluid communication with the medicament.
(164) A medicament injector according to any clause herein, wherein the needle is axially rearwardly spaced from the injection end of the pod in the pre-injection and post-injection modes and wherein the cartridge or barrel is axially forwardly spaced from the insertion end of the pod in the pre-injection and post-injection modes.
(165) A barrier member according to any clause herein.
(166) An interchangeable pod or cartridge assembly according to any clause herein.
(167) In combination, an interchangeable pod or cartridge assembly according to any clause herein together with a barrier member according to any clause herein.
(168) A pod cap assembly according to any clause herein.
(169) Apparatus including any new and inventive feature, combination of features, or sub-combination of features as described herein.
(170) Methods including any new and inventive steps, acts, combination of steps and/or acts or sub-combination of steps and/or acts as described herein.

It is therefore intended that the following appended claims and claims hereafter introduced are interpreted to include all such modifications, permutations, additions, omissions, and subcombinations as may reasonably be inferred. The scope of the claims should not be limited by the preferred embodiments set forth in the examples, but should be given the broadest interpretation consistent with the description as a whole.

What is claimed is:

1. A medicament injector comprising:
an applicator including a drive element; and
an interchangeable pod or cartridge assembly loadable into the applicator so as to couple thereto, with subsequent retraction thereof actuating the drive element to engage the pod or cartridge assembly so as to output a medicament automatically therefrom, with the applicator being configured to thereafter automatically eject the pod or cartridge assembly.

2. A medicament injector according to any claim 1, wherein loading of the pod or cartridge assembly into the applicator functions to prime the drive element and/or store potential energy, and subsequent retraction of the pod or cartridge assembly into the applicator releases the stored potential energy and causes the drive element to engage with the pod or cartridge assembly so as to output the medicament.

3. A medicament injector according to claim 1, wherein the energy required to manually insert the pod or cartridge assembly into the applicator is stored and subsequently released to automatically output the medicament from the pod or cartridge assembly and thereafter automatically eject the pod or cartridge assembly from the applicator.

4. A medicament injector according to claim 1, wherein the drive element comprises spring biased telescoping members which are movable from a primed or retracted position in a pre-injection mode, to an extended position in an injection mode.

5. A medicament injector according to claim 1, wherein the applicator has a receptacle via which the pod or cartridge assembly is front-loaded into the applicator and through which the pod or cartridge assembly is ejected thereafter.

6. A medicament injector according to claim 1, wherein one or more of: the applicator is inhibited from contacting fluid medicament; the pod or cartridge assembly comprises all fluid paths of the medicament; and all medicament contacting and/or fluid path components thereof are automatically ejected from the applicator thereafter following administration of the medicament.

7. A medicament injector according to claim 1, wherein upon the pod or cartridge assembly being ejected, the applicator is reusable with the drive element thereof being re-configured and/or configured to return to a native state to receive a subsequent said pod or cartridge assembly.

8. A medicament injector according to claim 1, wherein the pod or cartridge assembly includes a medicament-containing cartridge (and/or syringe and needle assembly) therewithin or wherein the pod or cartridge assembly is shaped to receive the cartridge (and/or syringe and needle assembly) therewithin, with the cartridge (and/or syringe and needle assembly) being user-insertable.

9. A medicament injector according to claim 1, wherein the pod or cartridge assembly is resiliently biased to extend about a needle in a locked protected needle pre-injection mode and wherein the medicament is biased outwards from the needle when the pod or cartridge assembly is inserted into the applicator and the pod or cartridge assembly is moved from the locked protected needle pre-injection mode to a partially retracted mode which automatically moves the needle to a deployed position.

10. A medicament injector according to claim 8, wherein the medicament injector includes:
a first coupling mechanism via which the pod or cartridge assembly and the applicator couple together, the first coupling mechanism inhibiting axial movement of the pod or cartridge assembly outwardly of the applicator in a first instance and enabling retraction of a forward end portion of the pod or cartridge assembly relative to the applicator, which decouples the first coupling mechanism and initiates an automatic injection process;
a second coupling mechanism that inhibits axial movement of the cartridge (and/or syringe and needle assembly) relative to the pod or cartridge assembly and decouples upon initiation of the automatic injection process, to enable the needle assembly to move outwards of the pod or cartridge assembly to a deployed position via which the medicament is outputted therefrom; and
a third coupling mechanism which promotes positioning of the needle assembly in the deployed position until the medicament has been outputted from the cartridge (and/or syringe and needle assembly), with removable of inward pressure on the forward end portion of the pod or cartridge assembly thereafter enabling the pod or cartridge assembly to be automatically ejected from the applicator.

11. A medicament injector according to claim 1, including a safeguard mechanism coupled to the pod or cartridge assembly, the safeguard mechanism configured to enable axial movement of the pod or cartridge assembly relative to the applicator during an injection mode and upon the pod or cartridge assembly being removed from the applicator thereafter, being configured to inhibit reinsertion of the pod or cartridge assembly into the applicator once more.

12. A medicament injector comprising:
an applicator;
an interchangeable pod or cartridge assembly including or being shaped to receive a medicament-containing cartridge (and/or syringe and needle assembly); and
a barrier member coupled to the pod or cartridge assembly, wherein one or more of i) the barrier member is configured to inhibit access to the medicament until the pod or cartridge assembly is inserted within, coupled to and/or snap-fit connected to the applicator, and ii) the barrier member is configured such that removal thereof to enable access to the medicament requires the pod or cartridge assembly to be inserted within, coupled to and/or snap fit connected with the applicator.

13. A medicament injector according to claim 12, including one or more of:
wherein the medicament injector includes a locking mechanism that inhibits removal of the barrier member until the pod or cartridge assembly is inserted within, coupled to and/or snap-fit connected to the applicator; and
wherein the barrier member couples to the pod or cartridge assembly via a male member and a female member shaped to receive the male member, with insertion of the pod or cartridge assembly into the applicator causing the male member to dislodge from the female member at least in part.

14. A medicament injector according to claim 12, wherein the pod or cartridge assembly being received by and/or coupling to the applicator functions to automatically release the barrier member from the pod or cartridge assembly.

15. A medicament injector according to claim 12, including one or more of: wherein the barrier member is shaped to promote sterility of the pod or cartridge assembly and/or interior thereof; wherein the barrier member is shaped to extend about the needle in an airtight manner; and wherein the barrier member is shaped to inhibit inadvertent deployment of the needle when the pod or cartridge assembly is being inserted into the applicator.

16. A medicament injector according to claim 12, including one or more of: wherein the barrier member comprises a cap assembly; wherein the barrier member is telescoping; wherein the barrier member includes an outer cap and an inner cap telescopically coupled to the outer cap thereof; and wherein the medicament injector includes a needle via which the medicament is injectable, with the barrier member being configured to enable fluid communication between the needle and the medicament upon the pod or cartridge assembly being inserted within, coupled to and/or snap-fit connected to the applicator.

17. A medicament injector according to claim 12, including one or more of: wherein the needle assembly is configured to inhibit communication between the needle thereof and the medicament until the pod or cartridge assembly is inserted within, coupled to and/or snap fit connected with the applicator; wherein the needle assembly is telescoping; and wherein the needle assembly includes an outer hub and an inner hub, with the outer hub being shaped to receive the inner hub of the needle assembly upon the pod or cartridge assembly being inserted within, coupling to and/or snap fit connecting with the applicator.

18. A medicament injector according to claim 12, including one or more of:
wherein the needle assembly (and/or an inner hub thereof) is shaped to receive, couple with and/or snap fit connect with the cartridge so user-inserted into the pod or cartridge assembly;
wherein the needle assembly (and/or the inner hub thereof) includes a catch shaped to extend about and couple to the cartridge so user-inserted into the pod or cartridge assembly so as to inhibit removal of the cartridge thereafter; and
wherein the pod or cartridge assembly is configured to make an audible sound or click to indicate when the cartridge so user-inserted is properly locked into place.

19. A medicament injector comprising:
an interchangeable pod or cartridge assembly including a needle assembly, including a first telescoping mechanism and including or being shaped to receive a medicament-containing cartridge; and
an applicator shaped to receive the pod or cartridge assembly and actuate the first telescoping mechanism thereby to cause the needle assembly to pierce the cartridge, the applicator including a second telescoping mechanism actuation of which thereafter promotes outputting of the medicament from the needle.

20. A medicament injector comprising:
an applicator;
an interchangeable pod or cartridge assembly loadable into the applicator so as to couple thereto;
a medicament outputting means which automatically outputs a medicament from the pod or cartridge assembly upon subsequent retraction of the pod or cartridge relative to the applicator; and
an ejection means which automatically ejects the pod or cartridge assembly from the applicator after outputting of the medicament from the pod or cartridge assembly.
